(19) United States
(12) Patent Application Publication
(10) Pub. No.: US 2026/0157980 A1
(43) Pub. Date: Jun. 11, 2026

(54) METHODS FOR THE TREATMENT OF CANCER USING COENZYME Q10 IN COMBINATION WITH IMMUNE CHECKPOINT MODULATORS

(71) Applicant: BPGbio, Inc., Waltham, MA (US)

(72) Inventors: Maria-Dorothea Nastke, Holliston, MA (US); Shiva Kazerounian, Norwood, MA (US); Anne R. Diers, Wilmington, MA (US); Vivek K. Vishnudas, Chelmsford, MA (US); Stephane Gesta, Concord, MA (US); Rangaprasad Sarangarajan, Boylston, MA (US); Niven Rajin Narain, Cambridge, MA (US)

(21) Appl. No.: 19/271,132

(22) Filed: Jul. 16, 2025

Related U.S. Application Data
(63) Continuation of application No. 17/376,357, filed on Jul. 15, 2021, now abandoned.
Application No. 17/376,357 is a continuation of application No. 15/656,986, filed on Jul. 21, 2017, now abandoned.
(60) Provisional application No. 62/365,197, filed on Jul. 21, 2016.
(60) Provisional application No. 62/481,057, filed on Apr. 3, 2017.

(51) Int. Cl. A61K 31/122; A61K 9/00; A61K 9/06; A61K 9/08; A61K 31/704; A61K 39/395; A61K 47/02; A61K 47/10; A61K 47/24; A61K 47/26; C07K 16/28

(52) U.S. Cl. CPC A61K 31/122; A61K 9/0014; A61K 9/0019; A61K 9/06; A61K 9/08; A61K 31/704; A61K 39/39541; A61K 39/39558; A61K 39/39575; A61K 47/02; A61K 47/10; A61K 47/24; A61K 47/26; C07K 16/2818; C07K 16/2827; C07K 2317/21; C07K 2317/24; C07K 2317/76

(57) ABSTRACT

Presented herein are methods for the treatment of oncological disorders by the co-administration of Coenzyme Q10 and immune checkpoint modulators. The Coenzyme Q10 formulations may be at least one of intravenous, topical, or by inhalation. Co-administration of the Coenzyme Q10 formulations may be prior to, concurrent or substantially concurrent with, intermittent with or subsequent to the administration of the chemotherapy.

### RELATED APPLICATIONS

This application is a continuation of U.S. patent application Ser. No. 17/376,357, filed Jul. 15, 2021 which, in turn, is a continuation of U.S. patent application Ser. No. 15/656,986, filed Jul. 21, 2017 which, in turn, claims priority to U.S. Provisional Patent Application No. 62/365,197 filed on Jul. 21, 2016, and U.S. Provisional Patent Application No. 62/481,057 filed on Apr. 3, 2017, the contents of each of which are incorporated herein in their entirety.

### FIELD OF THE INVENTION

The invention generally relates to methods for the treatment of oncological disorders comprising administration of coenzyme Q10 (CoQ10) in combination with one or more modulators of an immune checkpoint molecule.

### BACKGROUND

Cancer is presently one of the leading causes of death in developed nations. A diagnosis of cancer traditionally involves serious health complications. Cancer can cause disfigurement, chronic or acute pain, lesions, organ failure, or even death. Commonly diagnosed cancers include pancreatic cancer, breast cancer, lung cancer, melanoma, lymphoma, carcinoma, sarcoma non-Hodgkin's lymphoma, leukemia, endometrial cancer, colon and rectal cancer, prostate cancer, and bladder cancer. Traditionally, many cancers (e.g., breast cancer, leukemia, lung cancer, or the like) are treated with surgery, chemotherapy, radiation, or combinations thereof. Chemotherapeutic agents used in the treatment of cancer are known to produce several serious and unpleasant side effects in patients. For example, some chemotherapeutic agents cause neuropathy, nephrotoxicity (e.g., hyperlipidemia, proteinuria, hypoproteinemia, combinations thereof, or the like), stomatitis, mucositisemesis, alopecia, anorexia, esophagitis amenorrhoea, decreased immunity, anaemia, high tone hearing loss, cardiotoxicity, fatigue, neuropathy, or combinations thereof. Thus a need exists for improved methods for the treatment of cancer.

### SUMMARY OF THE INVENTION

The present invention is based, at least in part, on the unexpected discovery that Coenzyme Q10 modulates expression of proteins involved in immune response in both T cells and cancer cells. Accordingly, the present invention provides methods for treating oncological disorders in a subject by administering CoQ10 and at least one immune checkpoint modulator of an immune check point molecule to the subject, such that the oncological disorder is treated. In certain embodiments, the immune checkpoint molecule is selected from the group consisting of CD27, CD28, CD40, CD122, OX40, GITR, ICOS, 4-1BB, ADORA2A, B7-H3, B7-H4, BTLA, CTLA-4, IDO, KIR, LAG-3, PD-1, PD-L1, PD-L2, TIM-3, and VISTA. In certain embodiments, the immune checkpoint molecule is selected from the group consisting of PD-1, PD-L1, PD-L2, CTLA-4, LAG-3, TIM-3 and VISTA. In certain embodiments, the immune checkpoint molecule is selected from the group consisting of PD-1, PD-L1 and CTLA-4. In certain embodiments, the immune checkpoint molecule is a stimulatory immune checkpoint molecule. In certain embodiments, the immune checkpoint modulator is an agonist of the stimulatory immune checkpoint molecule. In certain embodiments, the immune checkpoint molecule is an inhibitory immune checkpoint molecule. In certain embodiments, the immune checkpoint modulator is an antagonist of the inhibitory immune checkpoint molecule. In certain embodiments, the immune checkpoint modulator is selected from the group consisting of a small molecule, an inhibitory RNA, an antisense molecule, and an immune checkpoint binding protein. In certain embodiments, the immune checkpoint modulator is an immune checkpoint binding protein. In certain embodiments, the immune checkpoint binding protein is selected from the group consisting of an antibody, antibody Fab fragment, divalent antibody, antibody drug conjugate, scFv, fusion protein, bivalent antibody, and tetravalant antibody.

In certain embodiments, the immune checkpoint modulator is PD-1. In certain embodiments, the immune checkpoint modulator is selected from the group consisting of pembrolizumab, novolumab, pidilizumab, SHR-1210, MEDI0680R01, BBg-A317, TSR-042, REGN2810 and PF-06801591.

In one embodiment of the methods described herein, the immune checkpoint molecule is PD-L1. In certain embodiments, the immune checkpoint modulator is selected from the group consisting of durvalumab, atezolizumab, avelumab, MDX-1105, AMP-224 and LY3300054. In one embodiment, the immune checkpoint molecule is CTLA-4. In certain embodiments, the immune checkpoint modulator is selected from the group consisting of ipilimumab, tremelimumab, JMW-3B3 and AGEN1884. In one embodiment, the immune checkpoint molecule is LAG-3. In certain embodiments, the immune checkpoint modulator is selected from the group consisting of pembrolizumab, nivolumab, pidilizumab, SHR-1210, MEDI0680, PDR001, BGB-A317, TSR-042, REGN2810, and PF-06801591. In one embodiment, the immune checkpoint molecule is TIM-3. In certain embodiments, the immune checkpoint modulator is selected from the group consisting of TSR-022 and MGB453. In one embodiment, the immune checkpoint molecule is VISTA. In certain embodiments, the immune checkpoint modulator is selected from the group consisting of TSR-022 and MGB453.

In certain embodiments of the methods described herein, the Coenzyme Q10 is administered before administration of the immune checkpoint modulator. In certain embodiments, the Coenzyme Q10 is administered concurrently with the immune checkpoint modulator. In certain embodiments, the Coenzyme Q10 is administered after administration of the immune checkpoint modulator. In certain embodiments, a response of the oncological disorder to treatment is improved relative to a treatment with the at least one immune checkpoint modulator alone. In certain embodiments, the response in a population of patients is improved by at least 5%, at least 10%, at least 15%, at least 20%, at least 30%, at least 40%, at least 50%, at least 60%, at least 70%, at least 80% or more relative to treatment with the at least one immune checkpoint modulator alone. In certain embodiments, the response comprises any one or more of reduction in tumor burden, reduction in tumor size, inhibition of tumor growth, achieving stable oncological disorder in a subject with a progressive oncological disorder prior to treatment, increased time to progression of the oncological disorder, and increased time of survival. In certain embodiments, the Coenzyme Q10 and the immune checkpoint modulator act synergistically.

In certain embodiments of the methods described herein, the CoQ10 is administered topically. In certain embodiments, the CoQ10 is administered by injection or infusion. In certain embodiments, the CoQ10 is administered by intravenous administration. In certain embodiments, the CoQ10 is administered by continuous intravenous infusion. In certain embodiments, the CoQ10 is administered by continuous infusion over between 24 and 96 hours. In certain embodiments, the oncological disorder is selected from the group consisting of a carcinoma, sarcoma, lymphoma, melanoma, and leukemia. In certain embodiments, the oncological disorder is selected from the group consisting of pancreatic cancer, breast cancer, liver cancer, skin cancer, lung cancer, colon cancer, prostate cancer, thyroid cancer, bladder cancer, rectal cancer, endometrial cancer, kidney cancer, bone cancer, brain cancer, cervical cancer, stomach cancer, mouth and oral cancers, neuroblastoma, testicular cancer, uterine cancer, and vulvar cancer.

In certain embodiments, the skin cancer is selected from the group consisting of melanoma, squamous cell carcinoma, basal cell carcinoma, and cutaneous T-cell lymphoma (CTCL). In certain embodiments, the subject is human.

### BRIEF DESCRIPTION OF THE DRAWINGS

FIG. 1 shows a schematic of analysis to determine changes in T cell surface proteins expressed on cell components in buffy coat samples from cancer patients being administered Coenzyme Q10 for the treatment of solid tumors.

FIG. 2 shows differential expression of T cell surface proteins in response to Coenzyme Q10 treatment in buffy coat samples derived from patients afflicted with solid tumors. CD8B and CD247 were significantly downregulated, and CFL1 and S100A8 were significantly upregulated in response to Coenzyme Q10 treatment.

FIGS. 3A-3G show mRNA expression levels of PD-1, PD-L1 and PD-L2 in breast (MDA-MB231) (FIG. 3A), prostate (LnCAP) (FIG. 3B), ovarian (SKOV-3) (FIG. 3C), colon (HT29) (FIG. 3D), lung (A549) (FIG. 3E), liver (Huh-7) (FIG. 3F), or pancreatic (MIA PaCa-2) (FIG. 3G) cancer cells treated with Coenzyme Q10. There was a significant increase in PD-L1 mRNA expression in colon cancer cells treated with 50 μM Coenzyme Q10 relative to the untreated cells (*p<0.05; n=3). There were no significant differences among the other treatment groups.

FIGS. 4A and 4B show the results of flow cytometry analysis to determine the percentage of breast cancer cells (MDA-MB231) having PD-L1 protein on their surface. Coenzyme Q10 did not significantly change the percentage of breast cancer cells having PD-L1 protein on their surface 72 hours after treatment, as determined by unpaired t-test (n=10-12).

FIG. 5 shows the mean fluorescent intensity of PD-L1 protein on the surface of breast cancer cells (MDA-MB231) treated with Coenzyme Q10. Coenzyme Q10 treatment significantly increased the amount of PD-L1 protein on the surface of breast cancer cells 72 hours after treatment, as determined by unpaired t-test (n=10-12).

FIG. 6 shows PD-L1 protein expression on the surface of breast cancer cells (MDA-MB231) treated with Coenzyme Q10. Coenzyme Q10 treatment significantly increased the amount of PD-L1 protein on the surface of breast cancer cells 3 hours after treatment, as determined by unpaired t-test.

FIG. 7 shows PD-L1 protein expression on the surface of breast cancer cells (MDA-MB231) treated with 100 μM Coenzyme Q10 and 1 ng/mL doxorubicin. Co-treatment with Coenzyme Q10 and doxorubicin did not alter the amount of PD-L1 protein on the surface of breast cancer cells 72 hours after treatment.

FIG. 8 shows the results of flow cytometry analysis of breast cancer cells (MDA-MB231) treated with Coenzyme Q10 to determine the effect of Coenzyme Q10 on breast cancer cell populations. Coenzyme Q10 treatment did not change the size of the cell population.

FIG. 9 shows the results of flow cytometry analysis to determine the percentage of pancreatic cancer cells (MIA PaCa-2) having PD-L1 protein on their surface. Coenzyme Q10 significantly increased the percentage of pancreatic cancer cells having PD-L1 protein on their surface 72 hours after treatment, as determined by unpaired t-test (n=11-12).

FIG. 10 shows PD-L1 protein expression on the surface of pancreatic cancer cells (MIA PaCa-2) treated with Coenzyme Q10. Coenzyme Q10 treatment significantly increased the amount of PD-L1 protein on the surface of pancreatic cancer cells 72 hours after treatment, as determined by unpaired t-test.

FIG. 11 shows the results of flow cytometry analysis to determine the percentage of ovarian cancer cells (SKOV-3) having PD-L1 protein on their surface. Coenzyme Q10 did not significantly change the percentage of ovarian cancer cells having PD-L1 protein on their surface 72 hours after treatment (n=5-6).

FIG. 12 shows PD-L1 protein expression on the surface of ovarian cancer cells (SKOV-3) treated with Coenzyme Q10. Coenzyme Q10 caused a small but significant increase in the amount of PD-L1 protein on the surface of ovarian cancer cells 72 hours after treatment (n=5-6).

FIG. 13 shows the results of flow cytometry analysis to determine the percentage of lung cancer cells (A549) having PD-L1 protein on their surface. Coenzyme Q10 did not significantly change the percentage of lung cancer cells having PD-L1 protein on their surface 72 hours after treatment.

FIG. 14 shows PD-L1 protein expression on the surface of lung cancer cells (A549) treated with Coenzyme Q10. Coenzyme Q10 did not significantly alter the amount of PD-L1 protein on the surface of lung cancer cells 72 hours after treatment.

FIG. 15 shows a schematic representation of an ex vivo peripheral blood mononuclear cell (PBMC) model used to investigate the effect of Coenzyme Q10 on human immune cells. PBMCs isolated from healthy human donor leukopaks were isolated and cryopreserved. To study the effect of Coenzyme Q10, cells were thawed, rested overnight and treated with or without phytohemagglutinin (PHA). Various concentrations of Coenzyme Q10 (0, 12.5, 50, 200, 400 or 800 μM) were added to the cells at the same time. 24 hours to 72 hours post-treatment, frequency and viability of immune cell subpopulations was evaluated, as well as proliferative potential, cytokine secretion, and inhibitory immune checkpoint receptor surface expression.

FIGS. 16A-16E show the frequency of different human immune cell populations within PHA-stimulated or unstimulated PBMCs concurrently treated with Coenzyme Q10 (0, 12.5, 50, 200, 400 or 800 μM) evaluated by flow cytometry. 24 hours post treatment, PBMCs were analyzed for surface markers CD3/CD8, CD3/CD4, CD3/CD56, or CD19/CD14. Frequency of immune cell subtypes were graphed by percentage of cells gated for (A) T cells (CD3/CD8/CD4), (B) NKT cells, (C) NK cells, (D) B cells, and (E) monocytes. Data depicted are representative of 5 healthy donors tested.

FIGS. 17A-17E show the viability of human immune cell subpopulations within PHA-stimulated or unstimulated PBMCs concurrently treated with Coenzyme Q10. (A) Total, cytotoxic and helper T cell viability after treatment with increasing Coenzyme Q10 concentrations shows that T cell viability increases in response to Coenzyme Q10. Cells were treated with 0, 12.5, 50, 200, 400 or 800 μM Coenzyme Q10 for 24 hours. Cell populations and viability was determined by flow cytometry using combinational staining of surface markers. (B) CD3/CD8 cells, (C) CD3/CD4 cells, (D) CD3/CD56 cells, (E) CD19/CD14 cells and viability stains Annexin V/7 A AD. Data depicted are representative of 5 healthy donors tested.

FIGS. 18A and 18B show proliferation of human T cells assessed by flow cytometry using Click-iT EdU technology. PBMCs were incubated with our without PHA for 72 hours while concurrently treated with Coenzyme Q10 (200 μM). 10 μM of EdU was added for the final 18 hours and stained with Invitrogen Alexa Fluor 488 piclyl azide according to manufacturer's protocol. Cells were then stained with surface marker antibodies for CD3/CD8, or CD3/CD4 to identify cytotoxic T cells or helper T cells, respectively. Cells were then analyzyed by flow cytometry applying gating strategy as shown. (A) Histogram plots demonstrate clear separation of cells in S phase (DNA synthesis, including EdU incorporation) and cells in either G2/M or G0/G1. (B) Graphic display of T cell proliferation values acquired in (A). Data are representative of 2 donors tested.

FIG. 19 shows levels of the cytokines IL-2, interferon-7 (IFN-7) and IL-10 in supernatants of PHA-stimulated and rested human PBMCs concurrently treated with various concentrations of Coenzyme Q10. Cytokines were measured according to the manufacturer's protocol for R&D Quantikine ELISA kits (R&D Systems, Inc., Minneapolis, MN) specific to each cytokine. Shown are data of 3 donors tested.

FIGS. 20A and 20B show inhibitory receptor surface expression on human T cells within PBMCs treated with Coenzyme Q10 for 24 hours. Expression of immune checkpoint receptors were measured by staining cells with phenotypic markers for CD3/CD8, or CD3/CD4 in combination with antibodies against PD-1 or CTLA-4. Live cells were identified as 7 AAD negative lymphocytes followed by T cell phenotype characterization of total CD3+ T cells, cytotoxic T cells, or helper T cells, as indicated below plot. PD-1(A) or CTLA-4 (B) cell surface expression was measured as mean fluorescence intensity on live T cells. Data are representative of 3 donors tested.

FIGS. 21A and 21B show the viability of CD3 positive murine T cells within PHA-stimulated or unstimulated Balb/c PBMCs. Cells were concurrently treated with Coenzyme Q10 (0, 12.5, 50, 200, 400 or 800 μM) for 24 hours and analyzed by flow cytometry using surface marker antibody for αCD3 and viability stains Annexin V/7AAD. (A) CD3 positive and CD3 negative cell populations were identified within total cell population excluding debris and viability was determined by plotting Annexin V-FITC vs. 7AAD. (B) Graphed values as determined in (A). Data are representative of two experiments using two different pools of Balb/c PBMCs and one experiment using C57Bl/6 PBMCs.

FIG. 22 shows the frequency of PD-1 negative (PD-1⁻) and PD-1 high expressing (PD-1^{hi}) cells within PHA-stimulated or unstimulated Balb/c murine PBMCs. Cells were concurrently treated with Coenzyme Q10 (0, 12.5, 50, 200, 400 or 800 PM) for 24 hours and evaluated by flow cytometry using surface marker antibody for αCD3 and viability stains Annexin V/7AAD. Viable cells were identified by plotting Annexin V vs. 7 AAD, and gated viable cells were subjected to CD3 vs. PD-1 staining. Data are representative of two experiments using two different pools of Balb/c PBMCs and one experiment using C57Bl/6 PBMCs. Types of cells shown from left to right are unstimulated CD3⁻ PD-1⁻; unstimulated CD3⁺/PD-1⁻; unstimulated CD3⁺PD-1^{hi}; unstimulated CD3-PD-1^{hi}; stimulated CD3⁻ PD-1⁻; stimulated CD3⁺/PD-1⁻; stimulated CD3⁺PD-1^{hi}; and unstimulated CD3−PD-1^{hi}.

FIG. 23 shows PD-1 surface expression on CD3 positive murine T cells within PHA-stimulated or unstimulated Balb/c PBMCs. Cells were treated with Coenzyme Q10 (0, 12.5, 50, 200, 400 or 800 μM) for 24 hours and PD-1 expression was determined by gating live CD3 positive T cells. Mean fluorescence intensity values were evaluated in histogram plots for PD-1. Data are representative of two experiments using two different pools of Balb/c PBMCs and one experiment using C57Bl/6 PBMCs.

FIGS. 24A-24F show the sensitivity of mouse syngeneic tumor cell lines to Coenzyme Q10. Six mouse syngeneic tumor cell lines from different tissue types were exposed to increasing conentrations of Coenzyme Q10 (0-25 mM) at 37° C. for 72 hours. Cell viability was measured using CellTiter-Fluor kit (Promega, Madison, WI). Graphs and IC₅₀ values were calculated using GraphPad Prism using data for at least three independent experiments. Mouse syngeneic tumor cell lines evaluated were Lewis lung carcinoma (LL2) (A), hepatoma (Hepa1-6) (B), skin melanoma (B16F10) (C), colon cancer (CT26) (D), mammary gland adenocarcinoma (EMT6/P) (E), and renal adenocarcinoma (Renca) (F).

FIGS. 25A-25C show the effect of Coenzyme Q10 on the level of PD-L1 protein on the cell surface of mouse tumor cell lines. Mouse syngeneic tumor cell lines from different tissue types were cultured with or without INFγ in the presence or absence of their corresponding IC50 amount of Coenzyme Q10 at 37° C. for 24 hours. (A) Tumor cell lines with (+INFg) or without (−INFg) INFγ. (B) Tumor cell lines with (+CoQ10) or without Coenzyme Q10. (C) Tumor cell lines with INFγ and Coenzyme Q10 (INFg+CoQ10) or INFγ alone (INFg).

FIGS. 26A and 26B show C57BL/6 mice implanted with murine Pan02 pancreatic cancer cells and treated with different doses of Coenzyme Q10. C57Bl/6 female mice were inoculated with 3×10⁷ murine Pan02 pancreatic cancer cells. When tumors reached a mean volume of 100 mm³, animals were randomized into four groups and treated with vehicle control or Coenzyme Q10 (25, 50 or 100 mg/kg) twice daily for 21 days. Tumor volume was measured twice per week. (A) Overview of study design. (B) The 25, 50 and 100 mg/kg doses of Coenzyme Q10 decreased tumor volume by 7%, 19% and 26% respectively by Day 21.

FIG. 27 shows the body weight of C57BL/6 mice implanted with murine Pan02 pancreatic cancer cells and treated with Coenzyme Q10. Tumors with mean volume of 100 mm³ were treated twice per day with vehicle control or Coenzyme Q10 at 25, 50 or 100 mg/kg administered intraperitoneally for 21 days. Body weight was measured every two days for the first 5 days, and then twice per week. Coenzyme Q10 had no significant effect on the body weight of the animals.

FIGS. 28A and 28B show tumor samples from mice treated with different doses of Coenzyme Q10 analyzed for the presence of tumor associated macrophages (TAMs). TAMs are found in close proximity to or within tumors and support tumor growth. C57Bl/6 female mice were inoculated with murine Pan02 pancreatic cancer cells. When tumors reached a mean volume of 100 mm³, animals were randomized into four groups and treated with vehicle control or Coenzyme Q10 (25, 50 or 100 mg/kg) twice daily for 21 days. At the end of the study, tumors were removed and subjected to immunohistochemistry (IHC) analysis for TAMs using the F4/80 marker. All slides were subjected to a pathological scoring. Scores were relative to a control slide (from the control group) which demonstrated the best level of intensity. Coenzyme Q10 decreased TAMs in a dose dependent manner. (A) IHC analysis of tumor tissue. (B) Percentage of mice with TAM levels lower than control or similar to control.

FIGS. 29A and 29B show tumor samples from mice with murine Pan02 pancreatic tumors treated with different doses of Coenzyme Q10 and analyzed for the presence of tumor infiltrating lymphocytes (TILs). C57Bl/6 female mice were inoculated with 3×10⁷ Pan02 cells. When tumors reached a mean volume of 100 mm3, animals were randomized into four groups and treated with vehicle control or Coenzyme Q10 (25, 50 or 100 mg/kg) twice daily for 21 days. At the end of the study, tumors were removed and subjected to IHC analysis for Tumor Infiltrating Lymphocytes (TILs) with CD8 staining. All slides were subjected to a pathological scoring. Scores were relative to a control slide (from control group) which demonstrated the best level of intensity. Coenzyme Q10 increased TILs in a dose-dependent manner. (A) IHC analysis of tumor tissue. Arrows indicate the presence of TILs. (B) Percentage of mice with TIL levels higher than control, lower than control, or similar to control.

FIG. 30 shows differential expression of proteins within buffy coat samples from cancer patients treated with Coenzyme Q10 based on assignment of tumor slopes to identify shrinking and growing tumors.

### DETAILED DESCRIPTION

The immune checkpoint modulator (e.g., inhibitor) therapies approved to date have demonstrated clinical responses in multiple tumor types and are continuously being evaluated for broader utility. However, in spite of the remarkable responses observed in multiple cancers in response to targeting a single checkpoint on immune cells, the durability of response has been observed only in a fraction of patients. Efforts are currently focused on targeting multiple checkpoints using combination therapy based on the bifurcation in T cell pathways targeted by various immunotherapies to improve durable anti-tumor responses in the clinical setting. Clinical trials of combination therapies are ongoing with long term patient outcomes yet to be determined. See Sharma et al., cited above.

T cell mediated immune responses involve a sequence of events that require clonal selection of antigen specific cells, their activation and proliferation, transport to the site of the antigen and elicitation of immune response. See Mockler et al., 2014, Frontiers in Oncol 4:1; and Pearce et al., 2013, Science 342(6155):1242454, each of which is incorporated by reference herein. Upon receiving T cell receptor and co-stimulatory signals, T cells develop in growth, expansion and differentiation into cytotoxic, regulatory, or helper T cells. Depending on their stage of activation, T cells display distinct metabolic profiles. See Mockler et al., 2014, Front. Oncol. 4: 107, which is incorporated by reference herein in its entirety. Naïve T cells are metabolically quiescent adopting a basal level of nutrient uptake and rely on oxidative phosphorylation as a primary source for ATP production. In contrast, activated T cells (effector T cells) adopt an anabolic metabolic profile to guarantee increased energy supplies needed for cell growth, proliferation, differentiation, and effector functions. Effector T cells preferentially use glycolysis over oxidative phosphorylation for ATP production, therefore consuming high amounts of glucose. Contrary to naïve T cells and effector T cells, the long lifespan of memory T cells poses a different metabolic demand. Transition to the memory stage is characterized by a quiescent metabolism with an increased reliance on fatty acid oxidation to fuel oxidative phosphorylation. In summary, each stage of T cell development requires metabolic support via production of energy and generation of biosynthetic precursors. Thus it is critical that T cells undergo appropriate activation and differentiation to maintain homeostasis.

T cells in tumors, so-called tumor infiltrating lymphocytes (TILs) have been shown to be key denominators for overall survival in solid cancer bearing patients. The tumor microenvironment is hostile to T cell function, e.g. due to expression of enzymes that deplete the amino acids tryptophan and arginine and the presence of innate cells or regulatory T cells which both have suppressive activity. Moreover, cancer cells are characterized by an altered metabolism, glycolysis, in which glucose is metabolized to lactate which is secreted to the microenvironment rather than further metabolized in the mitochondria. This altered metabolism is governed by activated oncogenes and/or hypoxia. Lactate negatively impacts the function of immune cells and it is detrimental to T cell function, cytokine production and cytokine capacity. See Droge et al., 1987, Cell Immunol 108(2):405-16; and Fischer et al., 2007, Blood 109(9):3812-9.

The unique bioenergetics challenge within the tumor microenvironment can range from extreme hypoxic regions to areas of aerobic glycolysis rendering the microenvironment nutrient deficient. See Mockler et al., cited above. Each of these conditions can have a profound effect on T cell function and thus impair anti-tumor immune responses. Hypoxia associated changes in tumor microenvironment can lead to a decrease in T cell proliferation, downregulate mitochondrial oxygen consumption, and impact differentiation leading to a perpetual low level of inflammation. Furthermore, nutrient deprivation can limit the availability of substrates such as glucose that is essential for effector T cell survival and proliferation.

A central part of the T cell activation involves significant alterations in cellular metabolism including a marked increase in glucose metabolism. Although glycolysis represents a rapid source of ATP generation along with NADPH via the pentose shunt, it is not sufficient to generate the full complement of molecules essential for proliferation. However, increased mitochondrial oxygen consumption along with generation of ROS is essential for T cell activation and differentiation. Furthermore, mitochondrial ATP released in the extracellular space enables purinergic signaling mechanisms that regulate T cell activation in the immune—APC synapse. Normal mitochondrial function represents a central role in harnessing of immune response since primary mitochondrial dysfunction is associated with immune dysfunction and increased incidence of infections. See Ledderose et al., 2014, J Biol Chem 289:25936; and Sena et al., 2013, Immunity 38:2 25.

One of the hallmarks of cancer is evasion of the immune system, so cancer immunotherapy must take a different approach by augmenting the beneficial anti-tumor responses of effector T cells initially, leading to memory T cell generation and by attenuating the responses of regulatory T cells. Increasing activated tumor specific effector T cell numbers is perhaps the most beneficial approach to elevate anti-tumor immunity.

Coenzyme Q10 has been described previously as an anti-cancer thereapeutic agent (see, e.g, PCT/US2005/001581, the entire contents of which are incorporated herein by reference), and is being evaluated in humans as mono-therapy or in combination with standard of care chemotherapy agents for treatment of solid tumors. The results presented herein demonstrate that Coenzyme Q10 effects significant changes in the levels of four T cell surface proteins (CD8B, CD247, CFL1, and S100A8) in cancer patients administered Coenzyme Q10. For example, expression of CD8B and CD247 was downregulated by Coenzyme Q10 treatment, and expression of CFL1 and S100A8 was upregulated by Coenzyme Q10 treatment in these patients (see Example 1). These results indicate that Coenzyme Q10 plays a role in modulating the immune response in cancer patients. In addition, the results presented herein demonstrate that Coenzyme Q10 treatment increased cell surface levels of PD-L1 in human cancer cells that express moderate to high levels of PD-L1 before treatment (see Example 2). Thus Coenzyme Q10 was demonstrated to modulate expression of proteins involved in immune response in both T cells and cancer cells. Furthermore, Coenzyme Q10 dose-dependently increased the frequency and viability of human CD3+ T cells, and increased proliferation of PHA-activated cytotoxic T cells (see Example 6) and increased the level of TILs and decreases the level of TAMs in a syngeneic pancreatic cancer model (see Example 10).

While not wishing to be bound by theory, Coenzyme Q10 may modulate an immune response against a tumor through its effects on cancer cell metabolism. For example, Coenzyme Q10 has a unique mechanism of action in that it effectuates an anti-Warburg switch in cancer cell metabolism, i.e., switching cancer bioenergetics demands from glycolysis to mitochondrial oxidative phosphorylation. This phenomenon elicited by Coenzyme Q10 is typically associated with an increase in mitochondrial reactive oxygen species (ROS) generation and activation of apoptosis. Coenzyme Q10 rapidly accumulates in various intracellular compartments including the plasma membrane, cytoplasm and intracellular organelles, with a several fold higher concentration observed within the mitochondria. As discussed above, effector T cells display a high demand for glucose to support activation, proliferation and effector functions. There is evidence that effector T cells compete with tumor cells for available glucose in the tumor microenvironment, and this competition model of nutrient restriction limits the ability of effector T cells to produce effector cytokines such as IFN-7. See Chang et al., 2013, Cell 153(6):1239-51. Tumor-derived lactate is also able to suppress cytotoxic T cell function by directly blocking lactate export by T cells resulting in their inability to maintain glycolysis. See Fischer et al., 2007, Blood 109(9):3812-9. Coenzyme Q10 induced apoptosis of cancer cells will result in higher glucose levels in the tumor thus providing a higher energy supply for effector T cells to thereby benefit cell growth, proliferation, differentiation, and effector functions. A higher activation state of effector T cells may result in increased levels of cytotoxic effector molecules (e.g. perforin, granzymes, Fas ligand) and macrophage activating effector molecules (e.g. IFN-7, GM-CSF, TNF-α, IL-2) which supports and attracts other immune cells (e.g. NK cells) to the site of response against tumor cells.

Based upon the results presented herein, Coenzyme Q10 and immune checkpoint modulator therapies are expected to work particularly effectively in concert for the treatment of cancers. For example, combination of Coenzyme Q10 with immune checkpoint inhibitors has the potential to synergize the activity of these agents in augmenting T cell mediated anti-tumor responses, thereby improving overall durability in patient outcomes. Accordingly, the present invention provides methods for treating oncological disorders in a subject in need thereof by administering to the subject CoQ10 and at least one modulator of an immune check point molecule.

### I. Definitions

In accordance with the present disclosure and as used herein, the following terms are defined with the following meanings, unless explicitly stated otherwise.

As used herein, an “immune checkpoint” or “immune checkpoint molecule” is a molecule in the immune system that modulates a signal. An immune checkpoint molecule can be a stimulatory checkpoint molecule, i.e., turn up a signal, or inhibitory checkpoint molecule, i.e., turn down a signal. A “stimulatory checkpoint molecule” as used herein is a molecule in the immune system that turns up a signal or is co-stimulatory. An “inhibitory checkpoint molecule”, as used herein is a molecule in the immune system that turns down a signal or is co-inhibitory.

As used herein, an “immune checkpoint modulator” is an agent capable of altering the activity of an immune checkpoint in a subject. In certain embodiments, an immune checkpoint modulator alters the function of one or more immune checkpoint molecules including CD27, CD28, CD40, CD122, OX40, GITR, ICOS, 4-1BB, ADORA2A, B7-H3, B7-H4, BTLA, CTLA-4, IDO, KIR, LAG-3, PD-1, PD-L1, PD-L2, TIM-3, and VISTA. The immune checkpoint modulator may be an agonist or an antagonist of the immune checkpoint. In some embodiments, the immune checkpoint modulator is an immune checkpoint binding protein (e.g., an antibody, antibody Fab fragment, divalent antibody, antibody drug conjugate, scFv, fusion protein, bivalent antibody, or tetravalent antibody). In other embodiments, the immune checkpoint modulator is a small molecule. In a particular embodiment, the immune checkpoint modulator is an anti-PD1, anti-PD-L1, or anti-CTLA-4 antibody. In a further particular embodiment, the immune checkpoint modulator is an anti-PD-1 antibody or anti-PD-L1 antibody.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, “continuous infusion” is understood as administration of a therapeutic agent continuously for a period of at least 24 hours. Continuous infusion is typically accomplished by the use of a pump, optionally an implantable pump. A continuous infusion may be administered within the context of a treatment cycle. For example, a dose of a therapeutic agent can be administered by continuous infusion over a 24 hour period once per week each week. Treatment with continuous infusion does not require infusion of the therapeutic agent to the subject for the entire treatment period.

It is understood that continuous infusion can include short interruptions of administration, for example, to change the reservoir of coenzyme Q10 being administered. Continuous administration is typically facilitated by the use of a pump. Continuous infusion is carried out without including any significant interruptions of dosing by design. As used herein, interruptions to assess vital signs and/or perform laboratory assessments to ensure the safety of the patients and that no unacceptable adverse event have occurred are not considered to be significant interruptions. Interruptions resulting from equipment failure, e.g., pump failure, are not interruptions by design.

As used herein, “oncological disorder”, “cancer” or “tumor” refers to all types of cancer or neoplasm or malignant tumors found in humans, including, but not limited to: leukemias, lymphomas, melanomas, carcinomas and sarcomas. As used herein, the terms or language “oncological disorder”, “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also cancer stem cells, as well as cancer progenitor cells or any cell derived from a cancer cell ancestor. This includes metastasized cancer cells, and in vitro cultures and cell lines derived from cancer cells.

A “solid tumor” is a tumor that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. The tumor does not need to have measurable dimensions.

When referring to a type of cancer that normally manifests as a solid tumor, a “clinically detectable” tumor is one that is detectable on the basis of tumor mass, e.g., by procedures such as CAT scan, MR imaging, X-ray, ultrasound or palpation, and/or which is detectable because of the expression of one or more cancer-specific antigens in a sample obtainable from a patient.

As used herein, a “detectable tumor” is a tumor that can be confirmed to be present in a subject, for example, using imaging methods (e.g., x-ray, CT scan, magnetic resonance imaging either with or without contrast agents, ultrasound), palpation or other physical examination methods, and/or direct observation by surgical methods or biopsy, typically coupled with histological analysis, in the case of a solid tumors; or by analysis of blood samples, e.g., complete blood count or histological analysis in the case of non-solid tumors, e.g., leukemias. In certain embodiments, a tumor can be detected based on the presence or certain markers. It is understood that diagnosis and detection of a tumor may involve multiple tests and diagnostic methods.

The term “sarcoma” generally refers to a tumor which is made up of a substance like the embryonic connective tissue and is generally composed of closely packed cells embedded in a fibrillar or homogeneous substance. Examples of sarcomas which can be treated with the methods of the invention include, for example, a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated with the methods of the invention include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Carcinomas which can be treated with the methods of the invention, as described herein, include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, merkel cell carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “leukemia” refers to a type of cancer of the blood or bone marrow characterized by an abnormal increase of immature white blood cells called “blasts”. Leukemia is a broad term covering a spectrum of diseases. In turn, it is part of the even broader group of diseases affecting the blood, bone marrow, and lymphoid system, which are all known as hematological neoplasms. Leukemias can be divided into four major classifications, acute lymphocytic (or lymphoblastic) leukemia (ALL), acute myelogenous (or myeloid or non-lymphatic) leukemia (AML), chronic lymphocytic leukemia (CLL), and chronic myelogenous leukemia (CML). Further types of leukemia include Hairy cell leukemia (HCL), T-cell prolymphocytic leukemia (T-PLL), large granular lymphocytic leukemia, and adult T-cell leukemia.

The term “lymphoma” refers to a group of blood cell tumors that develop from lymphatic cells. The two main categories of lymphomas are Hodgkin lymphomas (HL) and non-Hodgkin lymphomas (NHL) Lymphomas include any neoplasms of the lymphatic tissues. The main classes are cancers of the lymphocytes, a type of white blood cell that belongs to both the lymph and the blood and pervades both.

Specific criteria for the staging of cancer are dependent on the specific cancer type based on tumor size, histological characteristics, tumor markers, and other criteria known by those of skill in the art. Generally, cancer stages can be described as follows:
Stage 0 Carcinoma in situ
Stage I, Stage II, and Stage III Higher numbers indicate more extensive disease: Larger tumor size and/or spread of the cancer beyond the organ in which it first developed to nearby lymph nodes and/or tissues or organs adjacent to the location of the primary tumor
Stage IV The cancer has spread to distant tissues or organs

As used herein, the terms “treat,” “treating” or “treatment” refer, preferably, to an action to obtain a beneficial or desired clinical result including, but not limited to, alleviation or amelioration of one or more signs or symptoms of a disease or condition (e.g., regression, partial or complete), diminishing the extent of disease, stability (i.e., not worsening, achieving stable disease) of the state of disease, amelioration or palliation of the disease state, diminishing rate of progression or increasing time to progression, and remission (whether partial or total). “Treatment” of a cancer can also mean prolonging survival as compared to expected survival in the absence of treatment. Treatment need not be curative. In certain embodiments, treatment includes one or more of a decrease in pain or an increase in the quality of life (QOL) as judged by a qualified individual, e.g., a treating physician, e.g., using accepted assessment tools of pain and QOL. In certain embodiments, treatment does not include one or more of a decrease in pain or an increase in the quality of life (QOL) as judged by a qualified individual, e.g., a treating physician, e.g., using accepted assessment tools of pain and QOL.

As used herein, “treatment” refers to a symptom or sign which approaches a normalized value (for example a value obtained in a healthy patient or individual), e.g., is less than 50% different from a normalized value, in embodiments less than about 25% different from a normalized value, in other embodiments is less than 10% different from a normalized value, and in yet other embodiments the presence of a symptom is not significantly different from a normalized value as determined using routine statistical tests. As used herein, treatment can include reduction of tumor burden, inhibition of tumor growth, including inducing stable disease in a subject with progressive disease prior to treatment, increasing time to progression, or increasing survival time. Increases can be determined relative to an appropriate control or expected outcomes. As used herein, treatment can include increasing survival of a subject, with or without a decrease in tumor burden, as compared to appropriate controls. Treatment need not be curative.

RECIST criteria are clinically accepted assessment criteria used to provide a standard approach to solid tumor measurement and provide definitions for objective assessment of change in tumor size for use in clinical trials. Such criteria can also be used to monitor response of an individual undergoing treatment for a solid tumor. The RECIST 1.1 criteria are discussed in detail in Eisenhauer et al., New response evaluation criteria in solid tumors: Revised RECIST guideline (version 1.1). Eur. J. Cancer. 45:228-247, 2009, which is incorporated herein by reference. Response criteria for target lesions include:
Complete Response (CR): Disappearance of all target lesions. Any pathological lymph nodes (whether target or non-target) must have a reduction in short axis to <10 mm.
Partial Response (PR): At least a 30% decrease in the sum of diameters of target lesion, taking as a reference the baseline sum diameters.
Progressive Diseases (PD): At least a 20% increase in the sum of diameters of target lesions, taking as a reference the smallest sum on the study (this includes the baseline sum if that is the smallest on the study). In addition to the relative increase of 20%, the sum must also demonstrate an absolute increase of at least 5 mm. (Note: the appearance of one or more new lesions is also considered progression.)
Stable Disease (SD): Neither sufficient shrinkage to qualify for PR nor sufficient increase to qualify for PD, taking as a reference the smallest sum diameters while on study.

RECIST 1.1 criteria also consider non-target lesions which are defined as lesions that may be measureable, but need not be measured, and should only be assessed qualitatively at the desired time points. Response criteria for non-target lesions include:

Complete Response (CR): Disappearance of all non-target lesions and normalization of tumor marker levels. All lymph nodes must be non-pathological in size (<10 mm short axis).

Non-CR/Non-PD: Persistence of one or more non-target lesion(s) and/or maintenance of tumor marker level above the normal limits.

Progressive Disease (PD): Unequivocal progression (emphasis in original) of existing non-target lesions. The appearance of one or more new lesions is also considered progression. To achieve “unequivocal progression” on the basis of non-target disease, there must be an overall level of substantial worsening of non-target disease such that, even in the presence of SD or PR in target disease, the overall tumor burden has increased sufficiently to merit discontinuation of therapy. A modest “increase” in the size of one or more non-target lesions is usually not sufficient to qualify for unequivocal progression status. The designation of overall progression solely on the basis of change in non-target disease in the face of SD or PR in target disease will therefore be extremely rare.

Clinically acceptable criteria for response to treatment in acute leukemias are as follows:
Complete remission (CR): The patient must be free of all symptoms related to leukemia and have an absolute neutrophil count of ≥1.0×10⁹/L, platelet count ≥100×10⁹/L, and normal bone marrow with <5% blasts and no Auer rods.
Complete remission with incomplete blood count recovery (Cri): As per CE, but with residual thrombocytopenia (platelet count <100×10⁹/L) or residual neutropenia (absolute neutrophil count <1.0×10⁹/L).
Partial remission (PR): A ≥50% decrease in bone marrow blasts to 5 to 25% abnormal cells in the marrow; or CR with ≤5% blasts if Auer rods are present.
Treatment failure: Treatment has failed to achieve CR, Cri, or PR. Recurrence.
Relapse after confirmed CR: Reappearance of leukemic blasts in peripheral blood or ≥5% blasts in the bone marrow not attributable to any other cause (e.g., bone marrow regeneration after consolidated therapy) or appearance of new dysplastic changes.

As used herein, “co-administration” or “combination therapy” is understood as administration of two or more active agents using separate formulations or a single pharmaceutical formulation, or consecutive administration in any order such that, there is a time period while both (or all) active agents simultaneously exert their biological activities. It is contemplated herein that one active agent (e.g., CoQ10) can improve the activity of a second agent, for example, can sensitize target cells, e.g., cancer cells, to the activities of the second agent. Co-administration does not require that the agents are administered at the same time, at the same frequency, or by the same route of administration. As used herein, “co-administration” or “combination therapy” includes administration of a CoQ10 compound with one or more additional anti-cancer agents, e.g., immune checkpoint modulators. Examples of immune checkpoint modulators are provided herein.

A “subject who has failed a chemotherapeutic regimen” is a subject with cancer that does not respond, or ceases to respond to treatment with a chemotherapeutic regimen per RECIST 1.1 criteria (see, Eisenhauer et al., 2009 and as discussed above), i.e., does not achieve at least stable disease (i.e., stable disease, partial response, or complete response) in the target lesion; or does not achieve at least non-CR/non-PD (i.e., non-CR/non-PD or complete response) of non-target lesions, either during or after completion of the chemotherapeutic regimen, either alone or in conjunction with surgery and/or radiation therapy which, when possible, are often clinically indicated in conjunction with chemotherapy. A failed chemotherapeutic regime results in, e.g., tumor growth, increased tumor burden, and/or tumor metastasis. In some embodiments, failed chemotherapeutic regimen as used herein includes a treatment regimen that was terminated due to a dose limiting toxicity, e.g., a grade III or a grade IV toxicity that cannot be resolved to allow continuation or resumption of treatment with the chemotherapeutic agent or regimen that caused the toxicity. In some embodiments, a “failed chemotherapeutic regimen includes a treatment regimen that does not result in at least stable disease for all target and non-target lesions for an extended period, e.g., at least 1 month, at least 2 months, at least 3 months, at least 4 months, at least 5 months, at least 6 months, at least 12 months, at least 18 months, or any time period less than a clinically defined cure. In some embodiments, a failed chemotherapeutic regimen includes a treatment regimen that results in progressive disease of at least one target lesion during treatment with the chemotherapeutic agent, or results in progressive disease less than 2 weeks, less than 1 month, less than two months, less than 3 months, less than 4 months, less than 5 months, less than 6 months, less than 12 months, or less than 18 months after the conclusion of the treatment regimen, or less than any time period less than a clinically defined cure.

A failed chemotherapeutic regimen does not include a treatment regimen wherein the subject treated for a cancer achieves a clinically defined cure, e.g., 5 years of complete response after the end of the treatment regimen, and wherein the subject is subsequently diagnosed with a distinct cancer, e.g., more than 5 years, more than 6 years, more than 7 years, more than 8 years, more than 9 years, more than 10 years, more than 11 years, more than 12 years, more than 13 years, more than 14 years, or more than 15 years after the end of the treatment regimen. For example, a subject who suffered from a pediatric cancer may develop cancer later in life after being cured of the pediatric cancer. In such a subject, the chemotherapeutic regimen to treat the pediatric cancer is considered to have been successful.

A “refractory cancer” is a malignancy for which surgery is ineffective, which is either initially unresponsive to chemo- or radiation therapy, or which becomes unresponsive to chemo- or radiation therapy over time.

The terms “administer”, “administering” or “administration” include any method of delivery of a pharmaceutical composition or agent into a subject's system or to a particular region in or on a subject. In certain embodiments, the agent is delivered orally. In certain embodiments, the agent is administered parenterally. In certain embodiments, the agent is delivered by injection or infusion. In certain embodiments, the agent is delivered topically including transmucosally. In certain embodiments, the agent is delivered by inhalation. In certain embodiments of the invention, an agent is administered by parenteral delivery, including, intravenous, intramuscular, subcutaneous, intramedullary injections, as well as intrathecal, direct intraventricular, intraperitoneal, intranasal, or intraocular injections. In one embodiment, the compositions provided herein may be administered by injecting directly to a tumor. In some embodiments, the formulations of the invention may be administered by intravenous injection or intravenous infusion. In certain embodiments, the formulation of the invention can be administered by continuous infusion. In certain embodiments, administration is not oral. In certain embodiments, administration is systemic. In certain embodiments, administration is local. In some embodiments, one or more routes of administration may be combined, such as, for example, intravenous and intratumoral, or intravenous and peroral, or intravenous and oral, intravenous and topical, or intravenous and transdermal or transmucosal. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, continuous infusion, etc.

“Adverse events” or “AEs” are characterized by grade depending on the severity. Some AE (e.g., nausea, low blood counts, pain, reduced blood clotting) can be treated so that the specific chemotherapeutic regimen can be continued or resumed. Some adverse events (e.g., loss of cardiac, liver, or kidney function; nausea) may not be treatable, requiring termination of treatment with the drug. Determination of AE grade and appropriate interventions can be determined by those of skill in the art. Common Terminology Criteria for Adverse Events v4.0 (CTCAE) (Publish Date: May 28, 2009) provide a grading scale for adverse events as follows:
Grade 1 Mild; asymptomatic or mild symptoms; clinical or diagnostic observations only; intervention not indicated.
Grade 2 Moderate; minimal, local or noninvasive intervention indicated; limiting age-appropriate instrumental activities of daily life (ADL).
Grade 3 Severe or medically significant but not immediately life-threatening; hospitalization or prolongation of hospitalization indicated; disabling, limiting self care ADL.
Grade 4 Life-threatening consequences; urgent intervention indicated.
Grade 5 Death related to adverse event.

As used herein, the term “survival” refers to the continuation of life of a subject which has been treated for a disease or condition, e.g., cancer. The time of survival can be defined from an arbitrary point such as time of entry into a clinical trial, time from completion or failure or an earlier treatment regimen, time from diagnosis, etc.

As used herein, a “dispersion” refers to a system in which particles of colloidal size of any nature (e.g., solid, liquid or gas) are dispersed in a continuous phase of a different composition or state. In intravenous drug delivery the continuous phase is substantially water and the dispersed particles can be solid (a suspension) or an immiscible liquid (emulsion).

A “subject” to be treated by the method of the invention can mean either a human or non-human animal, preferably a mammal, more preferably a human. In certain embodiments, a subject has a detectable tumor prior to initiation of treatments using the methods of the invention. In certain embodiments, the subject has a detectable tumor at the time of initiation of the treatments using the methods of the invention.

As used herein, the term “safe and therapeutic effective amount” refers to the quantity of a component which is sufficient to yield a desired therapeutic response without undue adverse side effects (such as toxicity, irritation, or allergic response) commensurate with a reasonable benefit/risk ratio when used in the manner of this disclosure.

“Therapeutically effective amount” means the amount of a compound that, when administered to a patient for treating a disease, is sufficient to effect such treatment for the disease. When administered for preventing a disease, the amount is sufficient to avoid or delay onset of the disease. The “therapeutically effective amount” will vary depending on the compound, the disease and its severity and the age, weight, etc., of the patient to be treated. A therapeutically effective amount need not be curative. A therapeutically effective amount need not prevent a disease or condition from ever occurring. Instead a therapeutically effective amount is an amount that will at least delay or reduce the onset, severity, or progression of a disease or condition. Disease progression can be monitored, for example, by one or more of tumor burden, time to progression, survival time, or other clinical measurements used in the art.

The term “therapeutic effect” refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically-effective amount of a compound will depend on its therapeutic index, solubility, and the like.

“Preventing” or “prevention” refers to a reduction in risk of acquiring a disease or disorder (i.e., causing at least one of the clinical signs or symptoms of the disease not to develop in a patient that may be exposed to or predisposed to the disease but does not yet experience or display symptoms of the disease). Prevention does not require that the disease or condition never occur, or recur, in the subject.

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

In all occurrences in this application where there are a series of recited numerical values, it is to be understood that any of the recited numerical values may be the upper limit or lower limit of a numerical range. It is to be further understood that the invention encompasses all such numerical ranges, i.e., a range having a combination of an upper numerical limit and a lower numerical limit, wherein the numerical value for each of the upper limit and the lower limit can be any numerical value recited herein. Ranges provided herein are understood to include all values within the range. For example, 1-10 is understood to include all of the values 1, 2, 3, 4, 5, 6, 7, 8, 9, and 10, and fractional values as appropriate. Ranges expressed as “up to” a certain value, e.g., up to 5, is understood as all values, including the upper limit of the range, e.g., 0, 1, 2, 3, 4, and 5, and fractional values as appropriate. Up to or within a week is understood to include, 0.5, 1, 2, 3, 4, 5, 6, or 7 days. Similarly, ranges delimited by “at least” are understood to include the lower value provided and all higher numbers.

All percent formulations are w/w unless otherwise indicated.

As used herein, “about” is understood to include within three standard deviations of the mean or within standard ranges of tolerance in the specific art. In certain embodiments, about is understood a variation of no more than 0.5.

The articles “a” and “an” are used herein to refer to one or to more than one (i.e. to at least one) of the grammatical object of the article. By way of example, “an element” means one element or more than one element.

The term “including” is used herein to mean, and is used interchangeably with, the phrase “including but not limited to”.

The term “or” is used inclusively herein to mean, and is used interchangeably with, the term “and/or,” unless context clearly indicates otherwise.

The term “such as” is used herein to mean, and is used interchangeably, with the phrase “such as but not limited to”.

### II. Immunotherapy in Cancer

The ability of tumor cells to harness a range of complex, overlapping mechanisms to prevent the immune system from distinguishing self from non-self represents the fundamental mechanism of tumors to evade immunesurveillance. Mechanism(s) include disruption of antigen presentation, disruption of regulatory pathways controlling T cell activation or inhibition (immune checkpoint regulation), recruitment of cells that contribute to immune suppression (Tregs, MDSC) or release of factors that influence immune activity (IDO, PGE2). See Harris et al., 2013, J Immunotherapy Cancer 1:12; Chen et al., 2013, Immunity 39:1; Pardoll, et al., 2012, Nature Reviews: Cancer 12:252; and Sharma et al., 2015, Cell 161:205, each of which is incorporated by reference herein in its entirety. Recent years have seen an explosion of immune-oncology therapeutic modalities with approaches ranging from inhibitors of T cell checkpoint, T cell activating agents, and potential vaccines either approved for clinical use or under active investigation. A few of these, including anti-CTLA-4, anti-PD-1, and anti-PD-L1 immune checkpoint therapies, have demonstrated variable success and have been approved for clinical use. Although the checkpoint inhibitors are the most advanced in clinical development for treatment of various cancers, these represent a fraction of the potential targets and pathways that can be harnessed to improve anti-tumor responses. This is evidenced by the continuous emergence of new lists of potential molecules influencing checkpoint or inhibitory pathways along with co-stimulatory molecules that improve immune responses that are in various stages of pre-clinical and clinical development. Examples of new immune checkpoints that are being evaluated for cancer treatment include LAG-3 (Triebel et al., 1990, J. Exp. Med. 171: 1393-1405), TIM-3 (Sakuishi et al., 2010, J. Exp. Med. 207: 2187-2194) and VISTA (Wang et al., 2011, J. Exp. Med. 208: 577-592). Examples of co-stimulatory molecules that improve immune responses include ICOS (Fan et al., 2014, J. Exp. Med. 211: 715-725), OX40 (Curti et al., 2013, Cancer Res. 73: 7189-7198) and 4-1BB (Melero et al., 1997, Nat. Med. 3: 682-685).

Immune checkpoints of the invention may be stimulatory immune checkpoints (i.e. molecules that stimulate the immune response) or inhibitory immune checkpoints (i.e. molecules that inhibit immune response). In some embodiments, the immune checkpoint modulator is an antagonist of an inhibitory immune checkpoint. In some embodiments, the immune checkpoint modulator is an agonist of a stimulatory immune checkpoint. In some embodiments, the immune checkpoint modulator is an immune checkpoint binding protein (e.g., an antibody, antibody Fab fragment, divalent antibody, antibody drug conjugate, scFv, fusion protein, bivalent antibody, or tetravalent antibody). In certain embodiments, the immune checkpoint modulator is capable of binding to, or modulating the activity of more than one immune checkpoint. Examples of stimulatory and inhibitory immune checkpoints, and molecules that modulate these immune checkpoints that may be used in the methods of the invention, are provided below.

### Stimulatory Immune Checkpoint Molecules

CD27 supports antigen-specific expansion of naïve T cells and is vital for the generation of T cell memory (see, e.g., Hendriks et al. (2000) Nat. Immunol. 171 (5): 433-40). CD27 is also a memory marker of B cells (see, e.g., Agematsu et al. (2000) Histol. Histopathol. 15 (2): 573-6. CD27 activity is governed by the transient availability of its ligand, CD70, on lymphocytes and dendritic cells (see, e.g., Borst et al. (2005) Curr. Opin. Immunol. 17 (3): 275-81). Multiple immune checkpoint modulators specific for CD27 have been developed and may be used as disclosed herein. In some embodiments, the immune checkpoint modulator is an agent that modulates the activity and/or expression of CD27. In some embodiments, the immune checkpoint modulator is an agent that binds to CD27 (e.g., an anti-CD27 antibody). In some embodiments, the checkpoint modulator is a CD27 agonist. In some embodiments, the checkpoint modulator is a CD27 antagonist. In some embodiments, the immune checkpoint modulator is an CD27-binding protein (e.g., an antibody). In some embodiments, the immune checkpoint modulator is varlilumab (Celldex Therapeutics). Additional CD27-binding proteins (e.g., antibodies) are known in the art and are disclosed, e.g., in U.S. Pat. Nos. 9,248,183, 9,102,737, 9,169,325, 9,023,999, 8,481,029; U.S. Patent Application Publication Nos. 2016/0185870, 2015/0337047, 2015/0299330, 2014/0112942, 2013/0336976, 2013/0243795, 2013/0183316, 2012/0213771, 2012/0093805, 2011/0274685, 2010/0173324; and PCT Publication Nos. WO 2015/016718, WO 2014/140374, WO 2013/138586, WO 2012/004367, WO 2011/130434, WO 2010/001908, and WO 2008/051424, each of which is incorporated by reference herein.

CD28. Cluster of Differentiation 28 (CD28) is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular). Binding with its two ligands, CD80 and CD86, expressed on dendritic cells, prompts T cell expansion (see, e.g., Prasad et al. (1994) Proc. Nat'l. Acad. Sci. USA 91(7): 2834-8). Multiple immune checkpoint modulators specific for CD28 have been developed and may be used as disclosed herein. In some embodiments, the immune checkpoint modulator is an agent that modulates the activity and/or expression of CD28. In some embodiments, the immune checkpoint modulator is an agent that binds to CD28 (e.g., an anti-CD28 antibody). In some embodiments, the checkpoint modulator is an CD28 agonist. In some embodiments, the checkpoint modulator is an CD28 antagonist. In some embodiments, the immune checkpoint modulator is an CD28-binding protein (e.g., an antibody). In some embodiments, the immune checkpoint modulator is selected from the group consisting of TAB08 (TheraMab LLC), lulizumab (also known as BMS-931699, Bristol-Myers Squibb), and FR104 (OSE Immunotherapeutics). Additional CD28-binding proteins (e.g., antibodies) are known in the art and are disclosed, e.g., in U.S. Pat. Nos. 9,119,840, 8,709,414, 9,085,629, 8,034,585, 7,939,638, 8,389,016, 7,585,960, 8,454,959, 8,168,759, 8,785,604, 7,723,482; U.S. Patent Application Publication Nos. 2016/0017039, 2015/0299321, 2015/0150968, 2015/0071916, 2015/0376278, 2013/0078257, 2013/0230540, 2013/0078236, 2013/0109846, 2013/0266577, 2012/0201814, 2012/0082683, 2012/0219553, 2011/0189735, 2011/0097339, 2010/0266605, 2010/0168400, 2009/0246204, 2008/0038273; and PCT Publication Nos. WO 2015198147, WO 2016/05421, WO 2014/1209168, WO 2011/101791, WO 2010/007376, WO 2010/009391, WO 2004/004768, WO 2002/030459, WO 2002/051871, and WO 2002/047721, each of which is incorporated by reference herein.

CD40. Cluster of Differentiation 40 (CD40, also known as TNFRSF5) is found on a variety of immune system cells including antigen presenting cells. CD40L, otherwise known as CD154, is the ligand of CD40 and is transiently expressed on the surface of activated CD4⁺ T cells. CD40 signaling is known to ‘license’ dendritic cells to mature and thereby trigger T-cell activation and differentiation (see, e.g., O'Sullivan et al. (2003) Crit. Rev. Immunol. 23 (1): 83-107. Multiple immune checkpoint modulators specific for CD40 have been developed and may be used as disclosed herein. In some embodiments, the immune checkpoint modulator is an agent that modulates the activity and/or expression of CD40. In some embodiments, the immune checkpoint modulator is an agent that binds to CD40 (e.g., an anti-CD40 antibody). In some embodiments, the checkpoint modulator is a CD40 agonist. In some embodiments, the checkpoint modulator is an CD40 antagonist. In some embodiments, the immune checkpoint modulator is a CD40-binding protein selected from the group consisting of dacetuzumab (Genentech/Seattle Genetics), CP-870,893 (Pfizer), bleselumab (Astellas Pharma), lucatumumab (Novartis), CFZ533 (Novartis; see, e.g., Cordoba et al. (2015) Am. J. Transplant. 15(11): 2825-36), RG7876 (Genentech Inc.), FFP104 (PanGenetics, B.V.), APX005 (Apexigen), BI 655064 (Boehringer Ingelheim), Chi Lob 7/4 (Cancer Research UK; see, e.g., Johnson et al. (2015) Clin. Cancer Res. 21(6): 1321-8), ADC-1013 (Biolnvent International), SEA-CD40 (Seattle Genetics), XmAb 5485 (Xencor), PG120 (PanGenetics B.V.), teneliximab (Bristol-Myers Squibb; see, e.g., Thompson et al. (2011) Am. J. Transplant. 11(5): 947-57), and AKH3 (Biogen; see, e.g., International Publication No. WO 2016/028810). Additional CD40-binding proteins (e.g., antibodies) are known in the art and are disclosed, e.g., in U.S. Pat. Nos. 9,234,044, 9,266,956, 9,109,011, 9,090,696, 9,023,360, 9,023,361, 9,221,913, 8,945,564, 8,926,979, 8,828,396, 8,637,032, 8,277,810, 8,088,383, 7,820,170, 7,790,166, 7,445,780, 7,361,345, 8,961,991, 8,669,352, 8,957,193, 8,778,345, 8,591,900, 8,551,485, 8,492,531, 8,362,210, 8,388,971; U.S. Patent Application Publication Nos. 2016/0045597, 2016/0152713, 2016/0075792, 2015/0299329, 2015/0057437 2015/0315282, 2015/0307616, 2014/0099317, 2014/0179907, 2014/0349395, 2014/0234344, 2014/0348836, 2014/0193405, 2014/0120103, 2014/0105907, 2014/0248266, 2014/0093497, 2014/0010812, 2013/0024956, 2013/0023047, 2013/0315900, 2012/0087927, 2012/0263732, 2012/0301488, 2011/0027276, 2011/0104182, 2010/0234578, 2009/0304687, 2009/0181015, 2009/0130715, 2009/0311254, 2008/0199471, 2008/0085531, 2016/0152721, 2015/0110783, 2015/0086991, 2015/0086559, 2014/0341898, 2014/0205602, 2014/0004131, 2013/0011405, 2012/0121585, 2011/0033456, 2011/0002934, 2010/0172912, 2009/0081242, 2009/0130095, 2008/0254026, 2008/0075727, 2009/0304706, 2009/0202531, 2009/0117111, 2009/0041773, 2008/0274118, 2008/0057070, 2007/0098717, 2007/0218060, 2007/0098718, 2007/0110754; and PCT Publication Nos. WO 2016/069919, WO 2016/023960, WO 2016/023875, WO 2016/028810, WO 2015/134988, WO 2015/091853, WO 2015/091655, WO 2014/065403, WO 2014/070934, WO 2014/065402, WO 2014/207064, WO 2013/034904, WO 2012/125569, WO 2012/149356, WO 2012/111762, WO 2012/145673, WO 2011/123489, WO 2010/123012, WO 2010/104761, WO 2009/094391, WO 2008/091954, WO 2007/129895, WO 2006/128103, WO 2005/063289, WO 2005/063981, WO 2003/040170, WO 2002/011763, WO 2000/075348, WO 2013/164789, WO 2012/075111, WO 2012/065950, WO 2009/062054, WO 2007/124299, WO 2007/053661, WO 2007/053767, WO 2005/044294, WO 2005/044304, WO 2005/044306, WO 2005/044855, WO 2005/044854, WO 2005/044305, WO 2003/045978, WO 2003/029296, WO 2002/028481, WO 2002/028480, WO 2002/028904, WO 2002/028905, WO 2002/088186, and WO 2001/024823, each of which is incorporated by reference herein.

CD122. CD122 is the Interleukin-2 receptor beta sub-unit and is known to increase proliferation of CD8⁺ effector T cells. See, e.g., Boyman et al. (2012) Nat. Rev. Immunol. 12 (3): 180-190. Multiple immune checkpoint modulators specific for CD122 have been developed and may be used as disclosed herein. In some embodiments, the immune checkpoint modulator is an agent that modulates the activity and/or expression of CD122. In some embodiments, the immune checkpoint modulator is an agent that binds to CD122 (e.g., an anti-CD122 antibody). In some embodiments, the checkpoint modulator is an CD122 agonist. In some embodiments, the checkpoint modulator is an CD22 agonist. In some embodiments, the immune checkpoint modulator is humanized MiK-Beta-1 (Roche; see, e.g., Morris et al. (2006) Proc Nat'l. Acad. Sci. USA 103(2): 401-6, which is incorporated by reference). Additional CD122-binding proteins (e.g., antibodies) are known in the art and are disclosed, e.g., in U.S. Pat. No. 9,028,830, which is incorporated by reference herein.

OX40. The OX40 receptor (also known as CD134) promotes the expansion of effector and memory T cells. OX40 also suppresses the differentiation and activity of T-regulatory cells, and regulates cytokine production (see, e.g., Croft et al. (2009) Immunol. Rev. 229(1): 173-91). Multiple immune checkpoint modulators specific for OX40 have been developed and may be used as disclosed herein. In some embodiments, the immune checkpoint modulator is an agent that modulates the activity and/or expression of OX40. In some embodiments, the immune checkpoint modulator is an agent that binds to OX40 (e.g., an anti-OX40 antibody). In some embodiments, the checkpoint modulator is an OX40 agonist. In some embodiments, the checkpoint modulator is an OX40 antagonist. In some embodiments, the immune checkpoint modulator is a OX40-binding protein (e.g., an antibody) selected from the group consisting of MEDI6469 (AgonOx/Medimmune), pogalizumab (also known as MOXR0916 and RG7888; Genentech, Inc.), tavolixizumab (also known as MEDI0562; Medimmune), and GSK3174998 (GlaxoSmithKline). Additional OX-40-binding proteins (e.g., antibodies) are known in the art and are disclosed, e.g., in U.S. Pat. Nos. 9,163,085, 9,040,048, 9,006,396, 8,748,585, 8,614,295, 8,551,477, 8,283,450, 7,550,140; U.S. Patent Application Publication Nos. 2016/0068604, 2016/0031974, 2015/0315281, 2015/0132288, 2014/0308276, 2014/0377284, 2014/0044703, 2014/0294824, 2013/0330344, 2013/0280275, 2013/0243772, 2013/0183315, 2012/0269825, 2012/0244076, 2011/0008368, 2011/0123552, 2010/0254978, 2010/0196359, 2006/0281072; and PCT Publication Nos. WO 2014/148895, WO 2013/068563, WO 2013/038191, WO 2013/028231, WO 2010/096418, WO 2007/062245, and WO 2003/106498, each of which is incorporated by reference herein.

GITR. Glucocorticoid-induced TNFR family related gene (GITR) is a member of the tumor necrosis factor receptor (TNFR) superfamily that is constitutively or conditionally expressed on Treg, CD4, and CD8 T cells. GITR is rapidly upregulated on effector T cells following TCR ligation and activation. The human GITR ligand (GITRL) is constitutively expressed on APCs in secondary lymphoid organs and some nonlymphoid tissues. The downstream effect of GITR:GITRL interaction induces attenuation of Treg activity and enhances CD4⁺ T cell activity, resulting in a reversal of Treg-mediated immunosuppression and increased immune stimulation. Multiple immune checkpoint modulators specific for GITR have been developed and may be used as disclosed herein. In some embodiments, the immune checkpoint modulator is an agent that modulates the activity and/or expression of GITR. In some embodiments, the immune checkpoint modulator is an agent that binds to GITR (e.g., an anti-GITR antibody). In some embodiments, the checkpoint modulator is an GITR agonist. In some embodiments, the checkpoint modulator is an GITR antagonist. In some embodiments, the immune checkpoint modulator is a GITR-binding protein (e.g., an antibody) selected from the group consisting of TRX518 (Leap Therapeutics), MK-4166 (Merck & Co.), MEDI-1873 (MedImmune), INCAGN1876 (Agenus/Incyte), and FPA154 (Five Prime Therapeutics). Additional GITR-binding proteins (e.g., antibodies) are known in the art and are disclosed, e.g., in U.S. Pat. Nos. 9,309,321, 9,255,152, 9,255,151, 9,228,016, 9,028,823, 8,709,424, 8,388,967; U.S. Patent Application Publication Nos. 2016/0145342, 2015/0353637, 2015/0064204, 2014/0348841, 2014/0065152, 2014/0072566, 2014/0072565, 2013/0183321, 2013/0108641, 2012/0189639; and PCT Publication Nos. WO 2016/054638, WO 2016/057841, WO 2016/057846, WO 2015/187835, WO 2015/184099, WO 2015/031667, WO 2011/028683, and WO 2004/107618, each of which is incorporated by reference herein.

ICOS. Inducible T-cell costimulator (ICOS, also known as CD278) is expressed on activated T cells. Its ligand is ICOSL, which is expressed mainly on B cells and dendritic cells. ICOS is important in T cell effector function. ICOS expression is up-regulated upon T cell activation (see, e.g., Fan et al. (2014) J. Exp. Med. 211(4): 715-25). Multiple immune checkpoint modulators specific for ICOS have been developed and may be used as disclosed herein. In some embodiments, the immune checkpoint modulator is an agent that modulates the activity and/or expression of ICOS. In some embodiments, the immune checkpoint modulator is an agent that binds to ICOS (e.g., an anti-ICOS antibody). In some embodiments, the checkpoint modulator is an ICOS agonist. In some embodiments, the checkpoint modulator is an ICOS antagonist. In some embodiments, the immune checkpoint modulator is a ICOS-binding protein (e.g., an antibody) selected from the group consisting of MEDI-570 (also known as JMab-136, Medimmune), GSK3359609 (GlaxoSmithKline/INSERM), and JTX-2011 (Jounce Therapeutics). Additional ICOS-binding proteins (e.g., antibodies) are known in the art and are disclosed, e.g., in U.S. Pat. Nos. 9,376,493, 7,998,478, 7,465,445, 7,465,444; U.S. Patent Application Publication Nos. 2015/0239978, 2012/0039874, 2008/0199466, 2008/0279851; and PCT Publication No. WO 2001/087981, each of which is incorporated by reference herein.

4-1BB. 4-1BB (also known as CD137) is a member of the tumor necrosis factor (TNF) receptor superfamily. 4-1BB (CD137) is a type II transmembrane glycoprotein that is inducibly expressed on primed CD4⁺ and CD8⁺ T cells, activated NK cells, DCs, and neutrophils, and acts as a T cell costimulatory molecule when bound to the 4-1BB ligand (4-1BBL) found on activated macrophages, B cells, and DCs. Ligation of the 4-1BB receptor leads to activation of the NF-κB, c-Jun and p38 signaling pathways and has been shown to promote survival of CD8⁺ T cells, specifically, by upregulating expression of the antiapoptotic genes BcL-x(L) and Bfl-1. In this manner, 4-1BB serves to boost or even salvage a suboptimal immune response. Multiple immune checkpoint modulators specific for 4-1BB have been developed and may be used as disclosed herein. In some embodiments, the immune checkpoint modulator is an agent that modulates the activity and/or expression of 4-1BB. In some embodiments, the immune checkpoint modulator is an agent that binds to 4-1BB (e.g., an anti-4-1BB antibody). In some embodiments, the checkpoint modulator is an 4-1BB agonist. In some embodiments, the checkpoint modulator is an 4-1BB antagonist. In some embodiments, the immune checkpoint modulator is a 4-1BB-binding protein is urelumab (also known as BMS-663513; Bristol-Myers Squibb) or utomilumab (Pfizer). In some embodiments, the immune checkpoint modulator is a 4-1BB-binding protein (e.g., an antibody). 4-1BB-binding proteins (e.g., antibodies) are known in the art and are disclosed, e.g., in U.S. Pat. Nos. 9,382,328, 8,716,452, 8,475,790, 8,137,667, 7,829,088, 7,659,384; U.S. Patent Application Publication Nos. 2016/0083474, 2016/0152722, 2014/0193422, 2014/0178368, 2013/0149301, 2012/0237498, 2012/0141494, 2012/0076722, 2011/0177104, 2011/0189189, 2010/0183621, 2009/0068192, 2009/0041763, 2008/0305113, 2008/0008716; and PCT Publication Nos. WO 2016/029073, WO 2015/188047, WO 2015/179236, WO 2015/119923, WO 2012/032433, WO 2012/145183, WO 2011/031063, WO 2010/132389, WO 2010/042433, WO 2006/126835, WO 2005/035584, WO 2004/010947; and Martinez-Forero et al. (2013) J. Immunol. 190(12): 6694-706, and Dubrot et al. (2010) Cancer Immunol. Immunother. 59(8): 1223-33, each of which is incorporated by reference herein.

### Inhibitory Immune Checkpoint Molecules

ADORA2A. The adenosine A2A receptor (A2A4) is a member of the G protein-coupled receptor (GPCR) family which possess seven transmembrane alpha helices, and is regarded as an important checkpoint in cancer therapy. A2A receptor can negatively regulate overreactive immune cells (see, e.g., Ohta et al. (2001) Nature 414(6866): 916-20). Multiple immune checkpoint modulators specific for ADORA2A have been developed and may be used as disclosed herein. In some embodiments, the immune checkpoint modulator is an agent that modulates the activity and/or expression of ADORA2A. In some embodiments, the immune checkpoint modulator is an agent that binds to ADORA2A (e.g., an anti-ADORA2A antibody). In some embodiments, the immune checkpoint modulator is a ADORA2A-binding protein (e.g., an antibody). In some embodiments, the checkpoint modulator is an ADORA2A agonist. In some embodiments, the checkpoint modulator is an ADORA2A antagonist. ADORA2A-binding proteins (e.g., antibodies) are known in the art and are disclosed, e.g., in U.S. Patent Application Publication No. 2014/0322236, which is incorporated by reference herein.

B7-H3. B7-H3 (also known as CD276) belongs to the B7 superfamily, a group of molecules that costimulate or down-modulate T-cell responses. B7-H3 potently and consistently down-modulates human T-cell responses (see, e.g., Leitner et al. (2009) Eur. J. Immunol. 39(7): 1754-64). Multiple immune checkpoint modulators specific for B7-H3 have been developed and may be used as disclosed herein. In some embodiments, the immune checkpoint modulator is an agent that modulates the activity and/or expression of B7-H3. In some embodiments, the immune checkpoint modulator is an agent that binds to B7-H3 (e.g., an anti-B7-H3 antibody). In some embodiments, the checkpoint modulator is an B7-H3 agonist. In some embodiments, the checkpoint modulator is an B7-H3 antagonist. In some embodiments, the immune checkpoint modulator is an anti-B7-H3-binding protein selected from the group consisting of DS-5573 (Daiichi Sankyo, Inc.), enoblituzumab (MacroGenics, Inc.), and 8H9 (Sloan Kettering Institute for Cancer Research; see, e.g., Ahmed et al. (2015) J. Biol. Chem. 290(50): 30018-29). In some embodiments, the immune checkpoint modulator is a B7-H3-binding protein (e.g., an antibody). B7-H3-binding proteins (e.g., antibodies) are known in the art and are disclosed, e.g., in U.S. Pat. Nos. 9,371,395, 9,150,656, 9,062,110, 8,802,091, 8,501,471, 8,414,892; U.S. Patent Application Publication Nos. 2015/0352224, 2015/0297748, 2015/0259434, 2015/0274838, 2014/032875, 2014/0161814, 2013/0287798, 2013/0078234, 2013/0149236, 2012/02947960, 2010/0143245, 2002/0102264; PCT Publication Nos. WO 2016/106004, WO 2016/033225, WO 2015/181267, WO 2014/057687, WO 2012/147713, WO 2011/109400, WO 2008/116219, WO 2003/075846, WO 2002/032375; and Shi et al. (2016) Mol. Med. Rep. 14(1): 943-8, each of which is incorporated by reference herein.

B7-H4. B7-H4 (also known as 08E, OV064, and V-set domain-containing T-cell activation inhibitor (VTCN1)), belongs to the B7 superfamily. By arresting cell cycle, B7-H4 ligation of T cells has a profound inhibitory effect on the growth, cytokine secretion, and development of cytotoxicity. Administration of B7-H4Ig into mice impairs antigen-specific T cell responses, whereas blockade of endogenous B7-H4 by specific monoclonal antibody promotes T cell responses (see, e.g., Sica et al. (2003) Immunity 18(6): 849-61). Multiple immune checkpoint modulators specific for B7-H4 have been developed and may be used as disclosed herein. In some embodiments, the immune checkpoint modulator is an agent that modulates the activity and/or expression of B7-H4. In some embodiments, the immune checkpoint modulator is an agent that binds to B7-H4 (e.g., an anti-B7-H4 antibody). In some embodiments, the immune checkpoint modulator is a B7-H4-binding protein (e.g., an antibody). In some embodiments, the checkpoint modulator is an B7-H4 agonist. In some embodiments, the checkpoint modulator is an B7-H4 antagonist. B7-H4-binding proteins (e.g., antibodies) are known in the art and are disclosed, e.g., in U.S. Pat. Nos. 9,296,822, 8,609,816, 8,759,490, 8,323,645; U.S. Patent Application Publication Nos. 2016/0159910, 2016/0017040, 2016/0168249, 2015/0315275, 2014/0134180, 2014/0322129, 2014/0356364, 2014/0328751, 2014/0294861, 2014/0308259, 2013/0058864, 2011/0085970, 2009/0074660, 2009/0208489; and PCT Publication Nos. WO 2016/040724, WO 2016/070001, WO 2014/159835, WO 2014/100483, WO 2014/100439, WO 2013/067492, WO 2013/025779, WO 2009/073533, WO 2007/067991, and WO 2006/104677, each of which is incorporated by reference herein.

BTLA. B and T Lymphocyte Attenuator (BTLA), also known as CD272, has HVEM (Herpesvirus Entry Mediator) as its ligand. Surface expression of BTLA is gradually downregulated during differentiation of human CD8⁺ T cells from the naive to effector cell phenotype, however tumor-specific human CD8⁺ T cells express high levels of BTLA (see, e.g., Derre et al. (2010) J. Clin. Invest. 120 (1): 157-67). Multiple immune checkpoint modulators specific for BTLA have been developed and may be used as disclosed herein. In some embodiments, the immune checkpoint modulator is an agent that modulates the activity and/or expression of BTLA. In some embodiments, the immune checkpoint modulator is an agent that binds to BTLA (e.g., an anti-BTLA antibody). In some embodiments, the immune checkpoint modulator is a BTLA-binding protein (e.g., an antibody). In some embodiments, the checkpoint modulator is an BTLA agonist. In some embodiments, the checkpoint modulator is an BTLA antagonist. BTLA-binding proteins (e.g., antibodies) are known in the art and are disclosed, e.g., in U.S. Pat. Nos. 9,346,882, 8,580,259, 8,563,694, 8,247,537; U.S. Patent Application Publication Nos. 2014/0017255, 2012/0288500, 2012/0183565, 2010/0172900; and PCT Publication Nos. WO 2011/014438, and WO 2008/076560, each of which is incorporated by reference herein.

CTLA-4. Cytotoxic T lymphocyte antigen-4 (CTLA-4) is a member of the immune regulatory CD28-B7 immunoglobulin superfamily and acts on naïve and resting T lymphocytes to promote immunosuppression through both B7-dependent and B7-independent pathways (see, e.g., Kim et al. (2016) J. Immunol. Res., Article ID 4683607, 14 pp.). CTLA-4 is also known as called CD152. CTLA-4 modulates the threshold for T cell activation. See, e.g., Gajewski et al. (2001) J. Immunol. 166(6): 3900-7. Multiple immune checkpoint modulators specific for CTLA-4 have been developed and may be used as disclosed herein. In some embodiments, the immune checkpoint modulator is an agent that modulates the activity and/or expression of CTLA-4. In some embodiments, the immune checkpoint modulator is an agent that binds to CTLA-4 (e.g., an anti-CTLA-4 antibody). In some embodiments, the checkpoint modulator is an CTLA-4 agonist. In some embodiments, the checkpoint modulator is an CTLA-4 antagonist. In some embodiments, the immune checkpoint modulator is a CTLA-4-binding protein (e.g., an antibody) selected from the group consisting of ipilimumab (Yervoy; Medarex/Bristol-Myers Squibb), tremelimumab (formerly ticilimumab; Pfizer/AstraZeneca), JMW-3B3 (University of Aberdeen), and AGEN1884 (Agenus). Additional CTLA-4 binding proteins (e.g., antibodies) are known in the art and are disclosed, e.g., in U.S. Pat. No. 8,697,845; U.S. Patent Application Publication Nos. 2014/0105914, 2013/0267688, 2012/0107320, 2009/0123477; and PCT Publication Nos. WO 2014/207064, WO 2012/120125, WO 2016/015675, WO 2010/097597, WO 2006/066568, and WO 2001/054732, each of which is incorporated by reference herein.

IDO. Indoleamine 2,3-dioxygenase (IDO) is a tryptophan catabolic enzyme with immune-inhibitory properties. Another important molecule is TDO, tryptophan 2,3-dioxygenase. IDO is known to suppress T and NK cells, generate and activate Tregs and myeloid-derived suppressor cells, and promote tumor angiogenesis. Prendergast et al., 2014, Cancer Immunol Immunother. 63 (7): 721-35, which is incorporated by reference herein.

Multiple immune checkpoint modulators specific for IDO have been developed and may be used as disclosed herein. In some embodiments, the immune checkpoint modulator is an agent that modulates the activity and/or expression of IDO. In some embodiments, the immune checkpoint modulator is an agent that binds to IDO (e.g., an IDO binding protein, such as an anti-IDO antibody). In some embodiments, the checkpoint modulator is an IDO agonist. In some embodiments, the checkpoint modulator is an IDO antagonist. In some embodiments, the immune checkpoint modulator is selected from the group consisting of Norharmane, Rosmarinic acid, COX-2 inhibitors, alpha-methyl-tryptophan, and Epacadostat. In one embodiment, the modulator is Epacadostat.

KIR. Killer immunoglobulin-like receptors (KIRs) comprise a diverse repertoire of MHCI binding molecules that negatively regulate natural killer (NK) cell function to protect cells from NK-mediated cell lysis. KIRs are generally expressed on NK cells but have also been detected on tumor specific CTLs. Multiple immune checkpoint modulators specific for KIR have been developed and may be used as disclosed herein. In some embodiments, the immune checkpoint modulator is an agent that modulates the activity and/or expression of KIR. In some embodiments, the immune checkpoint modulator is an agent that binds to KIR (e.g., an anti-KIR antibody). In some embodiments, the immune checkpoint modulator is a KIR-binding protein (e.g., an antibody). In some embodiments, the checkpoint modulator is an KIR agonist. In some embodiments, the checkpoint modulator is an KIR antagonist. In some embodiments the immune checkpoint modulator is lirilumab (also known as BMS-986015; Bristol-Myers Squibb). Additional KIR binding proteins (e.g., antibodies) are known in the art and are disclosed, e.g., in U.S. Pat. Nos. 8,981,065, 9,018,366, 9,067,997, 8,709,411, 8,637,258, 8,614,307, 8,551,483, 8,388,970, 8,119,775; U.S. Patent Application Publication Nos. 2015/0344576, 2015/0376275, 2016/0046712, 2015/0191547, 2015/0290316, 2015/0283234, 2015/0197569, 2014/0193430, 2013/0143269, 2013/0287770, 2012/0208237, 2011/0293627, 2009/0081240, 2010/0189723; and PCT Publication Nos. WO 2016/069589, WO 2015/069785, WO 2014/066532, WO 2014/055648, WO 2012/160448, WO 2012/071411, WO 2010/065939, WO 2008/084106, WO 2006/072625, WO 2006/072626, and WO 2006/003179, each of which is incorporated by reference herein.

LAG-3, Lymphocyte-activation gene 3 (LAG-3, also known as CD223) is a CD4-related transmembrane protein that competitively binds MHC II and acts as a co-inhibitory checkpoint for T cell activation (see, e.g., Goldberg and Drake (2011) Curr. Top. Microbiol. Immunol. 344: 269-78). Multiple immune checkpoint modulators specific for LAG-3 have been developed and may be used as disclosed herein. In some embodiments, the immune checkpoint modulator is an agent that modulates the activity and/or expression of LAG-3. In some embodiments, the immune checkpoint modulator is an agent that binds to LAG-3 (e.g., an anti-PD-1 antibody). In some embodiments, the checkpoint modulator is an LAG-3 agonist. In some embodiments, the checkpoint modulator is an LAG-3 antagonist. In some embodiments, the immune checkpoint modulator is a LAG-3-binding protein (e.g., an antibody) selected from the group consisting of pembrolizumab (Keytruda; formerly lambrolizumab; Merck & Co., Inc.), nivolumab (Opdivo; Bristol-Myers Squibb), pidilizumab (CT-011, CureTech), SHR-1210 (Incyte/Jiangsu Hengrui Medicine Co., Ltd.), MEDI0680 (also known as AMP-514; Amplimmune Inc./Medimmune), PDR001 (Novartis), BGB-A317 (BeiGene Ltd.), TSR-042 (also known as ANBO11; AnaptysBio/Tesaro, Inc.), REGN2810 (Regeneron Pharmaceuticals, Inc./Sanofi-Aventis), and PF-06801591 (Pfizer). Additional PD-1-binding proteins (e.g., antibodies) are known in the art and are disclosed, e.g., in U.S. Pat. Nos. 9,181,342, 8,927,697, 7,488,802, 7,029,674; U.S. Patent Application Publication Nos. 2015/0152180, 2011/0171215, 2011/0171220; and PCT Publication Nos. WO 2004/056875, WO 2015/036394, WO 2010/029435, WO 2010/029434, WO 2014/194302, each of which is incorporated by reference herein.

PD-1. Programmed cell death protein 1 (PD-1, also known as CD279 and PDCD1) is an inhibitory receptor that negatively regulates the immune system. In contrast to CTLA-4 which mainly affects naïve T cells, PD-1 is more broadly expressed on immune cells and regulates mature T cell activity in peripheral tissues and in the tumor microenvironment. PD-1 inhibits T cell responses by interfering with T cell receptor signaling. PD-1 has two ligands, PD-L1 and PD-L2. Multiple immune checkpoint modulators specific for PD-1 have been developed and may be used as disclosed herein. In some embodiments, the immune checkpoint modulator is an agent that modulates the activity and/or expression of PD-1. In some embodiments, the immune checkpoint modulator is an agent that binds to PD-1 (e.g., an anti-PD-1 antibody). In some embodiments, the checkpoint modulator is an PD-1 agonist. In some embodiments, the checkpoint modulator is an PD-1 antagonist. In some embodiments, the immune checkpoint modulator is a PD-1-binding protein (e.g., an antibody) selected from the group consisting of pembrolizumab (Keytruda; formerly lambrolizumab; Merck & Co., Inc.), nivolumab (Opdivo; Bristol-Myers Squibb), pidilizumab (CT-011, CureTech), SHR-1210 (Incyte/Jiangsu Hengrui Medicine Co., Ltd.), MEDI0680 (also known as AMP-514; Amplimmune Inc./Medimmune), PDR001 (Novartis), BGB-A317 (BeiGene Ltd.), TSR-042 (also known as ANBO11; AnaptysBio/Tesaro, Inc.), REGN2810 (Regeneron Pharmaceuticals, Inc./Sanofi-Aventis), and PF-06801591 (Pfizer). Additional PD-1-binding proteins (e.g., antibodies) are known in the art and are disclosed, e.g., in U.S. Pat. Nos. 9,181,342, 8,927,697, 7,488,802, 7,029,674; U.S. Patent Application Publication Nos. 2015/0152180, 2011/0171215, 2011/0171220; and PCT Publication Nos. WO 2004/056875, WO 2015/036394, WO 2010/029435, WO 2010/029434, WO 2014/194302, each of which is incorporated by reference herein.

PD-L1/PD-L2. PD ligand 1 (PD-L1, also knows as B7-H1) and PD ligand 2 (PD-L2, also known as PDCD1LG2, CD273, and B7-DC) bind to the PD-1 receptor. Both ligands belong to the same B7 family as the B7-1 and B7-2 proteins that interact with CD28 and CTLA-4. PD-L1 can be expressed on many cell types including, for example, epithelial cells, endothelial cells, and immune cells. Ligation of PDL-1 decreases IFNγ, TNFα, and IL-2 production and stimulates production of IL10, an anti-inflammatory cytokine associated with decreased T cell reactivity and proliferation as well as antigen-specific T cell anergy. PDL-2 is predominantly expressed on antigen presenting cells (APCs). PDL2 ligation also results in T cell suppression, but where PDL-1-PD-1 interactions inhibits proliferation via cell cycle arrest in the G1/G2 phase, PDL2-PD-1 engagement has been shown to inhibit TCR-mediated signaling by blocking B7:CD28 signals at low antigen concentrations and reducing cytokine production at high antigen concentrations. Multiple immune checkpoint modulators specific for PD-L1 and PD-L2 have been developed and may be used as disclosed herein.

In some embodiments, the immune checkpoint modulator is an agent that modulates the activity and/or expression of PD-L1. In some embodiments, the immune checkpoint modulator is an agent that binds to PD-L1 (e.g., an anti-PD-L1 antibody). In some embodiments, the checkpoint modulator is an PD-L1 agonist. In some embodiments, the checkpoint modulator is an PD-L1 antagonist. In some embodiments, the immune checkpoint modulator is a PD-L1-binding protein (e.g., an antibody or a Fc-fusion protein) selected from the group consisting of durvalumab (also known as MEDI-4736; AstraZeneca/Celgene Corp./Medimmune), atezolizumab (Tecentriq; also known as MPDL3280A and RG7446; Genetech Inc.), avelumab (also known as MSB0010718C; Merck Serono/AstraZeneca); MDX-1105 (Medarex/Bristol-Meyers Squibb), AMP-224 (Amplimmune, GlaxoSmithKline), LY3300054 (Eli Lilly and Co.). Additional PD-L1-binding proteins are known in the art and are disclosed, e.g., in U.S. Patent Application Publication Nos. 2016/0084839, 2015/0355184, 2016/0175397, and PCT Publication Nos. WO 2014/100079, WO 2016/030350, WO2013181634, each of which is incorporated by reference herein.

In some embodiments, the immune checkpoint modulator is an agent that modulates the activity and/or expression of PD-L2. In some embodiments, the immune checkpoint modulator is an agent that binds to PD-L2 (e.g., an anti-PD-L2 antibody). In some embodiments, the checkpoint modulator is an PD-L2 agonist. In some embodiments, the checkpoint modulator is an PD-L2 antagonist. PD-L2-binding proteins (e.g., antibodies) are known in the art and are disclosed, e.g., in U.S. Pat. Nos. 9,255,147, 8,188,238; U.S. Patent Application Publication Nos. 2016/0122431, 2013/0243752, 2010/0278816, 2016/0137731, 2015/0197571, 2013/0291136, 2011/0271358; and PCT Publication Nos. WO 2014/022758, and WO 2010/036959, each of which is incorporated by reference herein.

TIM-3. T cell immunoglobulin mucin 3 (TIM-3, also known as Hepatitis A virus cellular receptor (HAVCR2)) is a A type I glycoprotein receptor that binds to S-type lectin galectin-9 (Gal-9). TIM-3, is a widely expressed ligand on lymphocytes, liver, small intestine, thymus, kidney, spleen, lung, muscle, reticulocytes, and brain tissue. Tim-3 was originally identified as being selectively expressed on IFN-7-secreting Th1 and Tcl cells (Monney et al. (2002) Nature 415: 536-41). Binding of Gal-9 by the TIM-3 receptor triggers downstream signaling to negatively regulate T cell survival and function. Multiple immune checkpoint modulators specific for TIM-3 have been developed and may be used as disclosed herein. In some embodiments, the immune checkpoint modulator is an agent that modulates the activity and/or expression of TIM-3. In some embodiments, the immune checkpoint modulator is an agent that binds to TIM-3 (e.g., an anti-TIM-3 antibody). In some embodiments, the checkpoint modulator is an TIM-3 agonist. In some embodiments, the checkpoint modulator is an TIM-3 antagonist. In some embodiments, the immune checkpoint modulator is an anti-TIM-3 antibody selected from the group consisting of TSR-022 (AnaptysBio/Tesaro, Inc.) and MGB453 (Novartis). Additional TIM-3 binding proteins (e.g., antibodies) are known in the art and are disclosed, e.g., in U.S. Pat. Nos. 9,103,832, 8,552,156, 8,647,623, 8,841,418; U.S. Patent Application Publication Nos. 2016/0200815, 2015/0284468, 2014/0134639, 2014/0044728, 2012/0189617, 2015/0086574, 2013/0022623; and PCT Publication Nos. WO 2016/068802, WO 2016/068803, WO 2016/071448, WO 2011/155607, and WO 2013/006490, each of which is incorporated by reference herein.

VISTA. V-domain Ig suppressor of T cell activation (VISTA, also known as Platelet receptor Gi24) is an Ig super-family ligand that negatively regulates T cell responses. See, e.g., Wang et al., 2011, J. Exp. Med. 208: 577-92. VISTA expressed on APCs directly suppresses CD4+ and CD8⁺ T cell proliferation and cytokine production (Wang et al. (2010) J Exp Med. 208(3): 577-92). Multiple immune checkpoint modulators specific for VISTA have been developed and may be used as disclosed herein. In some embodiments, the immune checkpoint modulator is an agent that modulates the activity and/or expression of VISTA. In some embodiments, the immune checkpoint modulator is an agent that binds to VISTA (e.g., an anti-VISTA antibody). In some embodiments, the checkpoint modulator is an VISTA agonist. In some embodiments, the checkpoint modulator is an VISTA antagonist. In some embodiments, the immune checkpoint modulator is a VISTA-binding protein (e.g., an antibody) selected from the group consisting of TSR-022 (AnaptysBio/Tesaro, Inc.) and MGB453 (Novartis). VISTA-binding proteins (e.g., antibodies) are known in the art and are disclosed, e.g., in U.S. Patent Application Publication Nos. 2016/0096891, 2016/0096891; and PCT Publication Nos. WO 2014/190356, WO 2014/197849, WO 2014/190356 and WO 2016/094837, each of which is incorporated by reference herein.

### III. Coenzyme 010 Compounds

It will be understood that all of the methods provided in the instant invention may involve administration of, in place of Coenzyme Q10, any other Coenzyme Q10 compound, or a combination thereof. Coenzyme Q10 compounds are intended to include a class of CoQ10 compounds. Coenzyme Q10 compounds effective for the methods described herein include CoQ10, a metabolite of CoQ10, a biosynthetic precursor of CoQ10, an analog of CoQ10, a derivative of CoQ10, and CoQ10 related compounds. An analog of CoQ10 includes analogs having no or at least one isoprenyl repeats. CoQ10 has the following structure:

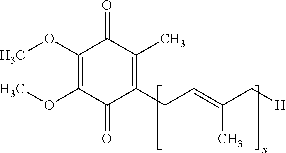

wherein x is 10. In the instant invention, CoQ10 compounds can include derivatives of CoQ10 in which x is any number of isoprenyl units from 4-10, or any number of isoprenyl units from 6-10, or any number of isoprenyl units from 8-10, or 9-10 isoprenyl units. CoQ10 includes the fully oxidized version, also known as ubiquinone, the partially oxidized version, also known as semiquinone or ubisemiquinone, or the fully reduced version, also known as ubiquinol; or any mixtures or combinations thereof. In certain embodiments, the CoQ10 compound for treatment of cancer is ubiquinone. In certain embodiments, the CoQ10 compound for treatment of cancer is ubiquinol.

In certain embodiments of the present invention, the therapeutic agent is Coenzyme Q10 (CoQ10). Coenzyme Q10, also referred to herein as CoQ10, is also known as ubiquinone, or ubidecarenone. CoQ10 is art-recognized and further described in International Publication No. WO 2005/069916 (Appln. No. PCT/US2005/001581), WO 2008/116135 (Appln. No. PCT/US08/57786), WO2010/132507 (Appln. No. PCT/US2010/034453), WO 2011/112900 (Appln. No. PCT/US2011/028042), and WO2012/174559 (Appln. No. PCT/US2012/043001) the entire contents of each of which are expressly incorporated by reference herein. CoQ10 is one of a series of polyprenyl 2,3-dimethoxy-5-methylbenzoquinone (ubiquinone) present in the mitochondrial electron transport systems of eukaryotic cells. Human cells produce CoQ10 exclusively and it is found in cell and mitochondrial membranes of all human cells, with the highest levels in organs with high energy requirements, such as the liver and the heart. The body pool of CoQ10 has been estimated to be about 2 grams, of which more than 50% is endogenous. Approximately 0.5 grams of CoQ10 is required from the diet or biosynthesis each day. CoQ10 is produced in ton quantities from the worldwide supplement market and can be obtained from Kaneka, with plants in Pasadena, Texas and Takasagoshi, Japan.

Coenzyme Q10 related compounds include, but are not limited to, benzoquinones, isoprenoids, farnesols, farnesyl acetate, farnesyl pyrophosphate, 1-phenylalanine, d-phenylalanine, dl-phenylalanine, 1-tyrosine, d-tyrosine, dl-tyrosine, 4-hydroxy-phenylpyruvate, 4-hydroxy-phenyllactate, 4-hydroxy-cinnamate, dipeptides and tripeptides of tyrosine or phenylalanine, 3,4-dihydroxymandelate, 3-methoxy-4-hydroxyphenylglycol, 3-methoxy-4-hydroxymandelate, vanillic acid, phenylacetate, pyridoxine, S-adenosyl methionine, panthenol, mevalonic acid, isopentyl pyrophosphate, phenylbutyrate, 4-hydroxy-benzoate,decaprenyl pyrophosphate, beta-hydroxybutyrate, 3-hydroxy-3-methyl-glutarate, acetylcarnitine, acetoacetylcarnitine, acetylglycine, acetoacetylglycine, carnitine, acetic acid, pyruvic acid, 3-hydroxy-3-methylglutarylcarnitine, all isomeric forms of serine, alanine, cysteine, glycine, threonine, hydroxyproline, lysine, isoleucine, and leucine, even carbon number C4 to C8 fatty acids (butyric, caproic, caprylic, capric, lauric, myristic, palmitic, and stearic acids) salts of carnitine and glycine, e.g., palmitoylcarnitine and palmitoylglycine, and 4-hydroxy-benzoate polyprenyltransferase, any salts of these compounds, as well as any combinations thereof, and the like. In certain embodiments, such agents can be used for the treatment of a cancer according to the methods provided herein.

Metabolites and biosynthetic precursors of CoQ10 include, but are not limited to, those compounds that are formed between the chemical/biological conversion of tyrosine and acetyl-CoA to ubiquinol. Intermediates of the coenzyme biosynthesis pathway include tyrosine, acetyl-CoA, 3-hexaprenyl-4-hydroxybenzoate, 3-hexaprenyl-4,5-dihydroxybenzoate, 3-hexaprenyl-4-hydroxy-5-methoxybenzoate, 2-hexaprenyl-6-methoxy-1,4-benzoquinone, 2-hexaprenyl-3-methyl-6-methoxy-1,4-benzoquinone, 2-hexaprenyl-3-methyl-5-hydroxy-6-methoxy-1,4-benzoquinone, 3-Octaprenyl-4-hydroxybenzoate, 2-octaprenylphenol, 2-octaprenyl-6-metholxyphenol, 2-octaprenyl-3-methyl-6-methoxy-1,4-benzoquinone, 2-octaprenyl-3-methyl-5-hydroxy-6-methoxy-1,4-benzoquinone, 2-decaprenyl-3-methyl-5-hydroxy-6-methoxy-1,4-benzoquinone, 2-decaprenyl-3-methyl-6-methoxy-1,4-benzoquinone, 2-decaprenyl-6-methoxy-1,4-benzoquinone, 2-decaprenyl-6-methoxyphenol, 3-decaprenyl-4-hydroxy-5-methoxybenzoate, 3-decaprenyl-4,5-dihydroxybenzoate, 3-decaprenyl-4-hydroxybenzoate, 4-hydroxy phenylpyruvate, 4-hydroxyphenyllactate, 4-hydroxy-benzoate, 4-hydroxycinnamate, and hexaprenydiphosphate. In certain embodiments, such agents can be used for the treatment of a cancer according to the methods provided herein.

### IV. Compositions

The present disclosure provides compositions containing a CoQ10 compound, e.g., Coenzyme Q10, for the treatment and prevention of cancer. The compositions of the present disclosure can be administered to a patient either by themselves, or in pharmaceutical compositions where it is mixed with suitable carriers or excipient(s). In treating a patient exhibiting an oncological disorder, a therapeutically effective amount of the CoQ10 compound is administered.

Suitable routes of administration of the present compositions of the invention may include parenteral delivery, including, intravenous, intramuscular, subcutaneous, intramedullary injections, as well as intrathecal, direct intraventricular, intraperitoneal, intranasal, or intraocular injections, just to name a few. In one embodiment, the compositions provided herein may be administered by injecting directly to a tumor. In some embodiments, the formulations of the invention may be administered by intravenous injection or intravenous infusion. In some embodiments, the formulation is administered by continuous infusion. In one embodiment, the compositions of the invention are administered by intravenous injection. In one embodiment, the compositions of the invention are administered by intravenous infusion. Where the route of administration is, for example intravenous infusion, embodiments are provided herein where the IV infusion comprises the active agent, e.g., CoQ10, at approximately a 40 mg/mL concentration. Where the composition is administered by IV infusion, it can be diluted in a pharmaceutically acceptable aqueous solution such as phosphate buffered saline or normal saline. In some embodiments, one or more routes of administration may be combined, such as, for example, intravenous and intratumoral, or intravenous and peroral, or intravenous and oral, or intravenous and topical, transdermal, or transmucosal.

The compositions described herein may be administered to a subject in any suitable formulation. These include, for example, liquid, semi-solid, and solid dosage forms, such as liquid solutions (e.g., injectable and infusible solutions), dispersions or suspensions, tablets, pills, powders, creams, lotions, liniments, ointments, or pastes, drops for administration to the eye, ear or nose, liposomes, and suppositories. The preferred form depends on the intended mode of administration and therapeutic application.

In certain embodiments, a CoQ10 compound, e.g., CoQ10, may be prepared with a carrier that will protect against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

For example, a CoQ10 compound e.g., CoQ10, can be formulated for parenteral delivery, e.g., for subcutaneous, intravenous, intramuscular, or intratumoral injection. The compositions may be administered in a single bolus, multiple injections, or by continuous infusion (for example, intravenously or by peritoneal dialysis). For parenteral administration, the compositions may be formulated in a sterilized pyrogen-free form.

Use of pharmaceutically acceptable carriers to formulate the compounds herein disclosed, e.g., Coenzyme Q10 compounds or immune checkpoint modulators, for the practice of the present invention, into dosages suitable for systemic administration is within the scope of the present disclosure. With proper choice of carrier and suitable manufacturing practice, the compositions of the present disclosure, in particular, those formulated as solutions, may be administered parenterally, such as by intravenous injection.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices may be desirable. The data obtained from these cell culture assays and animal studies can be used in formulating a range of dosage for use in human. The dosage of such compounds may be within a range of circulating concentrations that include the ED50 with little or no toxicity. The dosage may vary within this range depending upon the dosage form employed and the route of administration utilized.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers including excipients and auxiliaries which facilitate processing of the active compounds into preparations which can be used pharmaceutically. The preparations formulated for intravenous administration may be in the form of solutions of colloidal dispersion.

Pharmaceutical compositions for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

### V. Formulations

The active agent, e.g., a CoQ10 compound, e.g., CoQ10, can be delivered in any pharmaceutically acceptable carrier for the desired route of administration. As used herein, formulations including CoQ10 compounds are formulated for any route of administration unless otherwise clearly indicated. In preferred embodiments, the formulations are for administration by injection, infusion, or topical administration. In certain embodiments, the CoQ10 compounds are not delivered orally.

Preferred therapeutic formulations for use in the methods of the invention comprise the active agent (e.g., a CoQ10 compound, e.g., CoQ10) in a microparticle formation, e.g., for intravenous administration. Such intravenous formulations are provided, for example, in WO2011/112900 (Appln. No. PCT/US2011/028042), the entire contents of which are expressly incorporated herein by reference, and an exemplary intravenous formulation as described in WO2011/112900 (Appln. No. PCT/US2011/028042) is used in the examples set forth below. Through high pressure homogenization, active agent (e.g., a CoQ10 compound, e.g., CoQ10) particles are reduced to produce particles that are small enough to pass through a 200-nm sterilizing filter. Particles that are small enough to pass through a 200-nm sterilizing filter can be injected intravenously. These particles are much smaller than blood cells and therefore will not embolize capillaries. Red blood cells for example are 6-micron×2-micron disks. The particles are dispersed to and are encased or surrounded by a stabilizing agent. While not wishing to be bound by any theory, it is believed that the stabilizing agents are attracted to the hydrophobic therapeutic agent such that the dispersed particles of the hydrophobic therapeutic agent are surrounded by the stabilizing agent forming a suspension or an emulsion. The dispersed particles in the suspension or emulsion comprises a stabilizing agent surface and a core consisting of the hydrophobic therapeutic agent, e.g., a CoQ10 compound, e.g., CoQ10, in a solid particulate form (suspension) or in an immiscible liquid form (emulsion). The dispersed particles can be entrenched in the lipophilic regions of a liposome.

Dispersed colloidal systems permit a high drug load in the formulation without the use of co-solvents. Additionally, high and relatively reproducible plasma levels are achieved without the dependence on endogenous low-density lipoprotein carriers. More importantly, the formulations allow sustained high drug levels in solid tumors due to the passive accumulation of the colloidal particles of the hydrophobic therapeutic agent.

A preferred intravenous formulation substantially comprises a continuous phase of water and dispersed solids (suspension) or dispersed immiscible liquid (emulsion). Dispersed colloidal systems, in which the particles are composed largely of the active agent (drug) itself, can often deliver more drug per unit volume than continuous solubilizing systems, if the system can be made adequately stable.

As the formulation medium, the aqueous solution may include Hank's solution, Ringer's solution, phosphate buffered saline (PBS), physiological saline buffer or other suitable salts or combinations to achieve the appropriate pH and osmolarity for parenterally delivered formulations. Aqueous solutions can be used to dilute the formulations for administration to the desired concentration. For example, aqueous solutions can be used to dilute a formulation for intravenous administration from a concentration of about 4% w/v to a lower concentration to facilitate administration of lower doses of CoQ10. The aqueous solution may contain substances which increase the viscosity of the solution, such as sodium carboxymethyl cellulose, sorbitol, or dextran.

The active agent (e.g., a CoQ10 compound, e.g., CoQ10) is dispersed in the aqueous solution such that a colloidal dispersion is formed wherein the nano-dispersion particles of the hydrophobic therapeutic agent are covered or encased or encircled by the dispersion stabilizing agents to form nano-dispersions of the active agent (e.g., a CoQ10 compound, e.g., CoQ10) particles. The nano-dispersed active agent (e.g., a CoQ10 compound, e.g., CoQ10) particles have a core formed of the hydrophobic therapeutic agent that is surrounded by the stabilizing agent. Similarly, in certain aspects, the stabilizing agent is a phospholipid having both a hydrophilic and lipophilic portion. The phospholipids form liposomes or other nanoparticles upon homogenization. In certain aspects these liposomes are bi-layered unilamellar liposomes while in other embodiments the liposomes are bi-layered multi-lamellar liposomes. The dispersed active agent (e.g., a CoQ10 compound, e.g., CoQ10) particles are dispersed in the lipophilic portion of the bi-layered structure of the liposome formed from the phospholipids. In certain other aspects the core of the liposome, like the core of the nano-dispersion of active agent (e.g., a CoQ10 compound, e.g., CoQ10) particles, is formed of the hydrophobic therapeutic agent and the outer layer is formed of the bi-layered structure of the phospholipid. In certain embodiments the colloidal dispersions are treated by a lyophilization process whereby the nanoparticle dispersion is converted to a dry powder.

In some embodiments, the formulation for injection or infusion used is a 4% sterile aqueous colloidal dispersion containing CoQ10 in a nanosuspension as prepared in WO2011/112900. In certain embodiments, the formulation includes an aqueous solution; a hydrophobic active agent, e.g., CoQ10, a CoQ10 precursor or metabolite or a CoQ10 related compound, dispersed to form a colloidal nano-dispersion of particles; and at least one of a dispersion stabilizing agent and an opsonization reducer; wherein the colloidal nano-dispersion of the active agent is dispersed into nano-dispersion particles having a mean size of less than 200-nm.

In certain embodiments, the dispersion stabilizing agent includes, but is not limited to, pegylated castor oil, Cremphor® EL, Cremophor® RH 40, Pegylated vitamin E, Vitamin E TPGS, and Dimyristoylphosphatidyl choline (DMPC).

In certain embodiments, the opsonization reducer is a poloxamer or a poloxamines.

In certain embodiments, the colloidal nano-dispersion is a suspension or an emulsion. Optionally, a colloidal nano-dispersion is in a crystalline form or a super-cooled melt form.

In certain embodiments, the formulation for injection or infusion includes a lyoprotectant such as a nutritive sugar including, but not limited to, lactose, mannose, maltose, galactose, fructose, sorbose, raffinose, neuraminic acid, glucosamine, galactosamine, N-methylglucosamine, mannitol, sorbitol, arginine, glycine and sucrose, or any combination thereof.

In certain embodiments, the formulation for injection or infusion includes an aqueous solution; a hydrophobic active agent dispersed to form a colloidal nano-dispersion of particles; and at least one of a dispersion stabilizing agent and an opsonization reducer. The colloidal nano-dispersion of the active agent is dispersed into nano-dispersion particles having sizes of less than 200-nm. In some embodiments the dispersion stabilizing agent is selected from natural or semisynthetic phospholipids. For example, suitable stabilizing agents include polyethoxylated (a/k/a pegylated) castor oil (Cremophor® EL), polyethoxylated hydrogenated castor oil (Cremophor® RH 40), Tocopherol polyethylene glycol succinate (Pegylated vitamin E, Vitamin E TPGS), Sorbitan fatty acid esters (Spans®), Bile acids and bile-acid salts or Dimyristoylphosphatidyl choline (DMPC). In some embodiments the stabilizing agent is DMPC.

In certain embodiments the formulation is suitable for parenteral administration, including intravenous, intraperitoneal, orthotopical, intracranial, intramuscular, subcutaneous, intramedullary injections, as well as intrathecal, direct intraventricular, intranasal, or intraocular injections. In certain embodiments, the formulation contains CoQ10, dimyristoyl-phophatidylcholine, and poloxamer 188 in a ratio of 4:3:1.5 respectively that is designed to stabilize the nanosuspension of the particles. In some embodiments, the formulation includes a phosphate buffer saline solution which contains sodium phosphate dibasic, potassium phosphate monobasic, potassium chloride, sodium chloride and water for injection. In certain embodiments, the 4% sterile aqueous colloidal dispersion containing CoQ10 in a nanosuspension is diluted in the phosphate buffered saline solution provided, e.g., 1:1, 1:2, 1:3, 1:4. 1:5, 1:6, 1:7, 1:8. 1:9, 1:10, 1:11, 1:12, 1:13, 1:14. 1:15, 1:16, 1:17, 1:18. 1:19, 1:20, or other appropriate ratio bracketed by any two of the values.

In some embodiments, the formulation is a topical formulation. Topical formulations of CoQ10 compounds are provided, for example in WO2010/132507 (PCT Appln. No. PCT/US2010/034453), WO2008116135 (PCT Appln. No. PCT/US2008/116135), and WO2005/069916 (PCT Appln. PC/US2005/001581), the entire contents of each of which are expressly incorporated herein by reference.

Formulations suitable for topical administration include liquid or semi-liquid preparations suitable for penetration through the skin, such as liniments, lotions, creams, ointments or pastes, and drops suitable for administration to the eye, ear, or nose. Drops according to the present disclosure may include sterile aqueous or oily solutions or suspensions and may be prepared by dissolving the active ingredient in a suitable aqueous solution of a bactericidal and/or fungicidal agent and/or any other suitable preservative, and in some embodiments including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present disclosure include those suitable for application to the skin or eye. An eye lotion may include a sterile aqueous solution optionally containing a bactericide and may be prepared by methods similar to those for the preparation of drops. Lotions or liniments for application to the skin may also include an agent to hasten drying and to cool the skin, such as an alcohol, and/or a moisturizer such as glycerol or an oil such as castor oil or arachis oil.

Creams, ointments or pastes useful in the methods of the invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may include hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active such as sorbitan esters or polyoxyethylene derivatives thereof. Suspending agents such as natural gums, cellulose derivatives or inorganic materials such as silicaceous silicas, and other ingredients such as lanolin, may also be included.

In some embodiments, the remaining component of a topical delivery vehicle may be water or a water phase, in embodiments purified, e.g. deionized, water, glycerine, propylene glycol, ethoxydiglycol, phenoxyethanol, and cross linked acrylic acid polymers. Such delivery vehicle compositions may contain water or a water phase in an amount of from about 50 to about 95 percent, based on the total weight of the composition. The specific amount of water present is not critical, however, being adjustable to obtain the desired viscosity (usually about 50 cps to about 10,000 cps) and/or concentration of the other components. The topical delivery vehicle may have a viscosity of at least about 30 centipoises.

Topical formulations can also include an oil phase including, for example, oil phase which, in turn, may include emollients, fatty alcohols, emulsifiers, combinations thereof, and the like. For example, an oil phase could include emollients such as C12-15 alkyl benzoates (commercially available as FINSOLV™ TN from Finetex Inc. (Edison, N.J.)), capric-caprylic triglycerides (commercially available from Huls as MIGLYOL™ 812), and the like. Other suitable emollients which may be utilized include vegetable derived oils (corn oil, safflower oil, olive oil, macadamian nut oil, etc.); various synthetic esters, including caprates, linoleates, dilinoleates, isostearates, fumarates, sebacates, lactates, citrates, stearates, palmitates, and the like; synthetic medium chain triglycerides, silicone oils or polymers; fatty alcohols such as cetyl alcohol, stearyl alcohol, cetearyl alcohol, lauryl alcohol, combinations thereof, and the like; and emulsifiers including glyceryl stearate, PEG-100 stearate, Glyceryl Stearate, Glyceryl Stearate SE, neutralized or partially neutralized fatty acids, including stearic, palmitic, oleic, and the like; vegetable oil extracts containing fatty acids, Ceteareth®-20, Ceteth®-20, PEG-150 Stearate, PEG-8 Laurate, PEG-8 Oleate, PEG-8 Stearate, PEG-20 Stearate, PEG-40 Stearate, PEG-150 Distearate, PEG-8 Distearate, combinations thereof, and the like; or other non-polar cosmetic or pharmaceutically acceptable materials used for skin emolliency within the purview of those skilled in the art, combinations thereof, and the like.

Topical formulations can also include a liposomal concentrate including, for example, a phospholipid such as lecithin, lysolecithin, phosphatidylcholine, phosphatidylethanolamine, phosphatidylinositol, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, and combinations thereof, at least one lipophilic bioactive agent, and at least one solubilizer. The liposomal concentrate may be in combination with at least one pharmaceutically acceptable carrier possessing at least one permeation enhancer in an amount from about 0.5% by weight to about 20% by weight of the composition. The phospholipid may present in the composition in an amount from about 2% to about 20% by weight of the composition and the bioactive agent may be present in an amount from about 0.5% to about 20% by weight of the composition.

Transdermal skin penetration enhancers can also be used to facilitate delivery of CoQ10. Illustrative are sulfoxides such as ethoxydiglycol, 1,3-butylene glycol, isopentyl diol, 1,2-pentane diol, propylene glycol, 2-methyl propan-2-ol, propan-2-ol, ethyl-2-hydroxypropanoate, hexan-2,5-diol, di(2-hydroxypropyl)ether, pentan-2,4-diol, acetone, polyoxyethylene(2)methyl ether, 2-hydroxypropionic acid, 2-hydroxyoctanoic acid, propan-1-ol, 1,4 dioxane, tetrahydrofuran, butan-1,4-diol, propylene glycol dipelargonate, polyoxypropylene 15 stearyl ether, octyl alcohol, polyoxyethylene ester of oleyl alcohol, oleyl alcohol, lauryl alcohol, dioctyl adipate, dicapryl adipate, diisopropyl adipate, diisopropyl sebacate, dibutyl sebacate, diethyl sebacate, dimethyl sebacate, dioctyl sebacate, dibuyl suberate, dioctyl azelate, dibenzyl sebacate, dibutyl phthalate, dibutyl azelate, ethyl myristate, dimethyl azelate, butyl myristate, dibutyl succinate, didecyl phthalate, decyl oleate, ethyl caproate, ethyl salicylate, isopropyl palmitate, ethyl laurate, 2-ethyl-hexyl pelargonate, isopropyl isostearate, butyl laurate, benzyl benzoate, butyl benzoate, hexyl laurate, ethyl caprate, ethyl caprylate, butyl stearate, benzyl salicylate, 2-hyroxyoctanoic acid, dimethyl sulphoxide, methyl sufonyl methane, n,n-dimethyl acetamide, n,n-dimethyl formamide, 2-pyrrolidone, 1-methyl-2-pyrrolidone, 5-methyl-2-pyrrolidone, 1,5-dimethyl-2-pyrrolidone, 1-ethyl-2-pyrrolidone, phosphine oxides, sugar esters, tetrahydrofurfural alcohol, urea, diethyl-m-toluamide, 1-dodecylazacyloheptan-2-one, and combinations thereof.

Solubilizers, particularly for topical administration can include, but are not limited to, polyoxyalkylene dextrans, fatty acid esters of saccharose, fatty alcohol ethers of oligoglucosides, fatty acid esters of glycerol, fatty acid esters of polyoxyethylenes, polyethoxylated fatty acid esters of sorbitan, fatty acid esters of poly(ethylene oxide), fatty alcohol ethers of poly(ethylene oxide), alkylphenol ethers of poly(ethylene oxide), polyoxyethylene-polyoxypropylene block copolymers, ethoxylated oils, and combinations thereof.

Topical formulations can include emollients, including, but not limited to, C12-15 alkyl benzoates, capric-caprylic triglycerides, vegetable derived oils, caprates, linoleates, dilinoleates, isostearates, fumarates, sebacates, lactates, citrates, stearates, palmitates, synthetic medium chain triglycerides, silicone oils, polymers and combinations thereof; the fatty alcohol is selected from the group consisting of cetyl alcohol, stearyl alcohol, cetearyl alcohol, lauryl alcohol and combinations thereof; and the emulsifier is selected from the group consisting of glyceryl stearate, polyethylene glycol 100 stearate, neutralized fatty acids, partially neutralized fatty acids, polyethylene glycol 150 stearate, polyethylene glycol 8 laurate, polyethylene glycol oleate, polyethylene glycol 8 stearate, polyethylene glycol 20 stearate, polyethylene glycol 40 stearate, polyethylene glycol 150 distearate, polyethylene glycol 8 distearate, and combinations thereof.

Topical formulations can include a neutralization phase comprising one or more of water, amines, sodium lactate, and lactic acid. The water phase can further optionally include one or more of water phase comprises the permeation enhancer optionally in combination with a viscosity modifier selected from the group consisting of cross linked acrylic acid polymers, pullulan, mannan, scleroglucans, polyvinylpyrrolidone, polyvinyl alcohol, guar gum, hydroxypropyl guar gum, xanthan gum, acacia gum, arabia gum, tragacanth, galactan, carob gum, karaya gum, locust bean gum, carrageenin, pectin, amylopectin, agar, quince seed, rice starch, corn starch, potato starch, wheat starch, algae extract, dextran, succinoglucan, carboxymethyl starch, methylhydroxypropyl starch, sodium alginate, alginic acid propylene glycol esters, sodium polyacrylate, polyethylacrylate, polyacrylamide, polyethyleneimine, bentonite, aluminum magnesium silicate, laponite, hectonite, and anhydrous silicic acid.

Topical formulations can also include a pigment such as titanium dioxide. In an embodiment, a topical formulation for use in the methods of the invention includes an oil phase comprising C12-15 alkyl benzoates or capric/caprylic triglyceride, cetyl alcohol, stearyl alcohol, glyceryl stearate, and polyethylene glycol 100 stearate, in an amount of from about 5% to about 20% by weight of the composition; a water phase comprising glycerin, propylene glycol, ethoxydiglycol, phenoxyethanol, water, and a crosslinked acrylic acid polymer, in an amount of from about 60 to about 80% by weight of the composition; a neutralization phase comprising water, triethanolamine, sodium lactate, and lactic acid, in an amount of from about 0.1% to about 15% by weight of the composition; a pigment comprising titanium dioxide in an amount of from about 0.2% to about 2% by weight of the composition; and a liposomal concentrate comprising a polyethoxylated fatty acid ester of sorbitan, coenzyme Q10, a phosphatidylcholine lecithin, phenoxyethanol, propylene glycol, and water, in an amount of from about 0.1% to about 30% by weight of the composition, wherein the propylene glycol and ethoxydiglycol are present in a combined amount of from 3% by weight to about 15% by weight of the composition and the coenzyme Q10 is present in an amount of from about 0.75% by weight to about 10% by weight of the composition. Other formulations for use in the methods of the invention are provided, for example, in WO2008/116135 (PCT Application No. PCT/US08/57786), and in WO2010/132507 (PCT/US2010/034453), the entire contents of each of which are expressly incorporated herein by reference.

In one embodiment, a topical formulation for use in the methods of the invention is a 3% CoQ10 cream as described in US 2011/0027247, the entire contents of which are incorporated by reference herein. In one embodiment, the 3% CoQ10 comprises: (1) a phase A having C12-15 alkyl benzoate or capric/caprylic triglyceride at about 4.0% w/w of the composition, cetyl alcohol at about 2.00% w/w of the composition, stearyl alcohol at about 1.5% w/w, glyceryl stearate and PEG-100 at about 4.5% w/w; (2) a phase B having glycerin at about 2.00% w/w, propylene glycol at about 1.5% w/w, ethoxydiglycol at about 5.0% w/w, phenoxyethanol at about 0.475% w/w, a carbomer dispersion at about 40% w/w, purified water at about 16.7% w/w; (3) a phase C having triethanolamine at about 1.3% w/w, lactic acid at about 0.5% w/w, sodium lactate solution at about 2.0% w/w, water at about 2.5% w/w; (4) a phase D having titanium dioxide at about 1.0% w/w; and (5) a phase E having CoQ10 21% concentrate at about 15.0% w/w.

A CoQ10 21% concentrate composition (phase E in above 3% cream) can be prepared by combining phases A and B as described below. Phase A includes Ubidecarenone USP (CoQ10) at 21% w/w and polysorbate 80 NF at 25% w/w. Phase B includes propylene glycol USP at 10.00% w/w, phenoxyethanol NF at 0.50% w/w, lecithin NF (PHOSPHOLIPON 85G) at 8.00% w/w and purified water USP at 35.50% w/w. All weight percentages are relative to the weight of the entire CoQ10 21% concentrate composition. The percentages and further details are listed in the following table.

TABLE 1

| Phase | Trade Name | INCI Name | Percent |
|---|---|---|---|
| A | RITABATE 80 | POLYSORBATE 80 | 25.000 |
| A | UBIDECARENONE | UBIQUINONE | 21.000 |
| B | PURIFIED WATER | WATER | 35.500 |
| B | PROPYLENE GLYCOL | PROPYLENE | 10.000 |
| | | GLYCOL | |
| B | PHENOXYETHANOL | PHENOXYETHANOL | 0.500 |
| B | PHOSPHOLIPON 85G | LECITHIN | 8.000 |
| Totals | | | 100.000 |

The phenoxyethanol and propylene glycol are placed in a suitable container and mixed until clear. The required amount of water is added to a second container (Mix Tank 1). Mix Tank 1 is heated to between 45 and 55° C. while being mixed. The phenoxyethanol/propylene glycol solution is added to the water and mixed until it was clear and uniform. When the contents of the water phase in Mix Tank 1 are within the range of 45 to 55° C., Phospholipon G is added with low to moderate mixing. While avoiding any foaming, the contents of Mix Tank 1 is mixed until the Phospholipon 85G was uniformly dispersed. The polysorbate 89 is added to a suitable container (Mix Tank 2) and heated to between 5° and 60° C. The Ubidecarenone is then added to Mix Tank 2. While maintaining the temperature at between 5° and 60° C. Mix Tank 2 is mixed until all the Ubidecarenone is dissolved. After all the Ubidecarenone has been dissolved, the water phase is slowly transferred to Mix Tank 2. When all materials have been combined, the contents are homogenized until dispersion is smooth and uniform. While being careful not to overheat, the temperature is maintained at between 5° and 60° C. The homogenization is then stopped and the contents of Mix Tank 2 are transferred to a suitable container for storage.

In some embodiments, a formulation for any route of administration for use in the invention may include from about 0.001% to about 20% (w/w) of CoQ10, more preferably between about 0.01% and about 15% and even more preferably between about 0.1% to about 10% (w/w) of CoQ10. In certain embodiments, a formulation for any route of administration for use in the invention may include from about 1% to about 10% (w/w) of CoQ10. In certain embodiments, a formulation for any route of administration for use in the invention may include from about 2% to about 8% (w/w) of CoQ10. In certain embodiments, a formulation for any route of administration for use in the invention may include from about 2% to about 7% (w/w) of CoQ10. In certain embodiments, a formulation for any route of administration for use in the invention may include from about 3% to about 6% (w/w) of CoQ10. In certain embodiments, a formulation for any route of administration for use in the invention may include from about 3% to about 5% (w/w) of CoQ10. In certain embodiments, a formulation for any route of administration for use in the invention may include from about 3.5% to about 4.5% (w/w) of CoQ10. In certain embodiments, a formulation for any route of administration for use in the invention may include from about 3.5% to about 5% (w/w) of CoQ10. In one embodiment a formulation includes about 4% (w/w) of CoQ10. In one embodiment a formulation includes about 8% (w/w) of CoQ10. In various embodiments, the formulation includes about 0.1%, 0.2%, 0.3%, 0.4%, 0.5%, 1%, 2%, 3%, 4%, 5%, 6%, 7%, 8%, 9%, 10%, 11%, 12%, 13%, 14%, 15%, 16%, 17%, 18%, 19% or 20% (w/w) of CoQ10, or any range bracketed by any two values recited. In certain embodiments, the formulations can be prepared as a percent weight to volume rather than a percent weight to weight. Depending on the formulation, the concentration of CoQ10 may be the same, or about the same in the w/w and the w/v percent formulations. CoQ10 can be obtained from Kaneka Q10 as Kaneka Q10 (USP UBIDECARENONE) in powdered form (Pasadena, Texas, USA). CoQ10 used in the methods exemplified herein have the following characteristics: residual solvents meet USP 467 requirement; water content is less than 0.0%, less than 0.05% or less than 0.2%; residue on ignition is 0.0%, less than 0.05%, or less than 0.2% less than; heavy metal content is less than 0.002%, or less than 0.001%; purity of between 98-100% or 99.9%, or 99.5%.

In certain embodiments, the concentration of CoQ10 in the formulation is 1 mg/mL to 150 mg/mL. In one embodiment, the concentration of CoQ10 in the formulation is 5 mg/mL to 125 mg/mL. In one embodiment, the concentration of CoQ10 in the formulation is 10 mg/mL to 100 mg/mL. In one embodiment, the concentration of CoQ10 in the formulation is 20 mg/mL to 90 mg/mL. In one embodiment, the concentration of CoQ10 is 30 mg/mL to 80 mg/mL. In one embodiment, the concentration of CoQ10 is 30 mg/mL to 70 mg/mL. In one embodiment, the concentration of CoQ10 is 30 mg/mL to 60 mg/mL. In one embodiment, the concentration of CoQ10 is 30 mg/mL to 50 mg/mL. In one embodiment, the concentration of CoQ10 is 35 mg/mL to 45 mg/mL. It should be understood that additional ranges having any one of the foregoing values as the upper or lower limits are also intended to be part of this invention, e.g., 10 mg/mL to 50 mg/mL, or 20 mg/mL to 60 mg/mL.

In certain embodiments, the concentration of CoQ10 in the formulation is about 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90 or 95 mg/mL. In one embodiment, the concentration of CoQ10 in the formulation is about 50 mg/mL. In one embodiment, the concentration of CoQ10 in the formulation is about 60 mg/mL. In one embodiment, the concentration of CoQ10 in the formulation is about 30 mg/mL. In a preferred embodiment, the concentration of CoQ10 in the formulation is about 40 mg/mL. It should be understood that ranges having any one of these values as the upper or lower limits are also intended to be part of this invention, e.g. between 37 mg/mL and 47 mg/mL, or between 31 mg/mL and 49 mg/mL.

It is understood that formulations can similarly be prepared containing CoQ10 precursors, metabolites, and related compounds.

### VI. Methods of Treatment

Provided herein are methods of treating an oncological disorder in a subject in need thereof, comprising administering a Coenzyme Q10 molecule (e.g. CoQ10 or ubiquinone) to the subject; and administering at least one immune checkpoint modulator of an immune checkpoint molecule to the subject, such that the oncological disorder is treated.

### Coenzyme Q10 Compositions and Administration

In the methods of the invention, the Coenzyme Q10 molecule (e.g, CoQ10 or ubiquinone) can be administered in the form of a pharmaceutical composition, such as the compositions and formulations described herein. In some embodiments, the CoQ10 administered in combination with the at least one immune checkpoint modulator is formulated for intravenous administration, administration by inhalation, topical administration, or oral administration. In certain embodiments, the CoQ10 formulation is not an oral formulation. Intravenous CoQ10 formulations are disclosed, for example, in WO2011/112900, the entire disclosure of which is incorporated by reference herein in its entirety. Topical CoQ10 formulations are disclosed, for example, in US2011/0027247, the entire disclosure of which is incorporated by reference herein in its entirety. Suitable inhalation CoQ10 formulations are disclosed in US 2012/0321698, and US2011/0142914, the entire disclosures of which are incorporated herein in their entirety.

In some embodiments, a CoQ10 formulation may include from about 0.001% to about 20% (w/w) of CoQ10, more preferably between about 0.01% and about 15% and even more preferably between about 0.1% to about 10% (w/w) of CoQ10, more preferably about 3% to about 5% (w/w) of CoQ10. In one embodiment a formulation includes about 4% (w/w) of CoQ10. In one embodiment a formulation includes about 8% (w/w) of CoQ10. In various embodiments, the formulation includes about 0.5%, 1%, 2%, 3%, 4%, 5%, 6%, 7%, 8%, 9%, 10%, 11%, 12%, 13%, 14%, 15%, 16%, 17%, 18%, 19% or 20% (w/w) of CoQ10. As also noted herein, compositions of the present disclosure may be in a liquid form, capable of introduction into a subject by any means or route of administration within the purview of those skilled in the art.

WO/2009/126764 discloses the treatment of cancer with CoQ10; WO2011/11290 discloses intravenous formulations of CoQ10; US2011/0027247 discloses methods of treating oncological disorders using topically administered CoQ10; WO2009073843 and WO2012174559 disclose formulations of CoQ10 for administration by inhalation; each of these applications is hereby incorporated by reference in its entirety.

In certain embodiments, the Coenzyme Q10 composition is administered by routes of administration including, but not limited to, intravenous, intratumoral, intraperitoneal, combinations thereof, and the like. The CoQ10 and the immune checkpoint modulator need not be delivered by the same route of administration. In certain embodiments, the CoQ10 is not administered orally. In one embodiment, a CoQ10 composition suitable for intravenous (IV) administration can be used in combination therapy with at least one immune checkpoint modulator according to the methods of the invention. In one embodiment, a CoQ10 composition suitable for topical administration can be used in combination therapy with at least one immune checkpoint modulator according to the methods of the invention. In one embodiment, a CoQ10 composition suitable for inhalable administration can be used in combination therapy with at least one immune checkpoint modulator according to the methods of the invention. In one embodiment, a CoQ10 composition suitable for oral administration can be used in combination therapy with at least one immune checkpoint modulator according to the methods of the invention. In the methods of the invention, the Coenzyme Q10 molecule (i.e. CoQ10 or ubiquinone) can be administered by any mode of administration appropriate for the cancer being treated. For example, suitable routes of administration include, but are not limited to, topical, oral, inhalation, intraperitoneal, intravenous or intratumoral administration. In a particular embodiment, the methods of the invention comprise treatment of an oncological disorder by continuous infusion of Coenzyme Q10 in combination therapy with at least one immune checkpoint modulators.

In one embodiment of the combination treatment methods provided herein, the CoQ10 formulation is administered one time per week. In one embodiment, the CoQ10 formulation is administered 2 times per week. In one embodiment, the CoQ10 formulation is administered 3 times per week. In one embodiment, the CoQ10 formulation is administered 4 times per week. In another embodiment, the CoQ10 formulation is administered 5 times per week. In one embodiment, the CoQ10 formulation is administered once per day. In one embodiment, the CoQ10 formulation is administered twice per day. In one embodiment, the CoQ10 formulation is administered three times per day.

In some embodiments, the CoQ10 is formulated for IV administration and the dosage is administered by infusion over about 1 hour, 2 hours, 3 hours, 4 hours or longer. In one embodiment, the CoQ10 is administered by intravenous infusion over about 4 hours. In a particular embodiment, the CoQ10 compositions may be administered by continuous infustion. In one embodiment, the CoQ10 is administered by intravenous infusion (e.g. by continuous infusion) over about 24 hours, 36 hours, 48 hours, 60 hours, 72 hours, 84 hours or 96 hours. In certain embodiments, the CoQ10 formulation is administered by intravenous infusion (e.g. by continuous infusion) over about 6, 8, 10, 12, 14, 16, 18, 20, 22 24, 48, 72, 96, 120, 144, 168, 192, 216, 240, 288, 312, 336, 360, 384, 408, 432, 456 or 480 hours. In certain embodiments, the coenzyme Q10 is administered by intravenous infusion (e.g. continuous infusion) for at least 24 hours, at least 48 hours, at least 72 hours, at least 96 hours, 120 hours, for at least 144 hours, for at least 168 hours, for at least 192 hours, for at least 216 hours, for at least 240 hours, for at least 288 hours, for at least 312 hours, for at least 336 hours, for at least 360 hours, for at least 384 hours, for at least 408 hours, for at least 432 hours, for at least 456 hours, or for at least 480 hours.

In certain embodiments, the CoQ10 is administered in at least one dose per day. In certain embodiments, the CoQ10 is administered in at least two doses per day. In certain embodiments, the CoQ10 is administered in at least three dose per day. In certain embodiments, the CoQ10 is administered in one dose per day. In certain embodiments, the CoQ10 is administered in two doses per day. In certain embodiments, the CoQ10 is administered in three doses per day. Additional suitable treatment regimens for Coenzyme Q10 are provided, for example, in US 2015/0157559, the entire contents of which are expressly incorporated herein by reference.

One skilled in the art would be able, by routine experimentation, to determine what an effective, non-toxic amount of a CoQ10 molecule (e.g. CoQ10 or ubiquinone) would be for the purpose of treating oncological disorders. For example, a therapeutically active amount of CoQ10 may vary according to factors such as the disease stage (e.g., stage I versus stage IV), age, sex, medical complications (e.g., immunosuppressed conditions or diseases) and weight of the subject, and the ability of the CoQ10 to elicit a desired response in the subject. The dosage regimen may be adjusted to provide the optimum therapeutic response. For example, several divided doses may be administered daily or administered by continuous infusion or the dose may be proportionally reduced as indicated by the exigencies of the therapeutic situation. In certain embodiments, Coenzyme Q10 is administered in an amount that would be therapeutically effective if delivered alone, i.e., Coenzyme Q10 is administered and/or acts as a therapeutic anti-cancer agent, and not predominantly as an agent to ameliorate side effects of other chemotherapy or other cancer treatments.

In certain embodiments, Coenzyme Q10 is administered in an amount that would be effective to improve or augment the immune response to the tumor, e.g., by augmenting the therapeutic effect of one or more immunce checkpoint modulators. The dosages provided below may be used for any mode of administration of Coenzyme Q10, including topical administration, administration by inhalation, and intravenous administration (e.g. continuous infusion).

In certain embodiments, the subject is administered a dose of CoQ10 in the range of about 0.5 mg/kg to about 10,000 mg/kg, about 5 mg/kg to about 5,000 mg/kg, about 10 mg/kg to about 3,000 mg/kg. In one embodiment, Coenzyme Q10 is administered in the range of about 10 mg/kg to about 1,400 mg/kg. In one embodiment, Coenzyme Q10 is administered in the range of about 10 mg/kg to about 650 mg/kg. In one embodiment, Coenzyme Q10 is administered in the range of about 10 mg/kg to about 200 mg/kg. In various embodiments, Coenzyme Q10 is administered at a dose of about 2 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 58 mg/kg, 58.6 mg/kg, 60 mg/kg, 65 mg/kg, 70 mg/kg, 75 mg/kg, 78 mg/kg, 80 mg/kg, 85 mg/kg, 90 mg/kg, 95 mg/kg, 100 mg/kg, 104 mg/kg, 110 mg/kg, 120 mg/kg, 130 mg/kg, 140 mg/kg, 150 mg/kg, 160 mg/kg, 170 mg/kg, 180 mg/kg, 190 mg/kg or 200 mg/kg. It should be understood that ranges having any one of these values as the upper or lower limits are also intended to be part of this invention, e.g., about 50 mg/kg to about 200 mg/kg, or about 650 mg/kg to about 1400 mg/kg. In one embodiment the administered dose is at least about 1 mg/kg, at least about 5 mg/kg, at least about 10 mg/kg, at least about 12.5 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 58 mg/kg, at least about 58.6 mg/kg, at least about 60 mg/kg, at least about 75 mg/kg, at least about 78 mg/kg, at least about 100 mg/kg, at least about 104 mg/kg, at least about 125 mg/kg, at least about 150 mg/kg, at least about 175 mg/kg, at least about 200 mg/kg, at least about 300 mg/kg, or at least about 400 mg/kg.

In certain embodiments, the coenzyme Q10 is administered at a dose of about 10 mg/kg/day (24 hours) to about 150 mg/kg/day (24 hours). In certain embodiments, the coenzyme Q10 is administered at a dose selected from the group consisting of about 11.8 mg/kg/day (24 hours), about 12.5 mg/kg/day (24 hours), about 14.4 mg/kg/day (24 hours), about 15.6 mg/kg (24 hours), about 16.5 mg/kg/day (24 hours), about 19 mg/kg/day (24 hours), about 20.4 mg/kg/day (24 hours), about 22 mg/kg/day (24 hours), about 25 mg/kg/day (24 hours), about 27.5 mg/kg/day (24 hours), about 29.3 mg/kg/day (24 hours), about 33 mg/kg/day (24 hours), about 34.2 mg/kg/day (24 hours), about 36.7 mg/kg/day (24 hours), about 41.7 mg/kg/day (24 hours), 42.8 mg/kg/day (24 hours), about 44 mg/kg/day (24 hours), about 45.7 mg/kg/day (24 hours), about 51.9 mg/kg/day (24 hours), about 53.8 mg/kg/day (24 hours), about 55 mg/kg/day (24 hours), about 57 mg/kg/day (24 hours), about 58.7 mg/kg/day (24 hours), about 64.8 mg/kg/day (24 hours), about 66.7 mg/kg/day (24 hours), about 68.5 mg/kg/day (24 hours), about 71.7 mg/kg/day (24 hours), about 73.4 mg/kg/day (24 hours), about 81.5 mg/kg/day (24 hours), about 85.5 mg/kg/day (24 hours), about 91.7 mg/kg/day (24 hours), about 107.5 mg/kg/day (24 hours), about 114.6 mg/kg/day (24 hours), and about 143.3 mg/kg/day (24 hours).

In certain embodiments, the coenzyme Q10 is administered at a dose of about 50 mg/kg/week. In certain embodiments, the coenzyme Q10 is administered at a dose of about 66 mg/kg/week. In certain embodiments, the coenzyme Q10 is administered at a dose of about 88 mg/kg/week. In certain embodiments, the coenzyme Q10 is administered at a dose of about 110 mg/kg/week. In certain embodiments, the coenzyme Q10 is administered at a dose of about 137 mg/kg/week. In certain embodiments, the coenzyme Q10 is administered at a dose of about 171 mg/kg/week. In certain embodiments, the coenzyme Q10 is administered at a dose of about 215 mg/kg/week. In certain embodiments, the coenzyme Q10 is administered at a dose selected from the group consisting of about 38 mg/kg/week, about 50 mg/kg/week, about 66 mg/kg/week, about 76 mg/kg/week, about 88 mg/kg/week, about 100 mg/kg/week, about 110 mg/kg/week, about 132 mg/kg/week, about 137 mg/kg/week, about 171 mg/kg/week, about 176 mg/kg/week, about 215 mg/kg/week, about 220 mg/kg/week, about 274 mg/kg/week, about 342 mg/kg week, and about 430 mg/kg/week.

Dosing ranges for inhaled formulations of CoQ10 may be similar to those used for administration by injection. It is understood that nebulizers or other devices for delivery by inhalation are known in the art and can be used in conjunction with the methods of the invention.

Dosages of topical CoQ10 typically depend on the size of the area to be treated. For example, topically administered CoQ10 can be used for the treatment of skin cancer. CoQ10 is applied topically, typically once or twice per day, to the site of the cancerous lesion in an amount sufficient to cover the lesion. If the subject has many lesions for treatment, the CoQ10 is applied to many sites, increasing the total dose administered to the subject. If the subject has a single lesion, the CoQ10 is applied to the single site.

In some embodiments, the CoQ10 molecule (e.g. CoQ10 or ubiquinone) is administered at a dosage that is different (e.g. lower) than the standard dosages of the immune checkpoint modulator used to treat the oncological disorder under the standard of care for treatment for a particular oncological disorder. In certain embodiments, the administered dosage of the CoQ10 molecule is 5%, 10%, 20%, 30%, 40%, 50%, 60%, 70%, 80%, or 90% lower than the standard dosage of the CoQ10 molecule for a particular oncological disorder. In certain embodiments, the dosage administered of the CoQ10 molecule is 95%, 90%, 85%, 80%, 75%, 70%, 65%, 60%, 55%, 50%, 45%, 40%, 35%, 30%, 25%, 20%, 15%, 10% or 5% of the standard dosage of the CoQ10 molecule for a particular oncological disorder.

### Immune Checkpoint Modulators

Methods are provided for the treatment of oncological disorders by administering a CoQ10 composition in combination with at least one immune checkpoint modulator to a subject. In certain embodiments, the immune checkpoint modulator stimulates the immune response of the subject. For example, in some embodiments, the immune checkpoint modulator stimulates or increases the expression or activity of a stimulatory immune checkpoint (e.g. CD27, CD28, CD40, CD122, OX40, GITR, ICOS, or 4-1BB). In some embodiments, the immune checkpoint modulator inhibits or decreases the expression or activity of an inhibitory immune checkpoint (e.g. A2A4, B7-H3, B7-H4, BTLA, CTLA-4, IDO, KIR, LAG3, PD-1, PD-L1, PD-L2, TIM-3 or VISTA).

In certain embodiments the immune checkpoint modulator targets an immune checkpoint molecule selected from the group consisting of CD27, CD28, CD40, CD122, OX40, GITR, ICOS, 4-1BB, A2A4, B7-H3, B7-H4, BTLA, CTLA-4, IDO, KIR, LAG3, PD-1, PD-L1, PD-L2, TIM-3 and VISTA. In certain embodiments the immune checkpoint modulator targets an immune checkpoint molecule selected from the group consisting of CD27, CD28, CD40, CD122, OX40, GITR, ICOS, 4-1BB, A2A4, B7-H3, B7-H4, BTLA, IDO, KIR, LAG3, PD-1, PD-L1, PD-L2, TIM-3 and VISTA. In a particular embodiment, the immune checkpoint modulator targets an immune checkpoint molecule selected from the group consisting of CTLA-4, PD-L1 and PD-1. In a further particular embodiment the immune checkpoint modulator targets an immune checkpoint molecule selected from PD-L1 and PD-1.

In certain embodiments, the immune checkpoint modulator is not anti-CD40, anti-CD 154, anti-OX40, anti-OX40L, anti-CD28, anti-CD80, anti-CD86, anti-CD70, anti-CD27, anti-HVEM, anti-LIGHT, anti-GITR, anti-GITRL, anti-CTLA-4, soluble OX40L, soluble 4-IBBL, soluble CD154, soluble GITRL, soluble LIGHT, soluble CD70, soluble CD80, soluble CD86, soluble CTLA4-Ig, GVAX®, or a combination thereof. In a particular embodiment, the immune checkpoint modulator is not anti-CTLA-4. In a further particular embodiment, the immune checkpoint molecule that is modulated is not CTLA-4.

In some embodiments, more than one (e.g. 2, 3, 4, 5 or more) immune checkpoint modulator is administered to the subject. Where more than one immune checkpoint modulator is administered, the modulators may each target a stimulatory immune checkpoint molecule, or each target an inhibitory immune checkpoint molecule. In other embodiments, the immune checkpoint modulators include at least one modulator targeting a stimulatory immune checkpoint and at least one immune checkpoint modulator targeting an inhibitory immune checkpoint molecule.

In certain embodiments, the immune checkpoint modulator is a binding protein, for example, an antibody. The term “binding protein”, as used herein, refers to a protein or polypeptide that can specifically bind to a target molecule, e.g. an immune checkpoint molecule. In some embodiments the binding protein is an antibody or antigen binding portion thereof, and the target molecule is an immune checkpoint molecule. In some embodiments the binding protein is a protein or polypeptide that specifically binds to a target molecule (e.g., an immune checkpoint molecule). In some embodiments the binding protein is a ligand. In some embodiments, the binding protein is a fusion protein. In some embodiments, the binding protein is a receptor. Examples of binding proteins that may be used in the methods of the invention include, but are not limited to, a humanized antibody, an antibody Fab fragment, a divalent antibody, an antibody drug conjugate, a scFv, a fusion protein, a bivalent antibody, and a tetravalant antibody.

The term “antibody”, as used herein, refers to any immunoglobulin (Ig) molecule comprised of four polypeptide chains, two heavy (H) chains and two light (L) chains, or any functional fragment, mutant, variant, or derivation thereof. Such mutant, variant, or derivative antibody formats are known in the art. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass. In some embodiments, the antibody is a full-length antibody. In some embodiments, the antibody is a murine antibody. In some embodiments, the antibody is a human antibody. In some embodiments, the antibody is a humanized antibody. In other embodiments, the antibody is a chimeric antibody. Chimeric and humanized antibodies may be prepared by methods well known to those of skill in the art including CDR grafting approaches (see, e.g., U.S. Pat. Nos. 5,843,708; 6,180,370; 5,693,762; 5,585,089; and 5,530,101), chain shuffling strategies (see, e.g., U.S. Pat. No. 5,565,332; Rader et al. (1998) PROC. NAT'L. ACAD. SCI. USA 95: 8910-8915), molecular modeling strategies (U.S. Pat. No. 5,639,641), and the like.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) NATURE 341: 544-546; and WO 90/05144 A1, the contents of which are herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) SCIENCE 242:423-426; and Huston et al. (1988) PROC. NAT'L. ACAD. SCI. USA 85:5879-5883). Such single chain antibodies are also intended to be encompassed within the term “antigen-binding portion” of an antibody. Other forms of single chain antibodies, such as diabodies are also encompassed. Antigen binding portions can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005).

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers found that certain sub-portions within Kabat CDRs adopt nearly identical peptide backbone conformations, despite having great diversity at the level of amino acid sequence (Chothia et al. (1987) J. MOL. BIOL. 196: 901-917, and Chothia et al. (1989) NATURE 342: 877-883). These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan et al. (1995) FASEB J. 9: 133-139, and MacCallum et al. (1996) J. MOL. BIOL. 262(5): 732-45. Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

The term “humanized antibody”, as used herein refers to non-human (e.g., murine) antibodies that are chimeric immunoglobulins, immunoglobulin chains, or fragments thereof (such as Fv, Fab, Fab′, F(ab′)2 or other antigen-binding subsequences of antibodies) which contain minimal sequence derived from a non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al. (1986) NATURE 321: 522-525; Reichmann et al. (1988) NATURE 332: 323-329; and Presta (1992) CURR. OP. STRUCT. BIOL. 2: 593-596, each of which is incorporated by reference herein in its entirety.

The term “immunoconjugate” or “antibody drug conjugate” as used herein refers to the linkage of an antibody or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed in order to form the immunoconjugate. Additionally, the immunoconjugate can be provided in the form of a fusion protein that may be expressed from a polynucleotide encoding the immunoconjugate. As used herein, “fusion protein” refers to proteins created through the joining of two or more genes or gene fragments which originally coded for separate proteins (including peptides and polypeptides). Translation of the fusion gene results in a single protein with functional properties derived from each of the original proteins.

A “bivalent antibody” refers to an antibody or antigen-binding fragment thereof that comprises two antigen-binding sites. The two antigen binding sites may bind to the same antigen, or they may each bind to a different antigen, in which case the antibody or antigen-binding fragment is characterized as “bispecific.” A “tetravalent antibody” refers to an antibody or antigen-binding fragment thereof that comprises four antigen-binding sites. In certain embodiments, the tetravalent antibody is bispecific. In certain embodiments, the tetravalent antibody is multispecific, i.e. binding to more than two different antigens.

Fab (fragment antigen binding) antibody fragments are immunoreactive polypeptides comprising monovalent antigen-binding domains of an antibody composed of a polypeptide consisting of a heavy chain variable region (V_{H}) and heavy chain constant region 1 (C_{HI}) portion and a poly peptide consisting of a light chain variable (V_{L}) and light chain constant (C_{L}) portion, in which the C_{L} and C_{HI} portions are bound together, preferably by a disulfide bond between Cys residues.

In a particular embodiment, the immune checkpoint modulator is a fusion protein, for example, a fusion protein that modulates the activity of an immune checkpoint modulator.

In one embodiment, the immune checkpoint modulator is a therapeutic nucleic acid molecule, for example a nucleic acid that modulates the expression of an immune checkpoint protein or mRNA. Nucleic acid therapeutics are well known in the art. Nucleic acid therapeutics include both single stranded and double stranded (i.e., nucleic acid therapeutics having a complementary region of at least 15 nucleotides in length) nucleic acids that are complementary to a target sequence in a cell. In certain embodiments, the nucleic acid therapeutic is targeted against a nucleic acid sequence encoding an immune checkpoint protein.

Antisense nucleic acid therapeutic agents are single stranded nucleic acid therapeutics, typically about 16 to 30 nucleotides in length, and are complementary to a target nucleic acid sequence in the target cell, either in culture or in an organism.

In another aspect, the agent is a single-stranded antisense RNA molecule. An antisense RNA molecule is complementary to a sequence within the target mRNA. Antisense RNA can inhibit translation in a stoichiometric manner by base pairing to the mRNA and physically obstructing the translation machinery, see Dias, N. et al., (2002) Mol Cancer Ther 1:347-355. The antisense RNA molecule may have about 15-30 nucleotides that are complementary to the target mRNA. Patents directed to antisense nucleic acids, chemical modifications, and therapeutic uses include, for example: U.S. Pat. No. 5,898,031 related to chemically modified RNA-containing therapeutic compounds; U.S. Pat. No. 6,107,094 related methods of using these compounds as therapeutic agents; U.S. Pat. No. 7,432,250 related to methods of treating patients by administering single-stranded chemically modified RNA-like compounds; and U.S. Pat. No. 7,432,249 related to pharmaceutical compositions containing single-stranded chemically modified RNA-like compounds. U.S. Pat. No. 7,629,321 is related to methods of cleaving target mRNA using a single-stranded oligonucleotide having a plurality of RNA nucleosides and at least one chemical modification. The entire contents of each of the patents listed in this paragraph are incorporated herein by reference.

Nucleic acid therapeutic agents for use in the methods of the invention also include double stranded nucleic acid therapeutics. An “RNAi agent,” “double stranded RNAi agent,” double-stranded RNA (dsRNA) molecule, also referred to as “dsRNA agent,” “dsRNA”, “siRNA”, “iRNA agent,” as used interchangeably herein, refers to a complex of ribonucleic acid molecules, having a duplex structure comprising two anti-parallel and substantially complementary, as defined below, nucleic acid strands. As used herein, an RNAi agent can also include dsiRNA (see, e.g., US Patent publication 20070104688, incorporated herein by reference). In general, the majority of nucleotides of each strand are ribonucleotides, but as described herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides. Such modifications may include all types of modifications disclosed herein or known in the art. Any such modifications, as used in a siRNA type molecule, are encompassed by “RNAi agent” for the purposes of this specification and claims. The RNAi agents that are used in the methods of the invention include agents with chemical modifications as disclosed, for example, in WO/2012/037254, and WO 2009/073809, the entire contents of each of which are incorporated herein by reference.

Immune checkpoint modulators may be administered at appropriate dosages to treat the oncological disorder, for example, by using standard dosages. One skilled in the art would be able, by routine experimentation, to determine what an effective, non-toxic amount of an immune checkpoint modulator would be for the purpose of treating oncological disorders. Standard dosages of immune checkpoint modulators are known to a person skilled in the art and may be obtained, for example, from the product insert provided by the manufacturer of the immune checkpoint modulator. Examples of standard dosages of immune checkpoint modulators are provided in Table 2 below. In other embodiments, the immune checkpoint modulator is administered at a dosage that is different (e.g. lower) than the standard dosages of the immune checkpoint modulator used to treat the oncological disorder under the standard of care for treatment for a particular oncological disorder.

TABLE 2
Exemplary Standard Dosages of Immune Checkpoint Modulators

| | Immune | |
|---|---|---|
| | Check- | |
| Immune | point | |
| Checkpoint | Molecule | |
| Modulator | Targeted | Exemplary Standard Dosage |
| Ipilimumab | CTLA-4 | 3 mg/kg administered intravenously over 90 |
| (Yervoy ™) | | minutes every 3 weeks for a total of 4 doses |
| Pembrolizumab | PD-1 | 2 mg/kg administered as an intravenous |
| (Keytruda ™) | | infusion over 30 minutes every 3 weeks until |
| | | disease progression or unacceptable toxicity |
| Atezolizumab | PD-L1 | 1200 mg administered as an intravenous |
| (Tecentriq ™) | | infusion over 60 minutes every 3 weeks |

In certain embodiments, the administered dosage of the immune checkpoint modulator is 5%, 10%, 20%, 30%, 40%, 50%, 60%, 70%, 80%, or 90% lower than the standard dosage of the immune checkpoint modulator for a particular oncological disorder. In certain embodiments, the dosage administered of the immune checkpoint modulator is 95%, 90%, 85%, 80%, 75%, 70%, 65%, 60%, 55%, 50%, 45%, 40%, 35%, 30%, 25%, 20%, 15%, 10% or 5% of the standard dosage of the immune checkpoint modulator for a particular oncological disorder. In one embodiment, where a combination of immune checkpoint modulators are administered, at least one of the immune checkpoint modulators is administered at a dose that is lower than the standard dosage of the immune checkpoint modulator for a particular oncological disorder. In one embodiment, where a combination of immune checkpoint modulators are administered, at least two of the immune checkpoint modulators are administered at a dose that is lower than the standard dosage of the immune checkpoint modulators for a particular oncological disorder. In one embodiment, where a combination of immune checkpoint modulators are administered, at least three of the immune checkpoint modulators are administered at a dose that is lower than the standard dosage of the immune checkpoint modulators for a particular oncological disorder. In one embodiment, where a combination of immune checkpoint modulators are administered, all of the immune checkpoint modulators are administered at a dose that is lower than the standard dosage of the immune checkpoint modulators for a particular oncological disorder. In some embodiments, the immune checkpoint modulator is administered at a dose that is lower than the standard dosage of the immune checkpoint modulator, and the CoQ10 molecule (e.g., Coenzyme Q10) is administered at a dose that is lower than the standard dosage of the CoQ10 molecule.

### Co-Administration of Coenzyme Q10 and Immune Checkpoint Modulators

As used herein, the term “co-administering” or “co-administration” refers to administration of CoQ10 prior to, concurrently or substantially concurrently with, subsequently to, or intermittently with the administration of the immune checkpoint modulator. In certain embodiments, CoQ10 is administered prior to administration of the immune checkpoint modulator. In certain embodiments, CoQ10 is administered prior to and concurrently with the immune checkpoint modulator. In certain embodiments, CoQ10 is administered prior to but not concurrently with the immune checkpoint modulator, i.e., CoQ10 administration is discontinued prior to initiation of treatment with or administration of an immune checkpoint modulator. In certain embodiments, CoQ10 is administered concurrently with the immune checkpoint modulator. In certain embodiments, CoQ10 is administered after administration of the immune checkpoint modulator. In certain embodiments, CoQ10 is administered concurrently with and after administration of the immune checkpoint modulator. In certain embodiments, CoQ10 is administered after administration of the immune checkpoint modulator but not concurrently with the immune checkpoint modulator, i.e. administration of the immune checkpoint modulator is discontinued before initiating administration of CoQ10.

CoQ10 and/or pharmaceutical formulations thereof and the immune checkpoint modulator can act additively or, more preferably, synergistically. In one embodiment, the CoQ10 and immune checkpoint modulator act synergistically. In some embodiments the synergistic effects are in the treatment of the oncological disorder. For example, in one embodiment, the combination of CoQ10 and the immune checkpoint modulator improves the durability, i.e. extends the duration, of the immune response against the cancer that is targeted by the immune checkpoint modulator. In other embodiments the synergistic effects are in modulation of the toxicity associated with the immune checkpoint modulator. In one embodiment, the CoQ10 and the immune checkpoint modulator act additively.

The combination therapies of the present invention may be utilized for the treatment of oncological disorders. In some embodiments, the combination therapy of CoQ10 and the immune checkpoint modulator inhibits tumor cell growth. Accordingly, the invention further provides methods of inhibiting tumor cell growth in a subject, comprising administering a CoQ10 molecule and at least one immune checkpoint modulator to the subject, such that tumor cell growth is inhibited. In certain embodiments, treating cancer comprises extending survival or extending time to tumor progression as compared to control, e.g., a population control. In certain embodiments, the subject is a human subject. In preferred embodiments, the subject is identified as having a tumor prior to administration of the first dose of CoQ10 or the first dose of the immune checkpoint modulator. In certain embodiments, the subject has a tumor at the time of the first administration of CoQ10 or at the time of first administration of the immune checkpoint modulator.

The immune checkpoint modulators are administered at a time relative to administration of the CoQ10 such that the desired therapeutic effect, e.g. a therapeutic or synergistic effect, is achieved. For example, in certain embodiments a sufficient amount of time following administration of CoQ10 may be desirable to effectively augment the efficacy of the immune checkpoint modulator relative to the efficacy of the immune checkpoint modulator alone, or to improve the durability of the effect. In certain embodiments, administration of CoQ10 is initiated at least 8 hours, at least 12 hours, at least 18 hours, at least 24 hours, at least 36 hours, at least 48 hours, at least 3 days, at least 4 days, at least 5 days, at least 6 days, at least 1 week, at least 2 weeks, at least 3 weeks, at least 4 weeks, at least 5 weeks, at least 6 weeks, at least 7 weeks, or at least 8 weeks prior to administration of the first dose of an immune checkpoint modulator. In particular embodiments of the methods of the invention, administration of the at least one immune checkpoint modulator may be initiated at least 24 hours after administration of CoQ10 is initiated, one or more weeks after administration of CoQ10 is initiated, two or more weeks after administration of CoQ10 is initiated, three or more weeks after administration of CoQ10 is initiated, four or more weeks after administration of CoQ10 is initiated, five or more weeks after administration of CoQ10 is initiated, six or more weeks after administration of CoQ10 is initiated, seven or more weeks after administration of CoQ10 is initiated, or eight or more weeks after administration of CoQ10 is initiated. In some embodiments, administration of the at least one immune checkpoint modulator is initiated at least 24 hours after administration of CoQ10 is initiated. In one embodiments administration of the at least one immune checkpoint modulator is initiated from 24 hours to 4 weeks after administration of CoQ10 is initiated. In one embodiment, administration of the at least one immune checkpoint modulator is initiated from 24 hours to 1 week, from 1 to 2 weeks, from 1 to 3 weeks, or from 2 to 4 weeks after administration of CoQ10 is initiated.

In one embodiment, administration of the at least one immune checkpoint modulator is initiated about 1 week after administration of CoQ10 is initiated. In one embodiment, administration of the at least one immune checkpoint modulator is initiated about 2 weeks after administration of CoQ10 is initiated. In one embodiment, administration of the at least one immune checkpoint modulator is initiated about 3 weeks after administration of CoQ10 is initiated. In one embodiment, administration of the at least one immune checkpoint modulator is initiated about 4 weeks after administration of CoQ10 is initiated. In one embodiment, administration of the at least one immune checkpoint modulator is initiated about 5 weeks after administration of CoQ10 is initiated. In one embodiment, administration of the at least one immune checkpoint modulator is initiated about 6 weeks after administration of CoQ10 is initiated. In one embodiment, administration of the at least one immune checkpoint modulator is initiated about 7 weeks after administration of CoQ10 is initiated. In one embodiment, administration of the at least one immune checkpoint modulator is initiated about 8 weeks after administration of CoQ10 is initiated.

In certain embodiments, a loading dose of CoQ10 is administered prior to administration of the immune checkpoint modulator. In certain embodiments, CoQ10 is administered to achieve a steady state level of CoQ10 prior to administration of the immune checkpoint modulator. Where the combination therapy includes intravenous CoQ10 formulations, the subject is intravenously administered the CoQ10 at as dose such that oncological disorders are treated or prevented. In one embodiment, the subject is intravenously administered the CoQ10 such that response to the immune checkpoint modulator is improved, e.g., relative to treatment with the immune checkpoint modulator alone.

In one embodiment, the administration of CoQ10 is discontinued before initiation of treatment with the immune checkpoint modulator, i.e., treatment with the immune checkpoint modulator excludes treatment with CoQ10. In one embodiment, the administration of CoQ10 is continued or resumed after initiation of treatment with the immune checkpoint modulator such that the CoQ10 and immune checkpoint modulator are concurrently administered, e.g., for at least one cycle.

In certain embodiments, at least 1, 2, 3, 4, or 5 cycles of the combination therapy are administered to the subject. The subject is assessed for response criteria at the end of each cycle. The subject is also monitored throughout each cycle for adverse events (e.g., clotting, anemia, liver and kidney function, etc.) to ensure that the treatment regimen is being sufficiently tolerated.

It should be noted that more than one immune checkpoint modulator e.g., 2, 3, 4, 5, or more immune checkpoint modulators, may be administered in combination with coenzyme Q10. For example, in one embodiment, two immune checkpoint modulators may be administered in combination with coenzyme Q10. In one embodiment, three immune checkpoint modulators may be administered in combination with coenzyme Q10. In one embodiment, four immune checkpoint modulators may be administered in combination with coenzyme Q10. In one embodiment, five immune checkpoint modulators may be administered in combination with coenzyme Q10. In some embodiments, the two or more immune checkpoint modulators target the same immune checkpoint molecule. In some embodiments, the two or more immune checkpoint modulators each target different immune checkpoint molecules.

In general, the combination therapy including a CoQ10 molecule (e.g. CoQ10) and the immune checkpoint modulators described herein may be used to therapeutically treat any neoplasm. In various embodiments the oncological disorder is selected from the group consisting of leukemia, a lymphoma, a melanoma, a carcinoma, and a sarcoma. In a particular embodiment, the combination therapy is used to treat solid tumors. In various embodiments of the invention, the combination therapy is used for treatment or prevention of cancer of the brain, central nervous system, head and neck, prostate, breast, testicular, pancreas, liver, colon, bladder, urethra, gall bladder, kidney, lung, non-small cell lung, melanoma, mesothelioma, uterus, cervix, ovary, sarcoma, bone, stomach, skin, and medulloblastoma. In one embodiment, the combination therapy is used to treat triple-negative breast cancer (TNBC). In one embodiment, the combination therapy may be used to treat a leukemia e.g., that presents, migrates or metastasizes to a particular organ such as, e.g., the lung, the liver or the central nervous system.

However, treatment using combination therapies of the invention is not limited to the foregoing types of cancers. Examples of cancers amenable to treatment with the combination therapies include, but are not limited to, for example, glioma, glioblastoma, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, skin cancer, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer. In one embodiment, a CoQ10 molecule (e.g. CoQ10) may be used in combination with an immune checkpoint modulator to treat or prevent various types of skin cancer (e.g., Squamous cell Carcinoma or Basal Cell Carcinoma), pancreatic cancer, breast cancer, prostate cancer, liver cancer, or bone cancer. In one embodiment, the combination therapy including CoQ10 is used for treatment of a skin oncological disorder including, but not limited to, squamous cell carcinomas (including SCCIS (in situ) and more aggressive squamous cell carcinomas), basal cell carcinomas (including superficial, nodular and infiltrating basal cell carcinomas), melanomas, or actinic keratosis. In one embodiment, the oncological disorder or cancer which can be treated with the combination therapy including CoQ10 is not melanoma. In one embodiment, the oncological disorder is merkel cell carcinoma (MCC). In a particular embodiment, the oncological disorder is glioblastoma.

In certain embodiments, the effect that the combination therapy including CoQ10 may have on cancer cells may depend, in part, on the various states of metabolic and oxidative flux exhibited by the cancer cells. CoQ10 may be utilized to interrupt and/or interfere with the conversion of an oncogenic cell's dependency of glycolysis and increased lactate utility. As it relates to a cancer state, this interference with the glycolytic and oxidative flux of the tumor microenvironment may influence apoptosis and angiogenesis in a manner which reduces the development of a cancer cell. In some embodiments, the interaction of CoQ10 with glycolytic and oxidative flux factors may enhance the ability of CoQ10 to exert its restorative apoptotic effect in cancer.

In one embodiment, administration of CoQ10 and the immune checkpoint modulator as described herein results in one or more of, reducing tumor size, weight or volume, increasing time to progression, inhibiting tumor growth and/or prolonging the survival time of a subject having an oncological disorder. In certain embodiments, administration of CoQ10 and the immune checkpoint modulator reduces tumor size, weight or volume, increases time to progression, inhibits tumor growth and/or prolongs the survival time of the subject by at least 1%, 2%, 3%, 4%, 5%, 10%, 20%, 30%, 40%, 50%, 60%, 70%, 80%, 90%, 100%, 200%, 300%, 400% or 500% relative to a corresponding control subject that is administered CoQ10 alone or the immune checkpoint modulator alone. In certain embodiments, administration of CoQ10 and the immune checkpoint modulator reduces tumor size, weight or volume, increases time to progression, inhibits tumor growth and/or prolongs the survival time of a population of subjects afflicted with an onocological disorder by at least 1%, 2%, 3%, 4%, 5%, 10%, 20%, 30%, 40%, 50%, 60%, 70%, 80%, 90%, 100%, 200%, 300%, 400% or 500% relative to a corresponding population of control subjects afflicted with the oncological disorder that is administered CoQ10 alone or the immune checkpoint modulator alone. In other embodiments, administration of CoQ10 and the immune checkpoint modulator stabilizes the oncological disorder in a subject with a progressive oncological disorder prior to treatment.

In another aspect, the invention provides methods for topical administration of CoQ10, especially in the treatment of skin cancer, in combination with administration of immune checkpoint modulators by any route of administration. Such methods include pre-treatment with CoQ10 prior to first administration of the immune checkpoint modulator.

In certain embodiments, treatment with Coenzyme Q10 (e.g. by continuous infusion) and the at least one immune checkpoint modulator is combined with an additional anti-cancer agent such as the standard of care for treatment of the particular cancer to be treated, for example by administering a standard dosage of one or more chemotherapeutic agents. The standard of care for a particular cancer type can be determined by one of skill in the art based on, for example, the type and severity of the cancer, the age, weight, gender, and/or medical history of the subject, and the success or failure of prior treatments. In certain embodiments of the invention, the standard of care includes any one of or a combination of surgery, radiation, hormone therapy, antibody therapy, therapy with growth factors, cytokines, and chemotherapy. In one embodiment, the additional anti-cancer agent is not a CoQ10 molecule and/or an immune checkpoint modulator.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

### EXAMPLES

### Example 1—Expression of T Cell Surface Proteins in Cancer Patients Treated with Coenzyme Q10

The ability of Coenzyme Q10 to modulate immune function in cancer patients was investigated by characterizing the molecular signature in buffy coats of patients administered Coenzyme Q10 for the treatment of solid tumors. A sterile Coenzyme Q10 (Ubidecarenone, USP) nanosuspension was administered intravenously to patients with solid tumors. Coenzyme Q10 was evaluated both as a monotherapy and in combination with standard chemotherapeutic agents (e.g. gemcitabine, 5-fluorouracil and docetaxel). The Coenzyme Q10 was provided as a 4% coenzyme Q10 nanosuspension formulation as described in WO 2011/112900, the entire contents of which are expressly incorporated herein by reference.

The effect of Coenzyme Q10 treatments on protein expression in immune cells within the buffy coat was evaluated. Furthermore, it was determined whether the proteins identified were known to be on the surface of T cells. To complete this analysis, buffy coat samples were subjected to shot gun global proteomic analysis, and a comprehensive list of proteins were generated for samples obtained from 25 patients being treated for solid tumors with Coenzyme Q10 as described above.

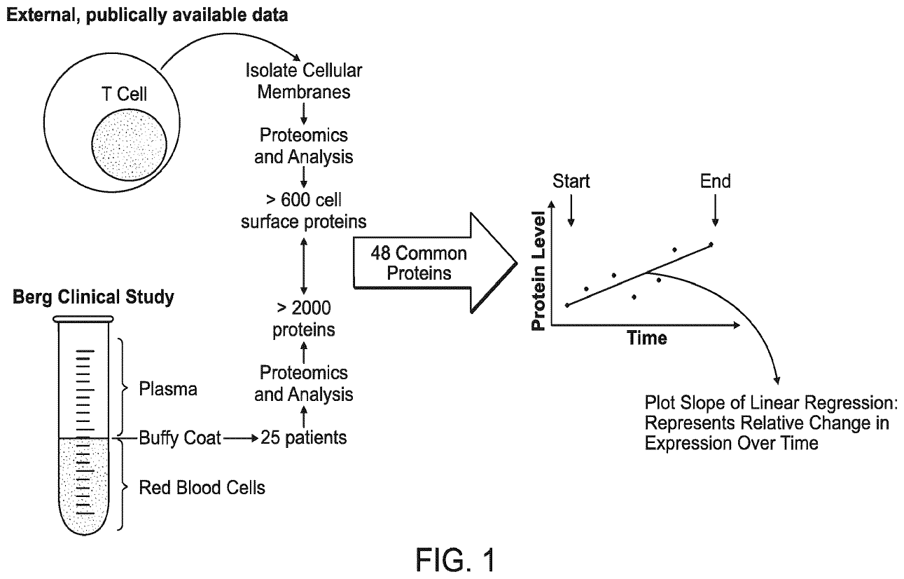

For proteomic analysis, data from two publically available T cell proteomic studies were combined. See Graessel A. et al., 2015, Molecular and Cellular Proteomics 14: 2085-2102; and Loyet K. M. et al., 2005, Proteome Research 4: 400, the entire contents of each of which are expressly incorporated herein by reference. Both studies defined the set of proteins on the surface of naïve and activated T cells to generate a working list of proteins potentially found on the T cell surface. This list (>600 proteins) was compared to the total proteome of the buffy coat (>2000 proteins) from twenty-five patients treated with Coenzyme Q10 as monotherapy. See FIG. 1.

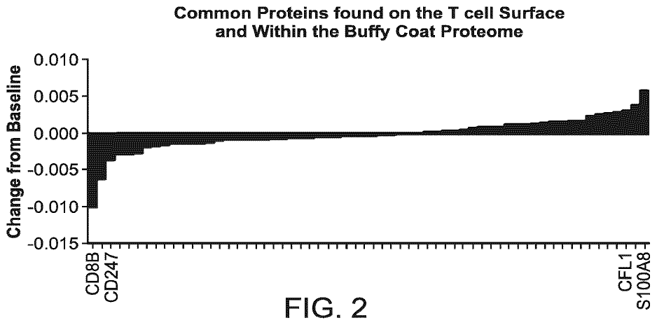

Proteomics analysis revealed a total of 111 proteins that were significantly changed in cells in the buffy coat after treatment with Coenzyme Q10. A total of 62 common proteins shared between T cell surface proteome and the buffy coat proteome were identified. The results are shown in FIG. 2. The relative change in protein levels (on the y-axis) represents the slope of the line derived from the linear regression of the level of each protein at the start of Coenzyme Q10 treatment versus the level at the end of Coenzyme Q10 treatment. FIG. 1 provides a schematic of the plot slope of linear regression representing the change in protein expression over time. In FIG. 2, a positive change from baseline indicates that expression of the protein increased over time, and a negative change from baseline indicates that expression of the protein decreased over time.

Of the 62 T cell proteins differentially expressed in cells in the buffy coat in response to Coenzyme Q10 treatment, four proteins (CD8B, CD247, CFL1, and S100A8) were found to be significantly changed and were also identified on the surface of T cells. Expression of CD8B and CD247 was downregulated by Coenzyme Q10 treatment, and expression of CFL1 and S100A8 was upregulated by Coenzyme Q10 treatment. See FIG. 2. It is important to note that none of the proteins identified in this analysis were observed within the tumor proteome, demonstrating the unique nature of altered expression of these proteins within the cells making up the buffy coat.

CD8 (cluster of differentiation 8) is a transmembrane glycoprotein that serves as a co-receptor for the T cell receptor (TCR). Like the TCR, CD8 binds to a major histocompatibility complex (MHC) molecule, but is specific for the class I MHC protein. There are two isoforms of the protein, alpha (CD8A) and beta (CD8B), each encoded by a different gene. CD8B identifies cytotoxic/suppressor T-cells that interact with MHC class I bearing targets. CD8 is thought to play a role in the process of T-cell mediated killing. See Shiue L., et al., 1988, J Exp Med. 1, 168(6):1993-2005; and Thakral D. et al., 2008, J Immunol 1; 180(11):7431-42, the entire contents of each of which is expressly incorporated by reference herein.

CD247 (Cluster of Differentiation 247) is a T-cell surface glycoprotein that is a subunit of the T cell receptor (TCR) complex. CD247 plays a role in signal transduction upon antigen triggering, and is massively phosphorylated upon antigen recognition. See Christopoulos P. et al., 2015, J Immunol 1; 194(7):3045-53 2015; and Eldor R. et al., 2015, Diabetes Care 38(1):113-8 2015, the entire contents of each of which is expressly incorporated by reference herein.

Cofilin (CFL1) binds to F-actin and exhibits pH-sensitive F-actin depolymerizing activity. CFL1 regulates actin cytoskeleton dynamics and is important for normal progress through mitosis and normal cytokinesis. In addition, CFL1 plays a role in the regulation of cell morphology and cytoskeletal organization, and is required for the up-regulation of atypical chemokine receptor ACKR2 from endosomal compartment to cell membrane, increasing its efficiency in chemokine uptake and degradation. See Mueller C. B., et al., 2015, Oncotarget 28, 6(6):3531-9, the entire contents of which is expressly incorporated by reference herein.

Protein S100-A8 (S100A8) belongs to a class of small calcium-binding proteins and plays a prominent role in regulation of the inflammatory process and immune response and has been shown to modulate CTLA4 expression. See Vandal K. et al., 2003, J Immunology 171:2602; and Basso D. et al., 2013, Oncoimmunology e24441.

Collectively, the differences in upregulation or downregulation of the above T cell surface proteins in response to Coenzyme Q10 treatment is suggestive of T cell activation, proliferation and modulation of activity of lymphocytes. Coenzyme Q10 treatment in cancer patients appears to be associated with the activation of T lymphocyte-mediated innate and adaptive immune response pathways. Thus, combination of Coenzyme Q10 with immunotherapy agents such as immune checkpoint inhibitors has the potential to synergize the activity of these agents in augmenting T cell mediated anti-tumor responses, thereby improving overall durability in patient outcomes.

The proteomic analysis described above indicates that Coenzyme Q10 influences expression of proteins typically expressed on the T cell surface that are associated with T cell activation, proliferation and differentiation. Furthermore, the differential expression of specific markers appears to suggest not only that Coenzyme Q10 influences T cells, but also that Coenzyme Q10 modulates key proteins associated with the function of NK cells in eliciting immune response. These data provide support for combining Coenzyme Q10 with immunotherapy agents to synergize anti-tumor response for durable patient outcomes in various cancers.

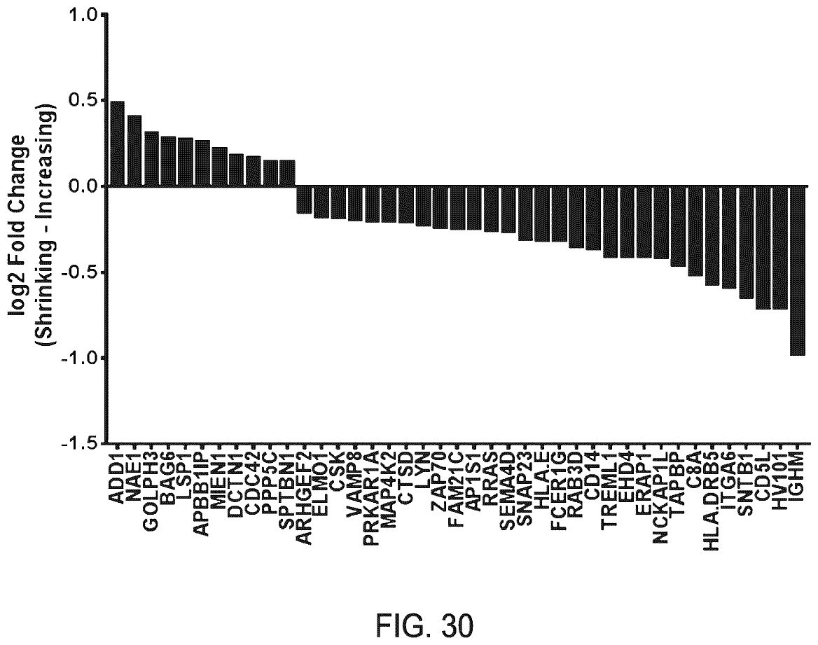

Additionally, proteomic analysis was performed to investigate and identify differentially expressed proteins of leukocytes in growing and shrinking tumors using buffy coat samples of patients administered Coenzyme Q10 for the treatment of solid tumors. The definition of shrinking and growing tumors was based on the identification of tumor slopes corresponding to patient response for the most responsive and least responsive Coenzyme Q10 monotherapy patients during cycle 1. By applying tumor slope classes, any buffy coat proteomics data collected during which the patient's tumor was increasing or decreasing was classified by tumor slope class. Differential expression analysis was performed using linear modeling (limma) (Ritchie et al., 2015, Nucleic Acids Research 43(7): e47) to identify proteins in which their levels were different when measured in growing tumors than in shrinking tumors (increasing or decreasing tumor slopes). Of the 2315 proteins present in the buffy coat data set, 331 were differentially expressed (FDR<0.05) (highest ranked 100). These proteins were run against a dataset focusing on plasma membrane/cell surface associated proteins (Uniprot/TrEMBL dataset: search criteria: human AND reviewed: yes AND organism: “Homo sapiens (Human) [9606 AND cell membrane or Antigen presentation or CD or chemokine receptors). A total of 41 proteins were identified after an additional filtering against a database focusing on cell membrane/cell surface associated proteins (Uniprot/TrEMBL dataset: search criteria: human AND reviewed: yes AND organism:“Homo sapiens (Human) [9606 AND cell membrane or Antigen presentation or CD or chemokine receptors) as shown in FIG. 30.

### Example 2—Effect of Coenzyme Q10 on PDJ, PD-L1 and PD-L2 Expression in Human Cancer Cell Lines

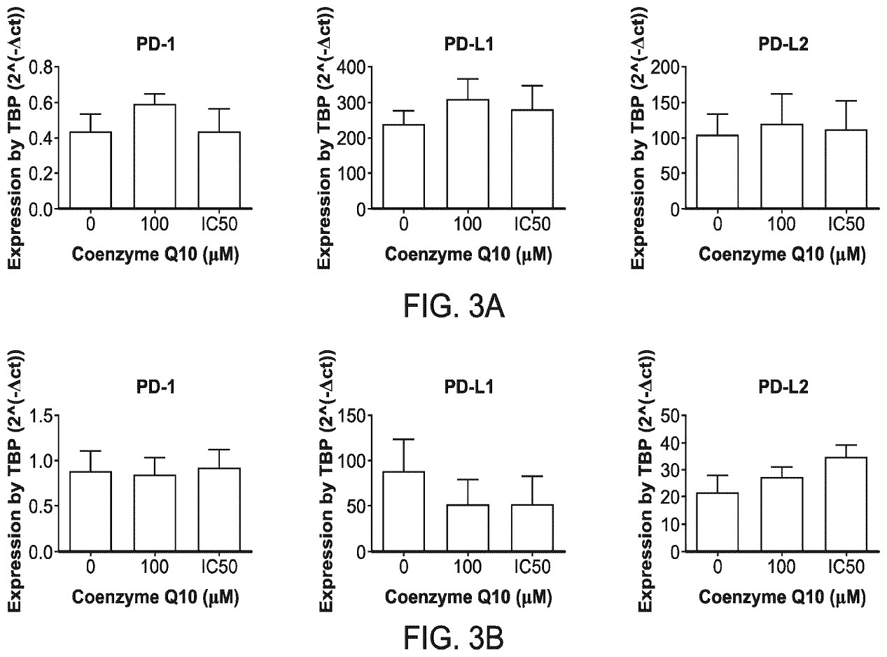

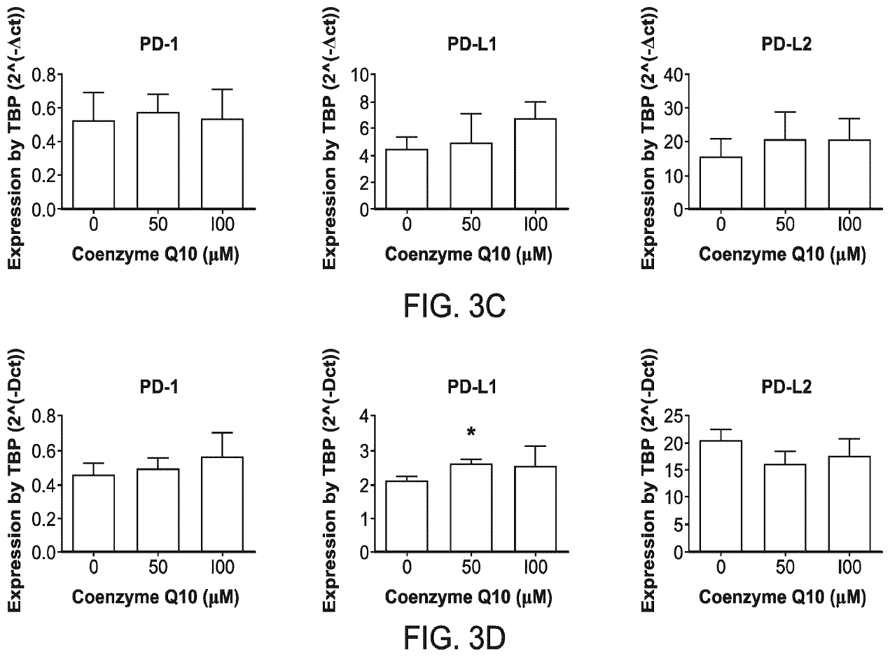

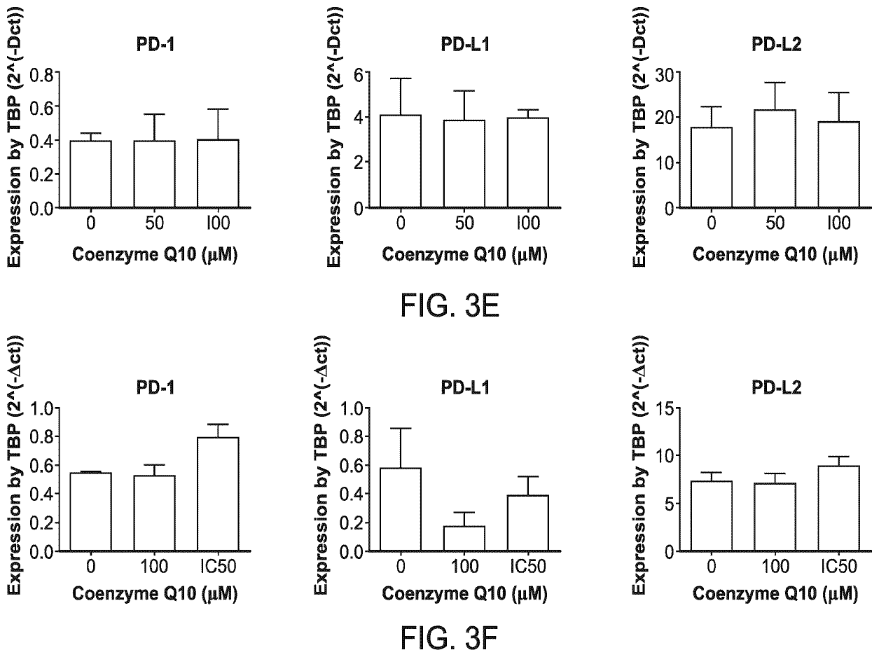

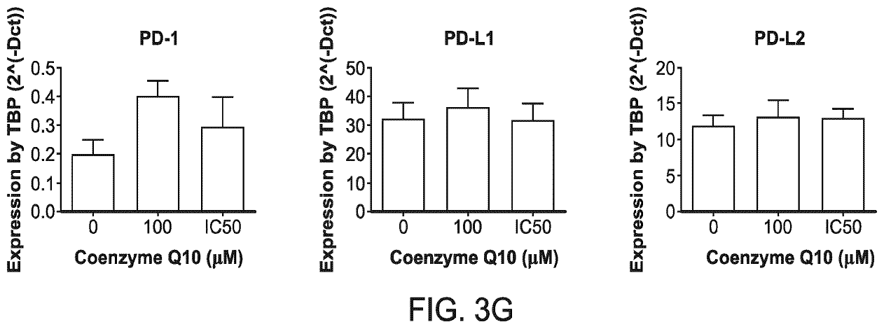

Levels of mRNA expression of the immune checkpoints PD1, PD-L1 and PD-L2 were determined in human breast (MDA-MB231), prostate (LnCAP), ovarian (SKOV-3), colon (HT29), lung (A549), liver (Huh-7), and pancreatic (MIA PaCa-2) cancer cells treated with 50 μM Coenzyme Q10, 100 μM Coenzyme Q10, or the IC₅₀ of Coenzyme Q10 for each cell line. There was a significant increase in PD-L1 mRNA expression in colon cancer cells treated with 50 μM Coenzyme Q10 relative to the untreated cells (*p<0.05; n=3). There were no significant differences among the other treatment groups. See FIGS. 3A-3G. PD1 expression was near the limit of detection of the assay (Ct values of approximately 35), indicating that PD1 is not highly expressed in any of the human cancer cell lines evaluated.

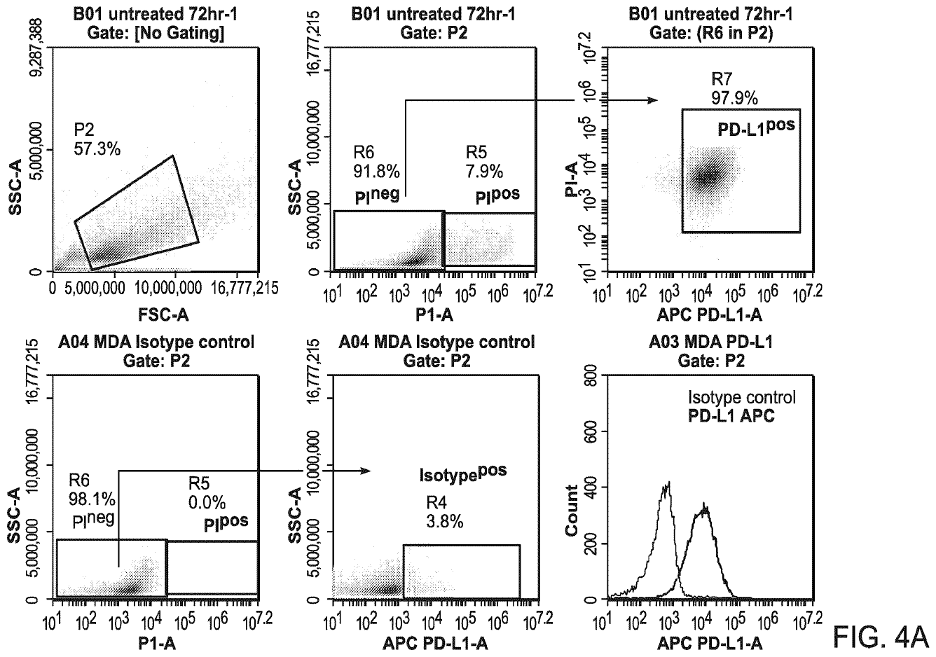

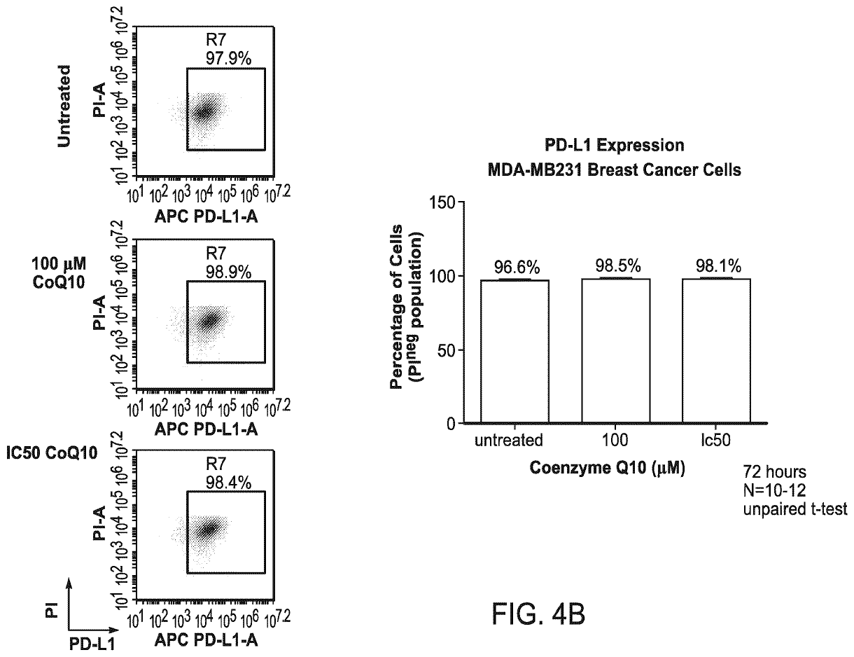

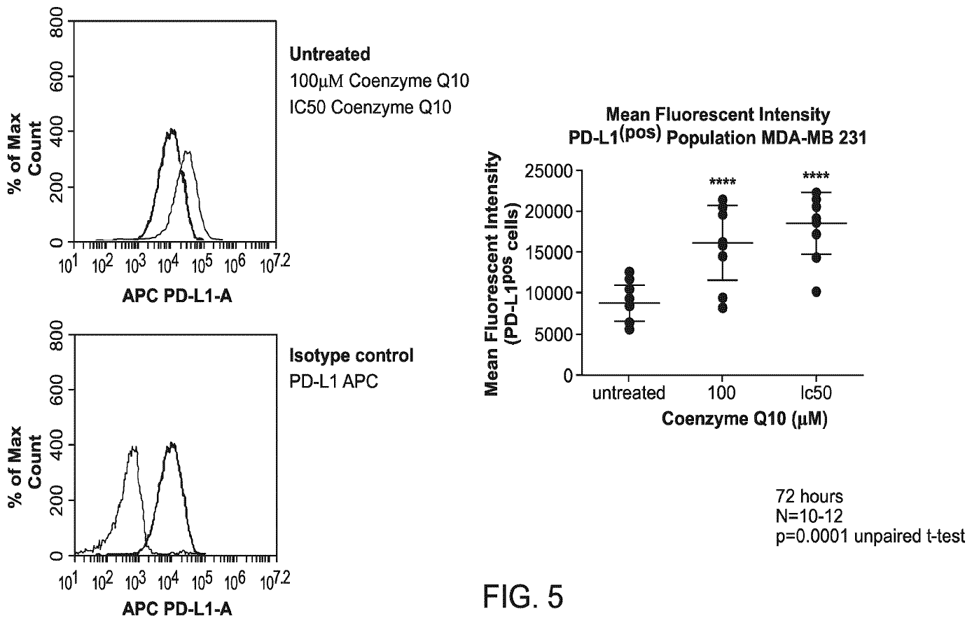

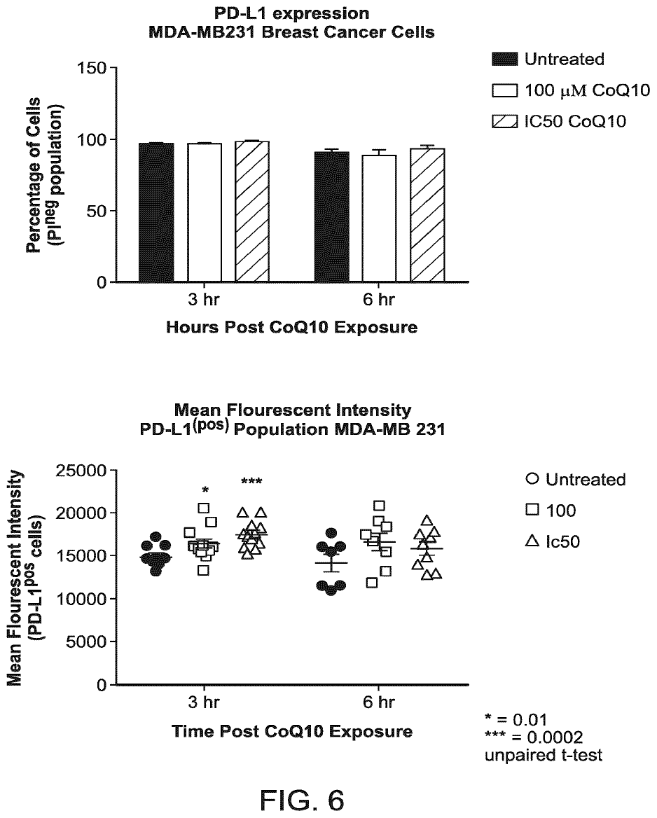

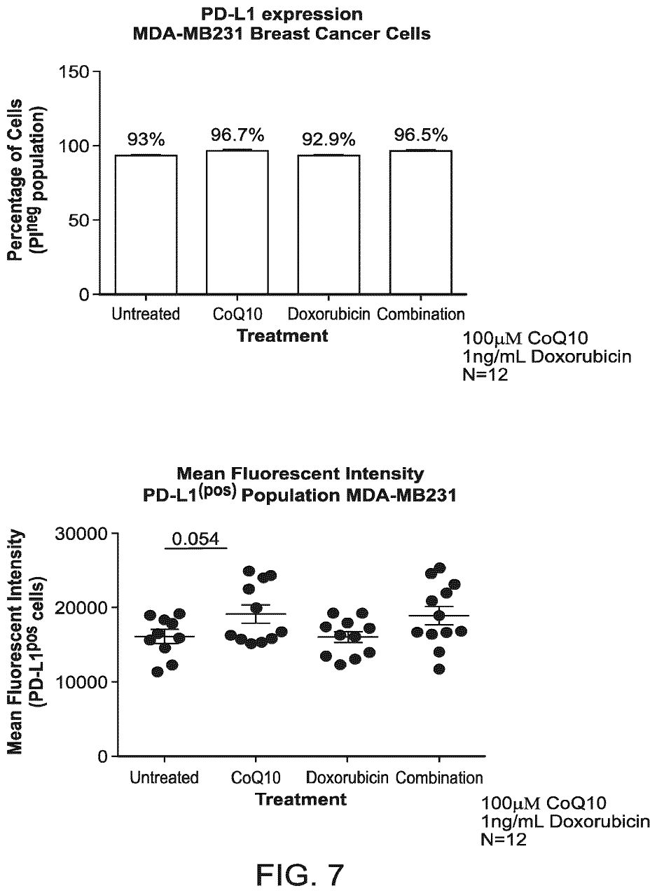

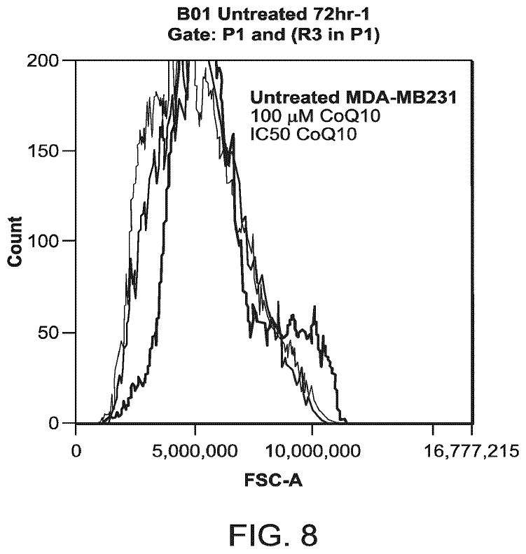

Flow cytometry analysis with a fluorescent probe for PD-L1 was used to determine the expression of PD-L1 protein on the surface of various human cancer cell lines. The cells were treated with 100 μM Coenzyme Q10, or the IC₅₀ of Coenzyme Q10 for each cell line. Coenzyme Q10 did not significantly change the percentage of human breast cancer cells (MDA-MB231) having PD-L1 protein on their surface 72 hours after treatment. See FIGS. 4A and 4B. However, a relatively high percentage of breast cancer cells (96.6%) had PD-L1 protein on their surface even before Coenzyme Q10 treatment, indicating that it may be difficult to detect any further increases caused by Coenzyme Q10. Coenzyme Q10 treatment did significantly increase the amount of PD-L1 protein on the surface of breast cancer cells 72 hours after treatment. See FIG. 5. In addition, Coenzyme Q10 treatment caused transient increases in PD-L1 surface expression in breast cancer cells. For example, Coenzyme Q10 treatment significantly increased the amount of PD-L1 protein on the surface of breast cancer cells 3 hours after treatment, but there was no significant difference between treated and untreated cells 6 hours after treatment. See FIG. 6. Co-treatment with Coenzyme Q10 (100 μM) and doxorubicin (1 ng/mL) did not alter PD-L1 protein levels on the surface of breast cancer cells. See FIG. 7. The increased PD-L1 levels observed in breast cancer cells in response to Coenzyme Q10 treatment were not due to changes in cell populations. As shown in FIG. 8, there were no changes in cell populations between treated and untreated cells.

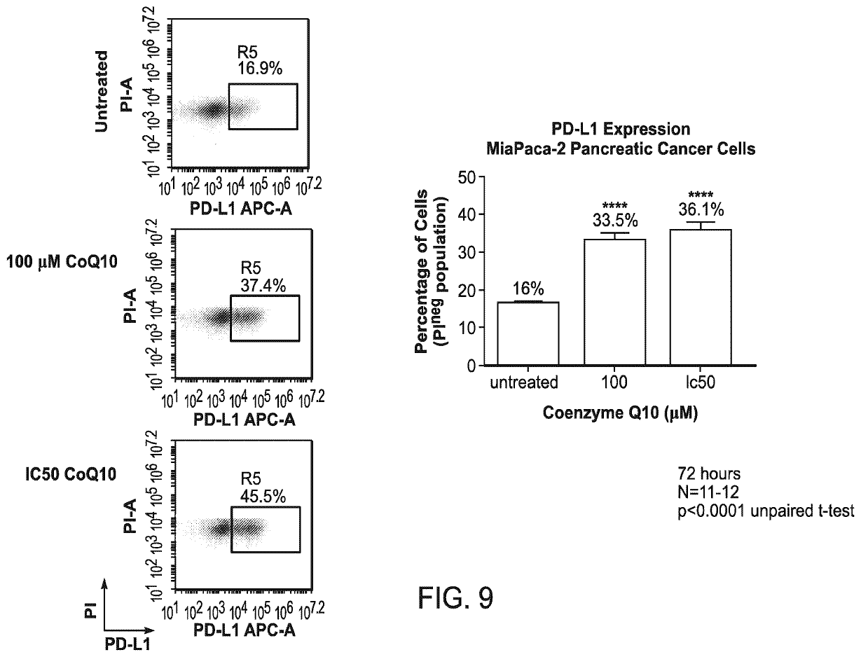

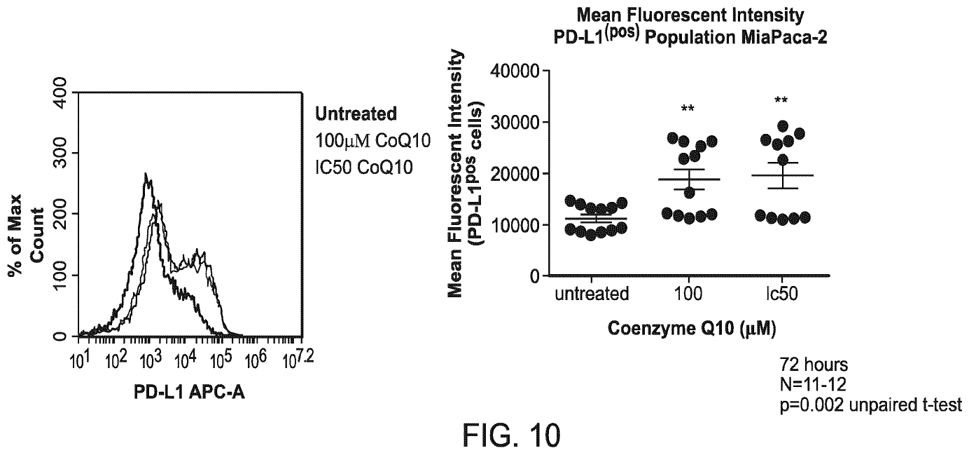

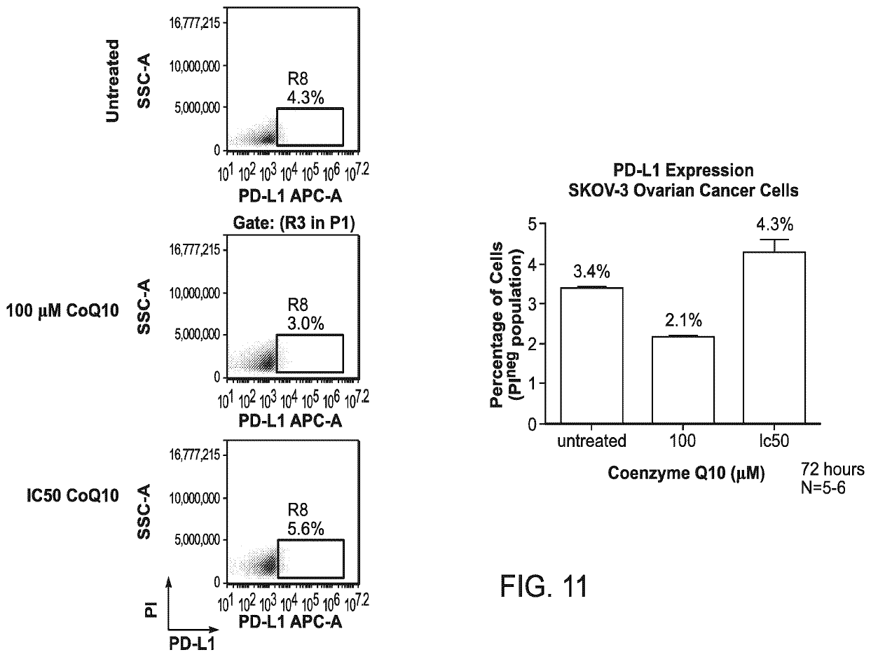

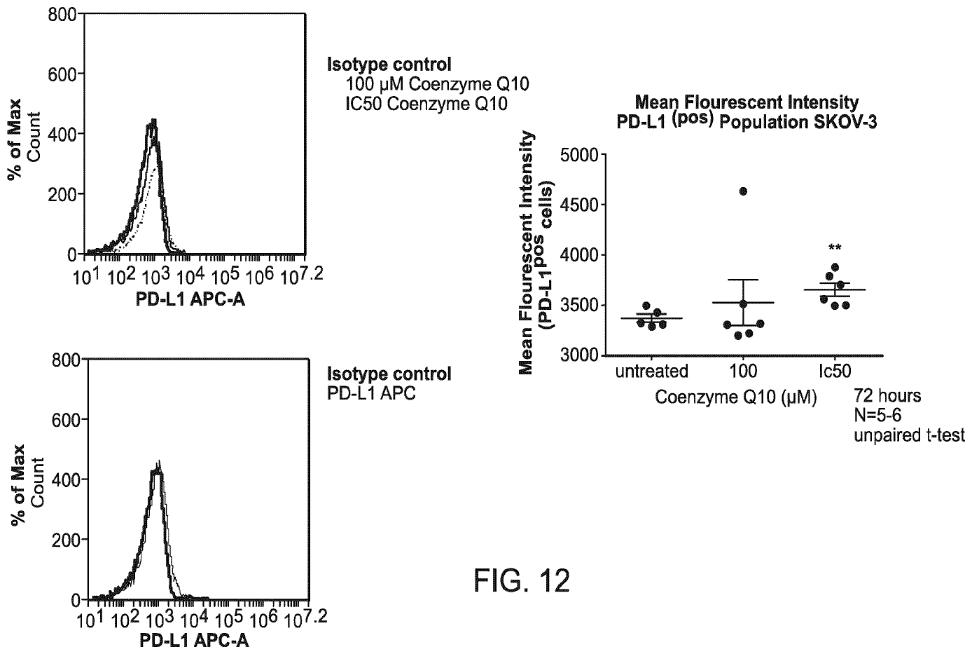

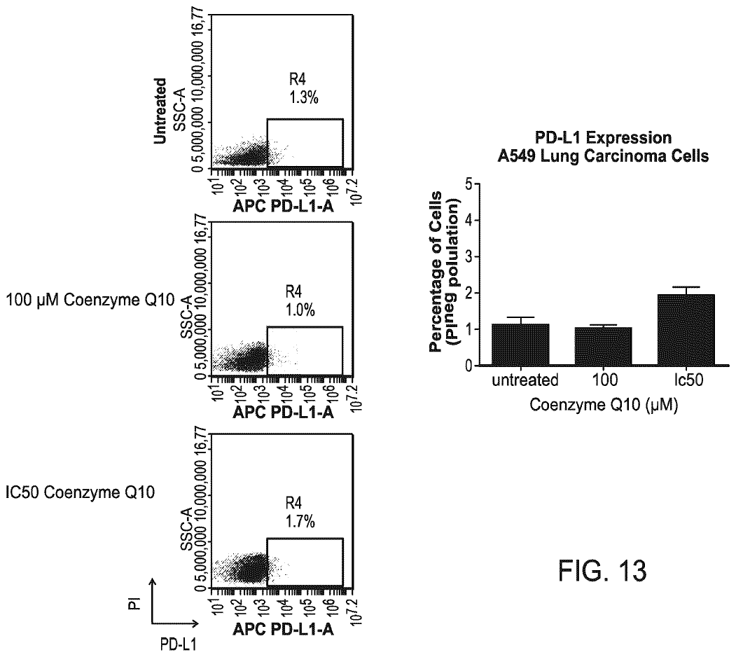

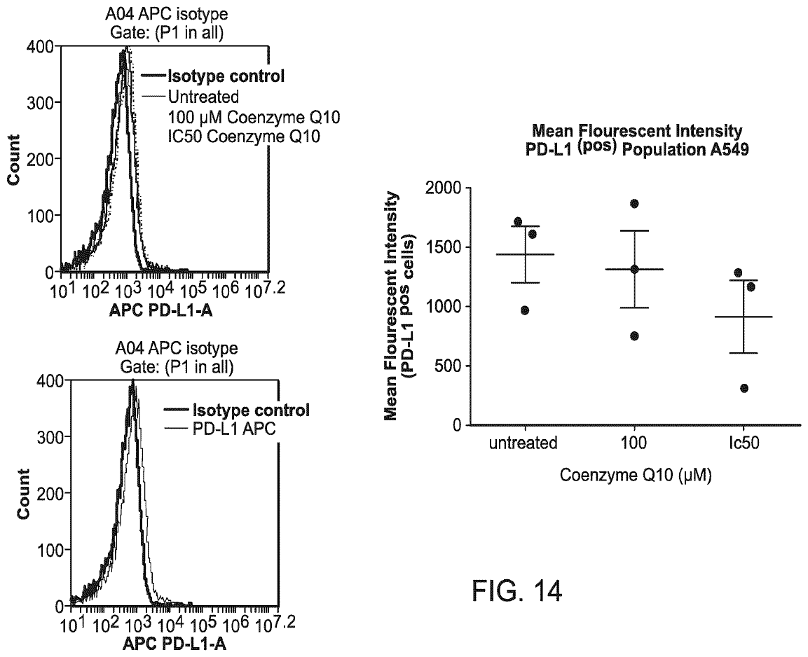

Human pancreatic (MIA PaCa-2), ovarian (SKOV-3), and lung (A549) cancer cell lines treated with Coenzyme Q10 were also analyzed by flow cytometry to determine PD-L1 protein expression on the cancer cell surface. Coenzyme Q10 significantly increased the percentage of pancreatic cancer cells having PD-L1 protein on their surface 72 hours after treatment. See FIG. 9. In addition, Coenzyme Q10 treatment significantly increased the amount of PD-L1 protein on the surface of pancreatic cancer cells 72 hours after treatment. See FIG. 10. Coenzyme Q10 did not significantly change the percentage of ovarian cancer cells having PD-L1 protein on their surface 72 hours after treatment. See FIG. 11. However, Coenzyme Q10 treatment did cause a small but significant increase in the amount of PD-L1 protein on the surface of ovarian cancer cells 72 hours after treatment. See FIG. 12. Coenzyme Q10 did not significantly change the percentage of lung cancer cells having PD-L1 protein on their surface or the amount of PD-L1 on the surface 72 hours after treatment. See FIGS. 13 and 14.

In summary, Coenzyme Q10 treatment increased cell surface levels of PD-L1 in human cancer cells that express moderate to high levels of PD-L1 before treatment, but did not induce cell surface expression of PD-L1 in cells that do not have PD-L1 on their surface before treatment. Co-treatment with Coenzyme Q10 and doxorubicin did not augment the effect of Coenzyme Q10 on cell-surface PD-L1 expression for human breast cancer cells (MDA-MB231).

### Example 3—In Vitro Studies of the Effect of Coenzyme Q10 on Proliferation, Metabolism and PD-L1 Expression in Murine Cancer Cell Lines

Previous in vitro and in vivo studies of the effect of Coenzyme Q10 on cancer have been performed on human cancer cells. In higher mammals, such as humans, which have longer life-spans and slower metabolisms, Coenzyme Q10 is the predominant form of Coenzyme Q (Lass A. et al., 1997, J Biol Chem. 272(31):19199-204.). However, in lower mammals with relatively short life-spans and fast metabolism, the predominant form is Coenzyme Q9. Because Coenzyme Q10 is being evaluated in human patients for treatment of cancer, in vitro experiments will be performed to determine whether Coenzyme Q10 has any effect on the cell metabolism of murine cell lines. The in vitro assays will be focused to determine the EC₅₀ of Coenzyme Q10 in the murine cancer cell lines and to determine Oxygen Consumption Rate (OCR) and Extracellular Acidification Rate (ECAR) on the murine cancer cell lines that will be used for in vivo analysis.

Many tumor cells have developed the ability to express high levels of PD-L1 to suppress immune response against themselves. Before evaluation of anti-PD-L1 antibody in vivo, it is important to identify the level of PD-L1 on the surface of the mouse cancer cell lines that are chosen for the in vivo studies. Additionally, because previous studies have demonstrated that Coenzyme Q10 increases cell-surface levels of PD-L1 in human cancer cells that express moderate to high levels of PD-L1 (as described above in Example 2), in vitro studies will be performed to investigate the effect of Coenzyme Q10 on PD-L1 expression in murine cancer cell lines as well. These PD-L1 expression studies will include the mouse colon cancer cell line MC38, which has high levels of PD-L1 on the cell surface. Additionally, IFN-β stimulation will be applied to further increase the level of PD-L1 on MC38 cells and the other murine cancer cell lines shown in Table 3 below.

In vitro studies will be performed on the murine cancer cell lines shown in Table 3 to determine the EC₅₀ of Coenzyme Q10, Oxygen Consumption Rate (OCR), Extracellular Acidification Rate (ECAR) and PD-L1 expression. Cancer cell lines from different tissues of origin are chosen based on response to immunotherapeutics, response to the modulators of tumor metabolism, and tissue distribution of Coenzyme Q10. Although Coenzyme Q10 is found in all human cells, the highest concentrations are found in the heart, liver, kidneys and pancreas, organs which have the most metabolically active cells. It is also found in large amounts in the cells of the immune system. See Naini A. et al., 2003, Biofactors 18(1-4):145-52; and canceractive.com/cancer-active-page-link.aspx?n=532. The following cancer cell lines will be evaluated in the in vitro studies.

TABLE 3
Murine cancer cell lines for in vitro evaluation.

| Name | Tissue | Cell type | Disease |
|---|---|---|---|
| EMT 6 | Breast | epithelial | mammary carcinoma |
| B16 F10 | Skin | | melanoma |
| CT26 | Colon | Fibroblast | carcinoma |
| Pan02 | Pancreas | epithelial | adenocarcinoma |
| LL/2 | Lung | | Lewis Lung Carcinoma |
| Renca | Kidney | epithelial | renal adenocarcinoma |
| HEPA 1-6 | Liver | epithelial | hepatoma |
| GL261 | Brain | | Glioma |
| MC38 | Colon | | |

The responsiveness of these mouse cancer cell lines to the immune checkpoint inhibitors anti-PD1 antibody, anti-PD-L1 antibody, and anti-CTLA-4 antibody is shown in Table 4 below. Syngeneic mouse models were inoculated with these mouse cancer cell lines and were treated with antibodies for immune-checkpoint inhibition and tumor volumes were measured. The percentage of tumor growth inhibition (TGI) was calculated to determine the sensitivity of the cancer cell line to the immune checkpoint inhibitor.

TABLE 4
Responsiveness of cancer cell lines to anti-PD1, anti-PD-L1, and anti-
CTLA-4 antibodies as determined by contract research organizations.

| Cell Line | CrownBio | Charles River | Agilux Laboratory |
|---|---|---|---|
| EMT 6 | sensitive to all three immune | moderately | Sensitive to anti-PD1 |
| | checkpoint inhibitors | responsive | and anti-CTLA-4 |
| B16 F10 | moderately sensitive to all immune | Refractory | moderately responsive |
| | checkpoint inhibitors | | to anti-PD1 and anti- |
| | | | PD-L1 |
| CT26 | Moderately responsive to anti-CTLA-4 | Responsive | moderately responsive |
| | and anti-PD but resistance to anti-PD- | | to anti-PD1 and anti- |
| | L1(does not have much PD-L on its | | PD-L1 |
| | surface) | | |
| Pan02 | sensitive to all three immune | No data | No data |
| | checkpoint inhibitors | | |
| LL/2 | Moderately responsive to anti-PD1, | Refractory | No data |
| | resistant to anti-PD-L1, the response to | | |
| | anti-CTLA-4 is unknown | | |
| Renca | Moderately responsive to anti-PD-L1, | Moderately | Responsive only to |
| | resistant to anti-PD1 and anti-CTLA-4 | responsive | Anti-CTLA-4 |
| HEPA 1-6 | No data | No data | No data |
| GL261 | No data | No data | No data |

The responsiveness of these mouse cancer cell lines to modulators of tumor metabolism is shown below.

TABLE 5

| Cell Line | Response to the Modulator of Tumor Metabolism |
|---|---|
| EMT 6 | — |
| B16 F10 | B16F10 demonstrated tumor regression |
| | in response to a combination of |
| | drugs interfering with tumor metabolism |
| | (Schwartz et al., 2013, Invest |
| | New Drugs 31(2): 256-64) |
| CT26 | In CT26 cells, short term starvation |
| | (STS) down-regulated aerobic |
| | glycolysis, and glutaminolysis, while |
| | increasing oxidative phosphorylation. |
| | The STS-dependent increase in both |
| | Complex I and Complex II-dependent |
| | O₂ consumption was associated with |
| | increased oxidative stress and |
| | reduced ATP synthesis. STS potentiated |
| | the effects of Oxaliplatin on the |
| | suppression of colon carcinoma |
| | growth and glucose uptake in both |
| | in vitro and in vivo models (Biachi et al., |
| | 2015, Oncotarget 6(14): 11806-19.) |
| Pan02 | — |
| LL/2 | — |
| Renca | In vivo growth of renal tumors (RENCA) |
| | expressing lower Plasminogen |
| | activator inhibitor-1 levels was inhibited |
| | by stable urokinase. (Jing et al., |
| | 2012, Mol Cancer Res. 10(10): 1271-1281) |
| HEPA 1-6 | Up-regulation of the ATPase Inhibitory |
| | Factor 1 Mediates the Metabolic |
| | Shift of Cancer Cells to a Warburg |
| | Phenotype (Sanchez-Cenizo, et al., |
| | 2010, J Biol Chem. 285(33): 25308-13) |
| GL261 | High fat Ketanic diet has been implicated |
| | for the treatment of glioma. This |
| | therapeutic strategy targets the aerobic |
| | fermentation of glucose (Warburg |
| | effect). (Medenbauer et al., 2015, Nutr |
| | Metab 12: 12.) |

### Example 4—In Vivo Studies to Compare Anti-Tumor Activity of Coenzyme Q10 in Patient-Drived Xenograft Models

Patient derived xenografts (PDX) are created when cancerous tissue from a patient's primary tumor is implanted directly into an immunodeficient mouse. This xenograft model allows the study of different investigative drugs on patient tumors with different profiling such as different mutations or different prior treatment. These xenograft models will be used to test and compare the effects of Coenzyme Q10 alone and/or immune checkpoint modulators alone with a combination of the two on patient derived xenografts, using the methods described herein.

### Example 5—In Vivo Studies to Compare Anti-Tumor Activity of Coenzyme Q10 in Immune-Competent and Immune-Deficient Mice

Previous studies in animal models to determine the anti-tumor efficacy of Coenzyme Q10 were conducted in immune-compromised nude mice in which an immune system was not present. If Coenzyme Q10 is able to increases the anti-tumor activity of the host immune system, an increase in the efficacy of Coenzyme Q10 in immune-competent mice (in which a full immune system is present) would be expected relative to immune-compromised mice. Three different approaches will be used to compare the anti-tumor activity of Coenzyme Q10 in immune-competent and immune-deficient mice.

### 1. Syngeneic Mouse Cancer Cell Lines

The anti-tumor activity (e.g. percentage of tumor growth inhibition) of Coenzyme Q10 will be compared in a syngeneic mouse cancer cell line in immune-compromised mice and immune-competent mice. Three different types of immune-compromised mice will be used for this study, depending on the need for the immune system components: nude mice (athymic mice lacking only T cells), SCID mice (lacking T cells and B cells) or NOD scid gamma (NSG) mice. NSG mice are Il2rg deficient mice lacking several components of the immune system including mature T cells, B cells, and natural killer (NK) cells. NSG mice are also deficient in multiple cytokine signaling pathways, and they have many defects in innate immunity. They are among the most immunodeficient mice that have been developed. See Shultz et al., 2007, Nat. Rev. Immunol. 7 (2): 118-130.

In these studies, the strains of mice that are used for immune-competent and immune-compromised hosts will be different. The different mouse strains may exhibit different tumor growth rates. Accordingly, the percent inhibition of tumor growth by Coenzyme Q10 will be determined in each mouse strain by comparing tumor size in the treatment group to tumor size in the control group in each strain. The percentage of tumor growth inhibition will be used to compare the efficacy of Coenzyme Q10 in inhibiting tumor growth among different mouse strains.

### 2. Rag-1 Deficient Mice

RAG-1 is a V(D)J recombination activation gene that is thought to activate or catalyze the V(D)J recombination reaction of immunoglobulin and T cell receptor genes. RAG-1-deficient mice have small lymphoid organs that do not contain mature B and T lymphocytes. The immune system of the RAG-1 mutant mice can be described as that of nonleaky SCID mice. See Mombaerts et al., 1992, Cell 68(5):869-77, which is incorporated by reference herein in its entirety. Rag-1 deficient mice were developed in the C57 mouse genetic background. Therefore, Rag-1 deficient mice and C57 mice can be used side-by-side for this comparison. The B16 F10 cell line described is a mouse melanoma cell line generated from C57 mice, and thus is syngeneic to C57. Accordingly, B16 F10 cells will be used for this study.

### 3. Irradiated Mice

Immune cells and cytokines respond differently to low and high doses of irradiation. Although irradiation causes apoptosis in immune cells, the levels of different immune cells will vary after irradiation. Therefore, irradiation provides a method for developing mice with varied levels of particular types of immune cells, such as natural killer (NK) cells and dendritic cells (DCs). See Bogdandi et al., 2010, Radiat Res. 174(4):480-9, which is incorporated by reference herein in its entirety.

Mouse cancer cell lines including those described above in Example 3 (Tables 3-5) will be evaluated in syngeneic mouse models to compare the anti-tumor activity of Coenzyme Q10 in immune-competent and immune-deficient mice. The cancer cell lines will be injected into the mice subcutaneously or orthotopically (i.e. in the anatomical position corresponding to the original tumor). Additional particular examples of syngeneic mouse models for evaluation of Coenzyme Q10 anti-tumor activity are provided in Table 6 below.

4. Humanized Mouse (NSG from Jackson Laboratory)

NSG™ humanized mice are extremely immunodeficient. The mice carry two mutations on the NOD/ShiLtJ genetic background; severe combined immune deficiency (scid) and a complete null allele of the IL2 receptor common gamma chain (IL2rg^{null}). The scid mutation is in the DNA repair complex protein Prkdc and renders the mice B and T cell deficient. The IL2rg^{null} mutation prevents cytokine signaling through multiple receptors, leading to a deficiency in functional NK cells. The severe immunodeficiency allows the mice to be humanized by engraftment of human CD34+ hematopoietic stem cells (HSC), peripheral blood mononuclear cells (PBMC), patient derived xenografts (PDX), or adult stem cells and tissues. The immunodeficient NSG™ mice enable research in human immune function, infectious disease, diabetes, oncology, and stem cell biology” (jax.org/strain/005557). These animals can be engrafted with human peripheral blood mononuclear cell (PBMC) or human CD34+ cells in order to generate a human immune system in the mouse body.

TABLE 6
Exemplary syngeneic mouse models.

| | Subcutaneous | |
|---|---|---|
| | Models | Orthotopic Models |
| Cancer Type | Cell Line | Cell Line |
| Breast | 4T1, EMT-6, JC | 4T1, EMT6 |
| Bladder | MBT-2 | |
| Colon | Colon26, CT26, | |
| | MC38 | |
| Renal | Renca | |
| Liver | H22, Hepa1-6, | |
| | Yoshida | |
| Lung | LL/2, KLN 205, | |
| | KLN 206, Lewis | |
| | Lung, Madison 109 | |
| Pancreatic | Pan02 | |
| Prostate | RM-1 | |
| Melanoma | B16BL6, B16F10, | |
| | Cloudman S91 | |
| Sarcoma | EHS | |
| Glioma | | GL261 |

Bioluminescent syngeneic mouse models, which allow the study of clinically relevant metastatic invasion, metastatic lesions in secondary organs, and the evaluation of agents to target this metastasis, will also be evaluated.

The mice will be administered Coenzyme Q10 by continuous infusion (CI) or intraperitoneal injection (IP) at various dosages. Exemplary treatment groups are provided below in Table 7.

TABLE 7
Exemplary treatment groups for comparison of immune-competent (e.g. Balb/c) and
immune-compromised (e.g. nude) cancer mouse models treated with Coenzyme Q10.

| | Number | Test | | Dose | Total Dose | Route of | |
|---|---|---|---|---|---|---|---|
| Group | of Animals | Article | Dose | Schedule | Per Day | Admin. | Mouse Strain |
| 1 | 10 | vehicle | NA | TBD | | | Balb/c |
| | | (control) | | | | | C57bl/6 |
| | | | | | | | Humanized |
| | | | | | | | mouse |
| 2 | 10 | CoQ10 | 75 mg/kg | 1X/day | 75 mg/kg | CI, PO | Balb/c |
| | | | | | | or IP | C57bl/6 |
| | | | | | | | Humanized |
| | | | | | | | mouse |
| 3 | 10 | CoQ10 | 25 mg/kg | 3X/day | 75 mg/kg | CI, PO | Balb/c |
| | | | | | | or IP | C57bl/6 |
| | | | | | | | Humanized |
| | | | | | | | mouse |
| 4 | 10 | vehicle | NA | TBD | | | nude |
| | | (control) | | | | | |
| 5 | 10 | CoQ10 | 75 mg/kg | 1X/day | 75 mg/kg | CI, PO | nude |
| | | | | | | or IP | |
| 6 | 10 | CoQ10 | 25 mg/kg | 3X/day | 75 mg/kg | CI,PO | nude |
| | | | | | | or IP | |

CI = continuous infusion;
IP = intraperitoneal injection;
PO = oral administration.

A satellite group will be added to Balb/c animals for FACS analysis. The mice will be evaluated by the following parameters shown below in Table 8.

TABLE 8
Study endpoints for comparison of immune-competent
and immune-compromised cancer mouse models.

| | |
|---|---|
| Study Activities/ | Daily for morbidity |
| Endpoints: Clinical | |
| Observations/health | |
| monitoring | |
| Body Weights | Twice weekly |
| Tumor Weights/Measurement | Twice weekly |
| Flow Cytometry | All animals: Flow Cytometry will be |
| | performed on blood, spleen, lymph |
| | nodes and tumor to assess both |
| | TILs and TAMs |
| Blood collection | All animals: Terminal cardiac stick |
| | Whole blood to be processed for flow |
| | cytometry and toxicity |
| Necropsy | All animals: Collect tumor, lymph |
| | nodes, spleen, liver, lung |
| | Tissues going to flow cytometry will be |
| | split in half with one half processed for |
| | flow cytometry and one half placed part |
| | into 10% NBF and another part snap |
| | frozen in OCT. |
| | All other tissues will be placed half into |
| | 10% NBF and half snap frozen in OCT |
| Histology | Tissue will be processed for paraffin |
| | block and frozen for future IHC |

### FACS and Biomarker Analysis

In addition to the study endpoints described above in Table 8, the murine tumor immune environment will be evaluated using FACS analysis of the following immune cells and markers.

TABLE 9
Immune cells and markers for FACS analysis.

| Immune Cells | | Marker (Mouse) |
|---|---|---|
| B cells | | CD45R/B220 |
| T cells | Total T Cells | CD3 |
| | Helper T Cells | CD4 |
| | Cytotoxic T Cells | CD8 |
| | Regulatory T Cells | CD25, FOXP3 |

| | |
|---|---|
| Dendritic Cell | CD11c, CD123 |
| NK Cells | CD335 |
| Macrophage | CD11b, F4/80, Iba1 |
| Monocytes | CD11b |
| Neutrophil | Ly-6G/C |
| MDSC | CD11b, Ly-6G, Gr-1 |
| Check-point | PD-1, PD-L1, GAta3, CTLA-4 |
| Additional Biomarkers | CD8B, CD247, CFL1, S100A8, |
| | ADD1, NAE1, GOLPH3, BAG6, |
| | LSP1, APBB1IP, MIEN1, DCTN1, |
| | CDC42, PPP5C, SPTBN1, |
| | ARHGEF2, ELMO1, CSK, VAMP8, |
| | PRKAR1A, MAP4K2, CTSD, LYN, |
| | ZAP70, FAM21C, AP1S1, RRAS, |
| | SEMA4D, SNAP23, HLA.E, FCER1G, |
| | RAB3D, CD14, TREML1, EHD4, |
| | ERAP1, NCKAP1L, TAPBP, C8A, |
| | HLA.DRB5, ITGA6, SNTB1, CD5L, |
| | HV101, IGHM |

Frozen tumor tissue from the mice will also be further analyzed for the following tumor microenvironment markers: IL-6, IFN-γ, IL-17, TNF-α, TGF-β and IL-10.

Innovative immunotherapeutics have entered the clinic largely based on the recognition that immune cells and their mediators may both hinder and foster tumor development. It is of great interest to investigate what influence cancer cell killing targets have on the immune cells in the tumor microenvironment. Therefore, the following immune cell biomarkers will also be analyzed: CD8B, CD247, CFL1, and S100A8.

The results of the studies are expected to demonstrate that CoQ10 shows greater efficacy in immune-competent mice as compared to immune-deficient mice.

### Example 6—In Vivo Studies to Evaluate Co-Administration of Coenzyme Q10 and Immune Checkpoint Inhibitors

Three well-characterized immune checkpoint inhibitors will be evaluated in combination therapy with Coenzyme Q10 in mouse cancer models: anti-PD-L1, anti-PD1, and anti-CTLA-4.

Anti-PD-L1: Atezolizumab (Tecentriq™) is an anti-PD-L1 monoclonal antibody that was approved by the FDA in May 2016 for the treatment of bladder cancer. PD-L1 is produced by both immune cells and tumor cells. Many tumor cells have developed the ability to express high levels of PD-L1 to suppress immune response against themselves. For evaluation of anti-PD-L1 antibody, it is important to identify the level of PD-L1 on the surface of the mouse cancer cell lines that are chosen for the in vivo studies. Data from human cancer cell lines indicate that Coenzyme Q10 treatment increased cell surface levels of PD-L1 in cells that express moderate to high levels of PD-L1 before treatment, but did not induce cell surface expression of PD-L1 in cells that do not have PD-L1 on their surface before treatment. Accordingly, additional experiments confirming the regulation of surface expression of PD-L1 in response to Coenzyme Q10 in mouse cancer cell lines will be conducted as described above in Examples 2 and 3.

Anti-PD1: Pembrolizumab (Keytruda™) is an anti-PD1 monoclonal antibody that has been approved by the FDA for treatment of metastatic melanoma. Most mouse syngeneic models respond to anti-PD1 antibody, but the level of response varies. Nivolumab (Opdivo™) is a humanized IgG4 anti-PD1 monoclonal antibody that has been FDA approved for patients with metastatic melanoma and previously treated advanced or metastatic non-small-cell lung cancer. See Sharma et al., 2015, Cell 161: 205-214, which in incorporated by reference herein in its entirety.

Anti-CTLA-4: Ipilimumab (Yervoy™) is an anti-CTLA-4 monoclonal antibody that also has been approved by the FDA for the treatment of metastatic melanoma. It is actively being investigated for treatment of other cancers such as non-small-cell lung cancer (NSCLC).

Each of these antibodies will be tested in combination with Coenzyme Q10 as described below in Table 10.

TABLE 10
Treatment groups for evaluation of co-administration of Coenzyme Q10 and
immune checkpoint inhibitors (e.g anti-PD1, anti-PD-L1 and anti-CTLA-4).

| | Number | Test | | Dose | Total Dose | Route of | |
|---|---|---|---|---|---|---|---|
| Group | of Animals | Article | Dose | Schedule | Per Day | Admin. | Mouse Strain |
| 1 | 10 | vehicle | NA | TBD | | | Balb/c, |
| | | (control) | | | | | C57bl/6 |
| | | | | | | | Humanized |
| | | | | | | | mouse |
| 2 | 10 | CoQ10 | 25 mg/kg | 2X/day | 50 mg/kg/day | IP or PO | Balb/c, |
| | | | | | | | C57bl/6 |
| | | | | | | | Humanized |
| | | | | | | | mouse |
| 3 | 10 | CoQ10 | 100 mg/kg | 2X/day | 200 mg/kg/day | IP or PO | Balb/c, |
| | | | | | | | C57bl/6 |
| | | | | | | | Humanized |
| | | | | | | | mouse |
| 3 | 10 | Anti-PD1 | 10 mg/kg | every | 10 mg/kg | IV or ip | Balb/c, |
| | | | | three | | | C57bl/6 |
| | | | | days | | | Humanized |
| | | | | | | | mouse |
| 4 | 10 | CoQ10 | 25 mg/kg | 2X/day | 50 mg/kg and | ip or po | Balb/c, |
| | | and | and | and | 10 mg/kg | and | C57bl/6 |
| | | anti-PD1 | 10 mg/kg | every | | iv or ip | Humanized |
| | | | | three | | | mouse |
| | | | | days | | | |
| 5 | 10 | CoQ10 | 100 mg/kg | 2X/day | 200 mg/kg and | ip or po | Balb/c, |
| | | and | and | and | 10mg/kg | and | C57bl/6 |
| | | anti-PD1 | 10 mg/kg | every | | iv or ip | Humanized |
| | | | | three | | | mouse |
| | | | | days | | | |

A satellite group will be added to Balb/c animals for FACS analysis. The mice will be evaluated according to the study endpoints described above in Table 8 and the FACS and biomarker analysis described above in Example 5.

### Example 7—Effects of Coenzyme Q10 on Frequency, Viability, Cytokine Production, and Immune Checkpoint Protein Expression of PHA-Stimulated and Non-Stimulated Human Immune Cells from Healthy Donors

Coenzyme Q10 has a unique mechanism of action that effectuates an anti-Warburg switch in cancer cell metabolism and activation of apoptosis. Given the observed central role of Coenzyme Q10 in regulating mitochondrial function in cancer cells, the ability of Coenzyme Q10 to modulate immune cells and their functionality was determined. Specifically, the effects of Coenzyme Q10 on the frequency and viability of human peripheral blood mononuclear cells (PBMC) were investigated to elucidate the immuno-metabolic mechanism of Coenzyme Q10. In addition, the effect of Coenzyme Q10 on immune cell function was evaluated by measuring T cell proliferation and a panel of cytokines released by the cells.

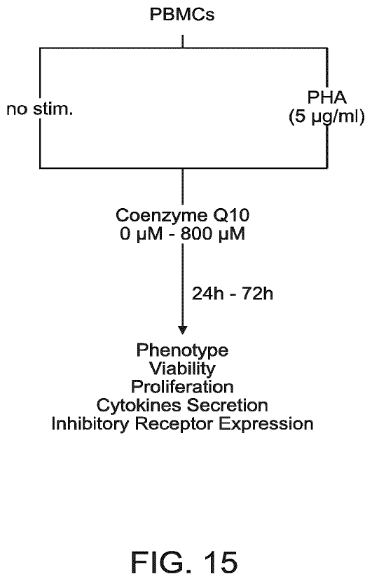

FIG. 15 shows a schematic representation of an ex vivo peripheral blood mononuclear cell (PBMC) model used to investigate the effect of Coenzyme Q10 on human immune cells. PBMCs isolated from healthy human donor leukopaks were isolated and cryopreserved. To study the effect of Coenzyme Q10, cells were thawed, rested overnight and treated with or without phytohemagglutinin (PHA). PHA is a lectin protein found in plants which activates T cells by inducing mitosis. Various concentrations of Coenzyme Q10 (0, 12.5, 50, 200, 400 or 800 μM) were added to the cells at the same time. 24 hours to 72 hours post-treatment, frequency and viability of immune cell subpopulations was evaluated, as well as proliferative potential, cytokine secretion, and inhibitory receptor surface expression.

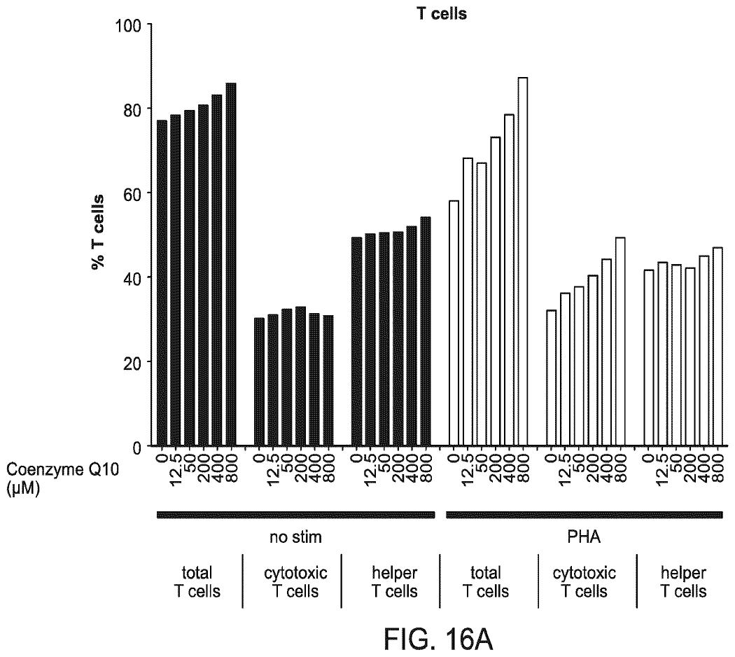

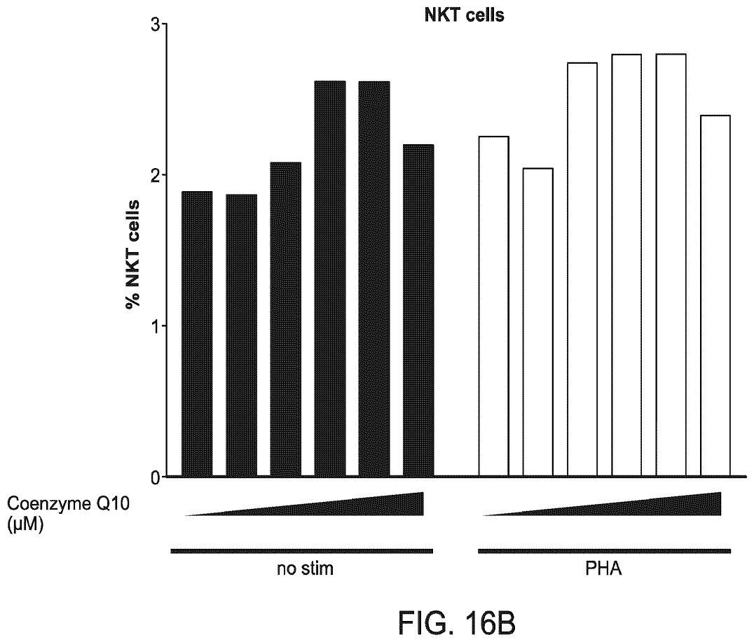

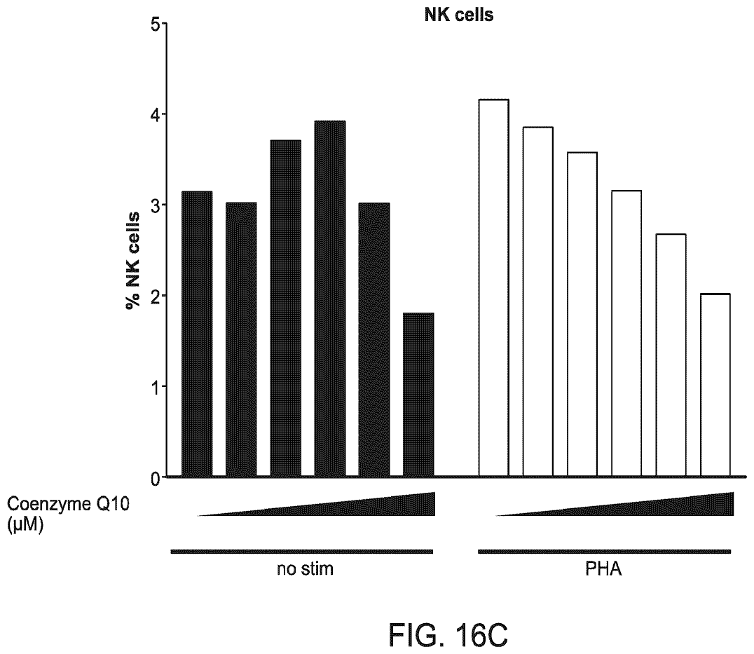

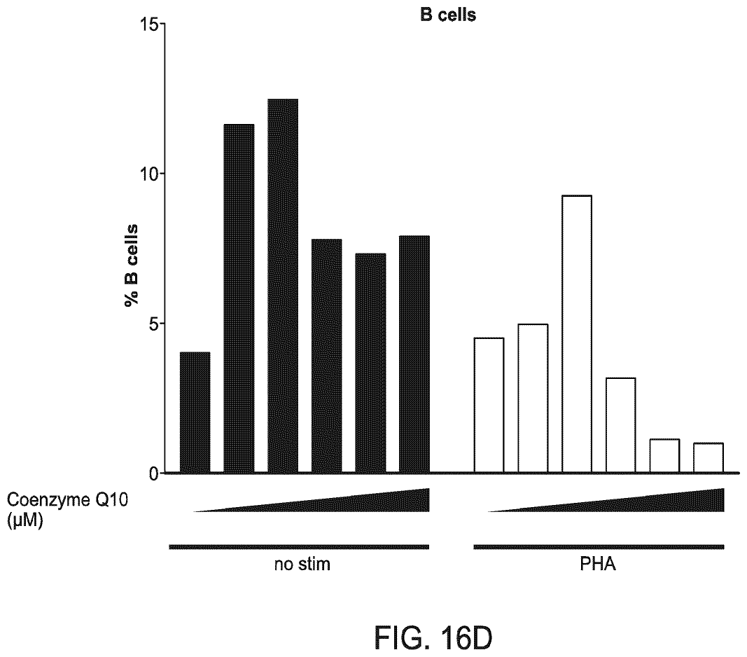

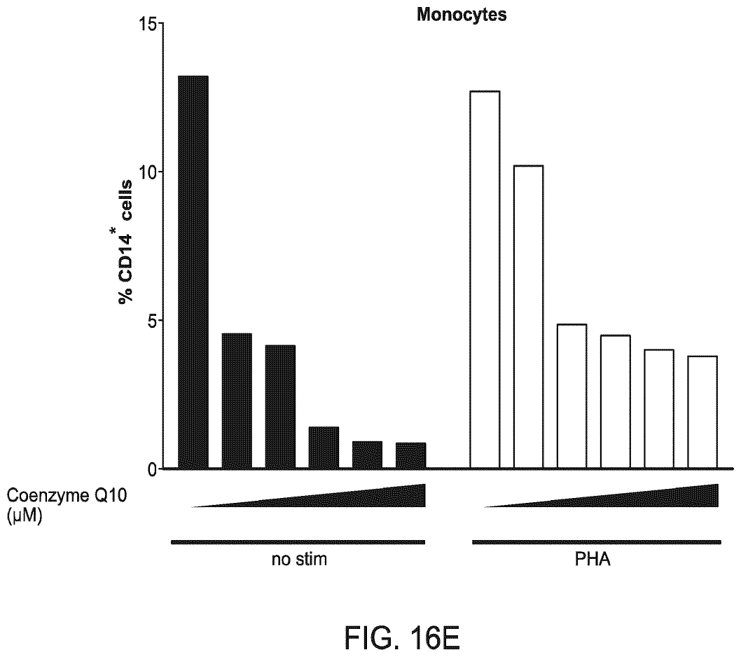

The frequency of different immune cell populations within PHA stimulated or unstimulated human PBMCs concurrently treated with Coenzyme Q10 (0, 12.5, 50, 200, 400 or 800 μM) was evaluated by flow cytometry. 24 hours post treatment, PBMCs were analyzed for the surface markers CD3/CD8, CD3/CD4, CD3/CD56, or CD19/CD14. Frequency of immune cell subtypes was quantified by percentage of cells gated for T cells (CD3/CD8/CD4), natural killer T cells (NKT), natural killer (NK) cells, B cells, and monocytes. Cells from 5 healthy donors were tested. As shown in FIG. 16A, Coenzyme Q10 increased the frequency of total T cells, cytotoxic T cells, and helper T cells in a dose dependent manner, with a greater effect observed for PHA-stimulated cells relative to unstimulated cells. The effects of Coenzyme Q10 on the frequency of NKT cells, NK cells, B cells monocytes are shown in FIGS. 16B-16E, respectively.

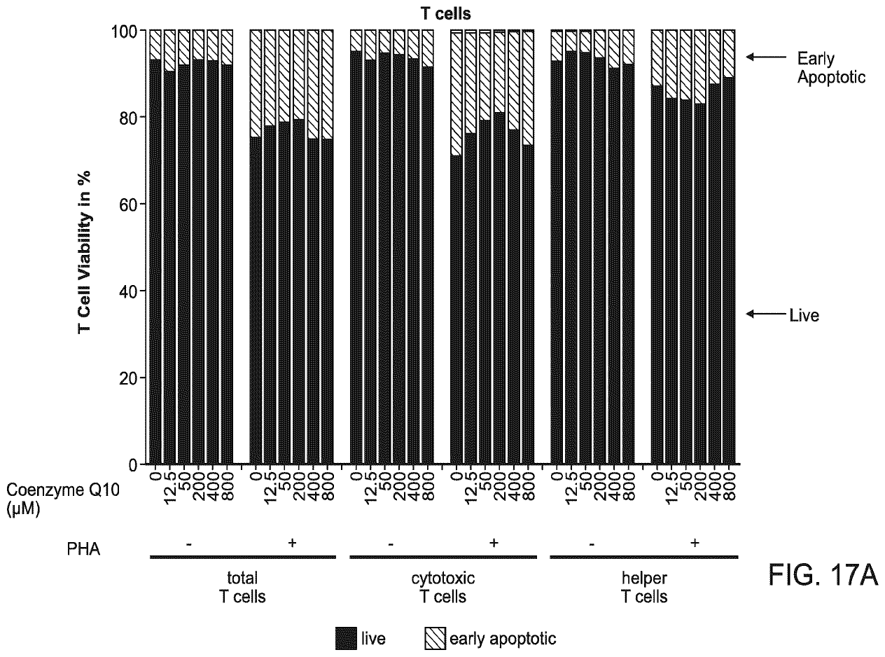

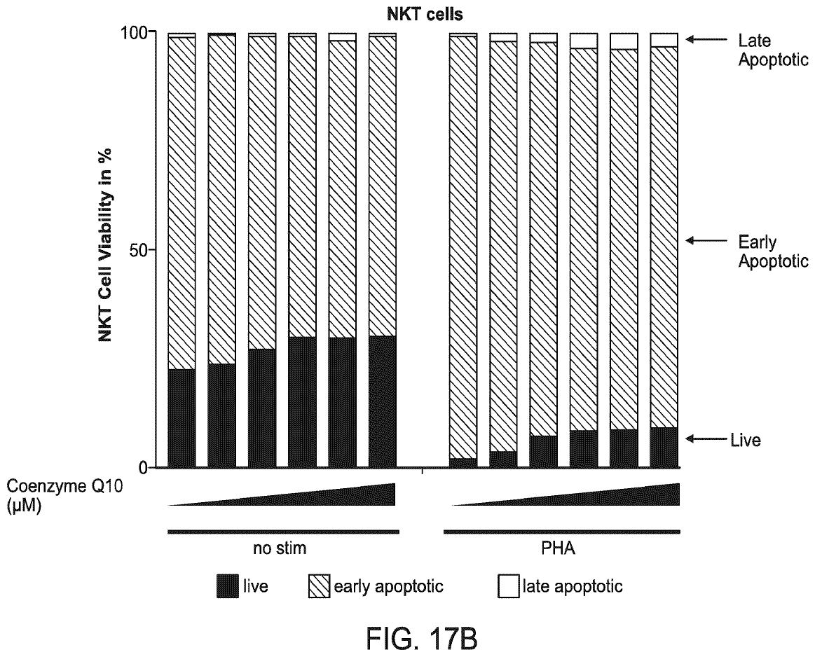

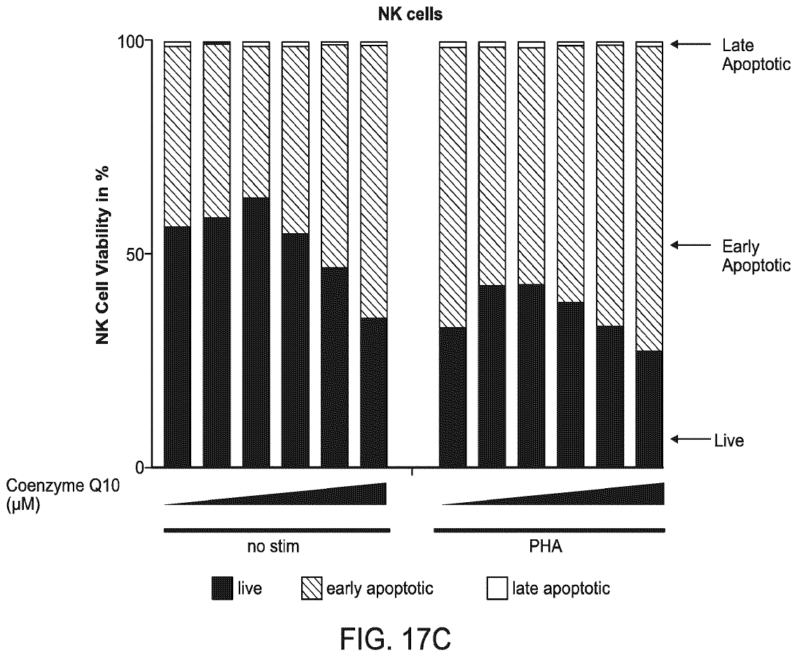

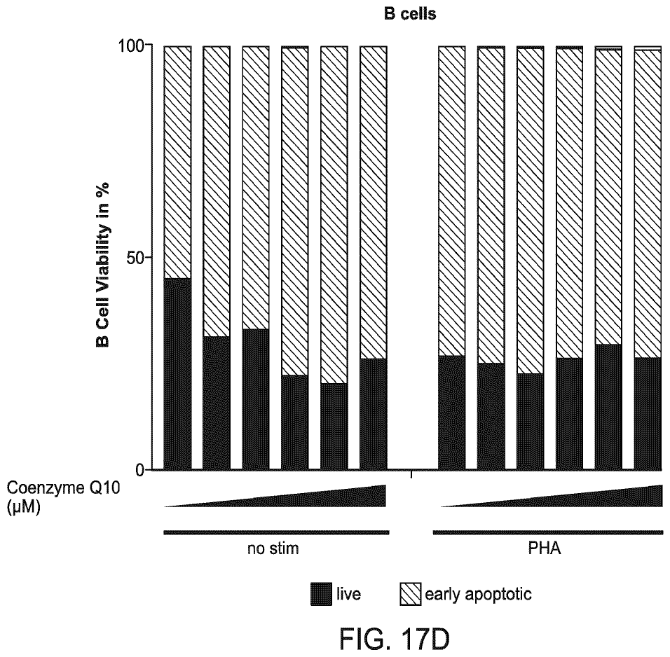

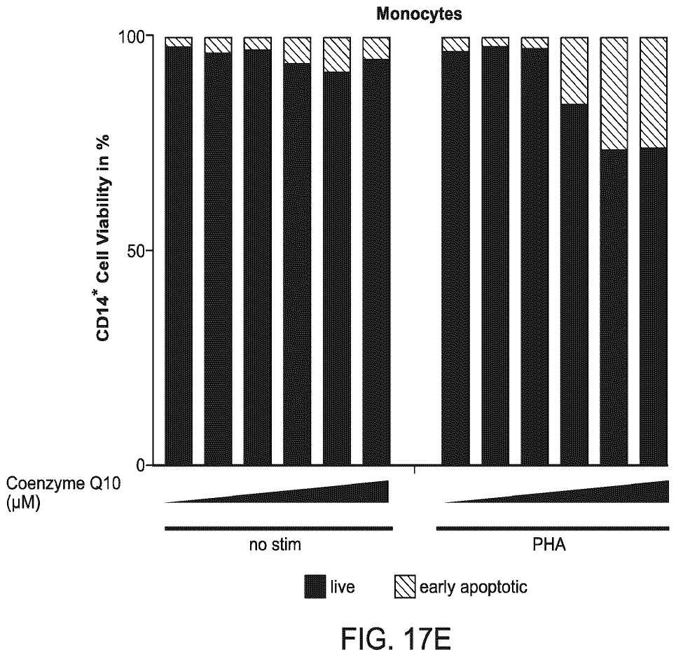

The viability of human immune cell subpopulations within PHA-stimulated or unstimulated PBMCs concurrently treated with Coenzyme Q10 was also determined. Cells were treated with 0, 12.5, 50, 200, 400 or 800 μM Coenzyme Q10 for 24 hours. Cell populations and viability was determined by flow cytometry using combinational staining of surface markers using Annexin V/7 AAD stains. Cells from 5 healthy donors were evaluated. Total, cytotoxic and helper T cell viability after treatment with increasing Coenzyme Q10 concentrations show T cell viability increased in response to Coenzyme Q10. FIG. 17A. Viability of CD3+/CD8+ cells (cytotoxic T cells), CD3+/CD4+ cells (helper T cells), CD3−/CD56+ cells (NK cells), CD3+/CD56+(NKT), CD19+(B cells), and CD14+ cells (monocytes) is shown in FIGS. 17B-17E, respectively.

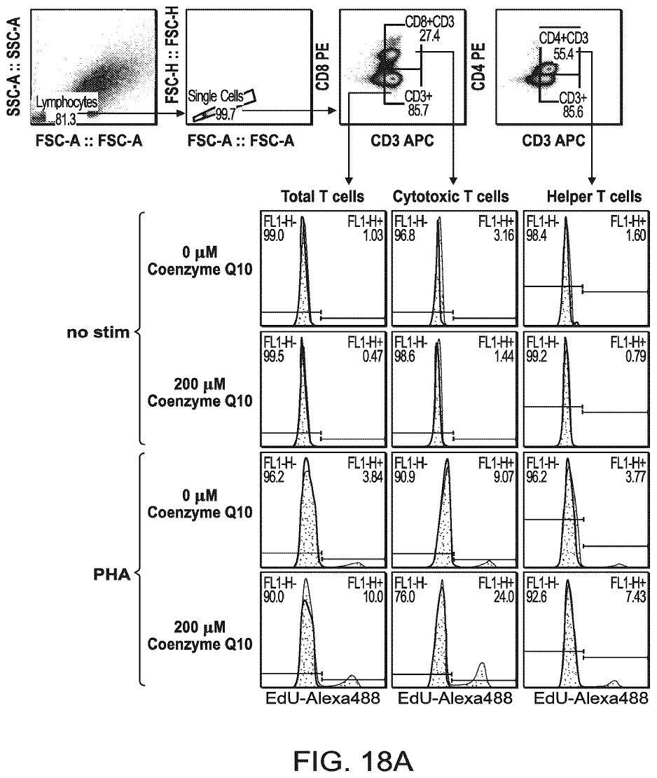

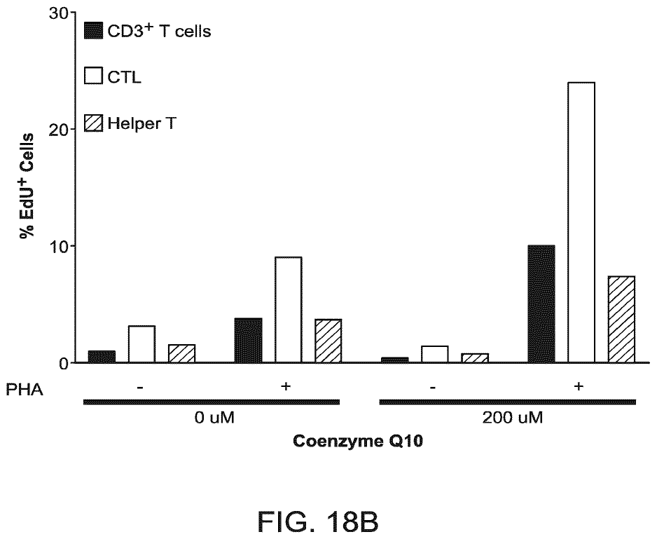

Proliferation of T cells was assessed by flow cytometry using Click-iT® EdU technology (Thermo Fisher Scientific, Waltham, MA), a proliferation assay that is optimized for fluorescence microscopy applications. In this assay, the modified thymidine analogue EdU is incorporated into newly synthesized DNA and fluorescently labeled with a bright, photostable Alexa Fluor® dye. PBMCs were obtained from three different human donors. PBMCs were incubated with or without PHA for 72 hours while concurrently treated with Coenzyme Q10 (200 μM). 10 μM of EdU was added for the final 18 hours and stained with Invitrogen Alexa Fluor 488 piclyl azide according to the manufacturer's protocol. Cells were then stained with surface marker antibodies for CD3/CD8, or CD3/CD4 to identify cytotoxic T cells or helper T cells, respectively. Cells were then analyzyed by flow cytometry applying gating strategy. FIG. 18A shows histogram plots demonstrating clear separation of cells in S phase (DNA synthesis, including EdU incorporation) and cells in either G2/M or G0/G1. FIG. 18B shows a graphic display of T cell proliferation values acquired from the histogram plots.

In summary, the data in FIGS. 16-18 show that Coenzyme Q10 dose-dependently increased the frequency and viability of human CD3⁺ T cells, and increased proliferation of PHA-activated cytotoxic T cells.

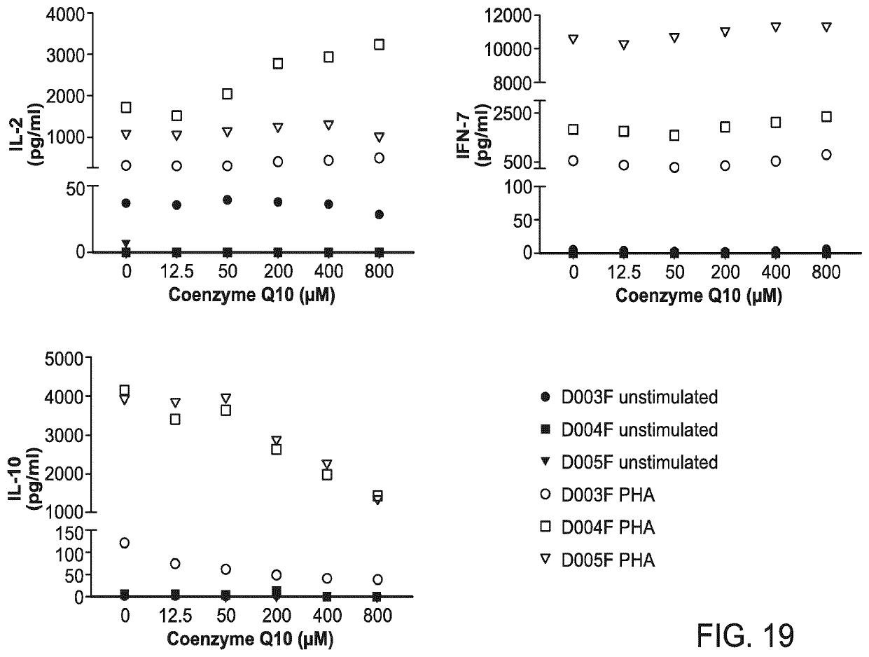

Cytokines were measured in the PBMCs according to the manufacturer's protocol for R&D Quantikine ELISA kits (R&D Systems, Inc., Minneapolis, MN) specific to each cytokine. PBMCs were collected from 3 donors (D003F, D004F and D005F). FIG. 19 shows the levels of the cytokines IL-2, interferon-7 (IFN-7) and IL-10 in supernatants of PHA-stimulated and rested PBMCs concurrently treated with various concentrations of Coenzyme Q10. These data show that Coenzyme Q10 altered the cytokine milieu during PHA activation, i.e. levels of the effector cytokines (IL-2, IFN-7) were increased, while the suppressor cytokine IL-10 was dose-dependently decreased.

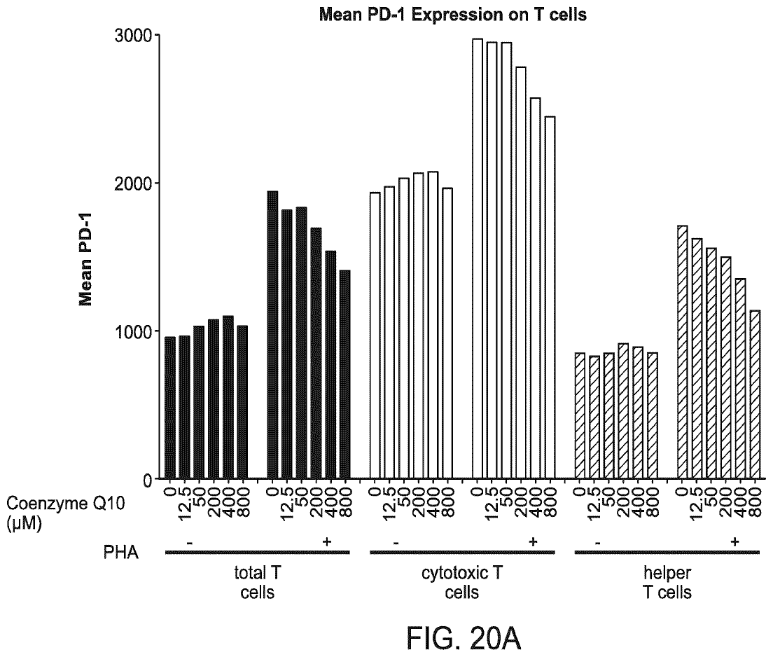

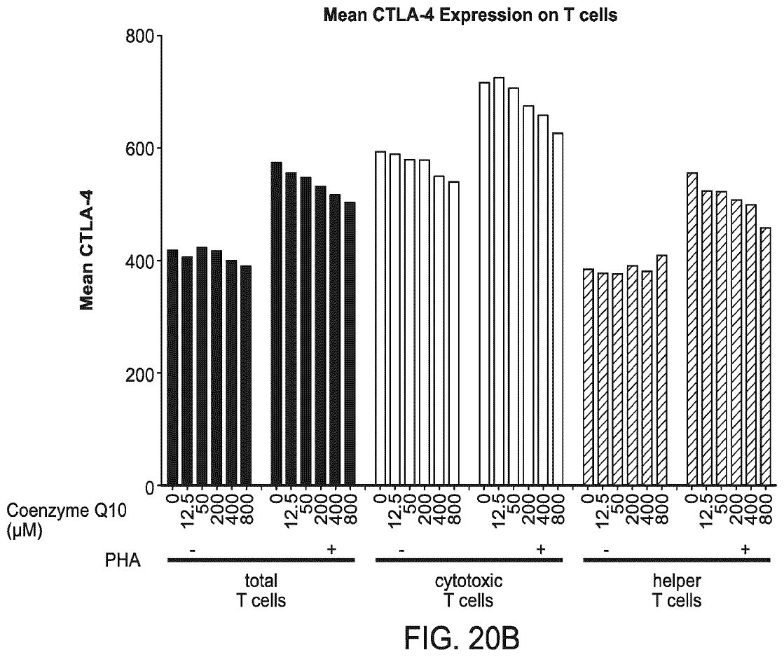

Expression of the inhibitory receptor proteins PD-1 and CTLA-4 on the surface of T cells within PBMCs was also determined. The cells were treated with Coenzyme Q10 for 24 hours. Expression of immune checkpoint receptors was measured by staining cells with phenotypic markers for CD3/CD8, or CD3/CD4 in combination with antibodies against PD-1 or CTLA-4. Live cells were identified as 7 AAD negative lymphocytes followed by T cell phenotype characterization of total CD3+ T cells, cytotoxic T cells, or helper T cells. PD-1 or CTLA-4 cell surface expression was measured as mean fluorescence intensity on live T cells. Cells from three donors were tested. As shown in FIGS. 20A and 20B, Coenzyme Q10 decreased PD-1 and CTLA-4 expression on the surface of PHA-stimulated T cells in a dose dependent manner.

Considered together, these data show that Coenzyme Q10 has a direct effect on immune cells and their functionality. Coenzyme Q10 supports cell proliferation of T cells and effector function of adaptive immune cells indicating that the efficacy of Coenzyme Q10 in cancer treatment may result from both direct effects on tumors and its immuno-regulatory function.

### Example 8—Effects of Coenzyme Q10 on Viability and Immune Checkpoint Protein Expression of PHA-stimulated and Non-stimulated Murine Immune Cells

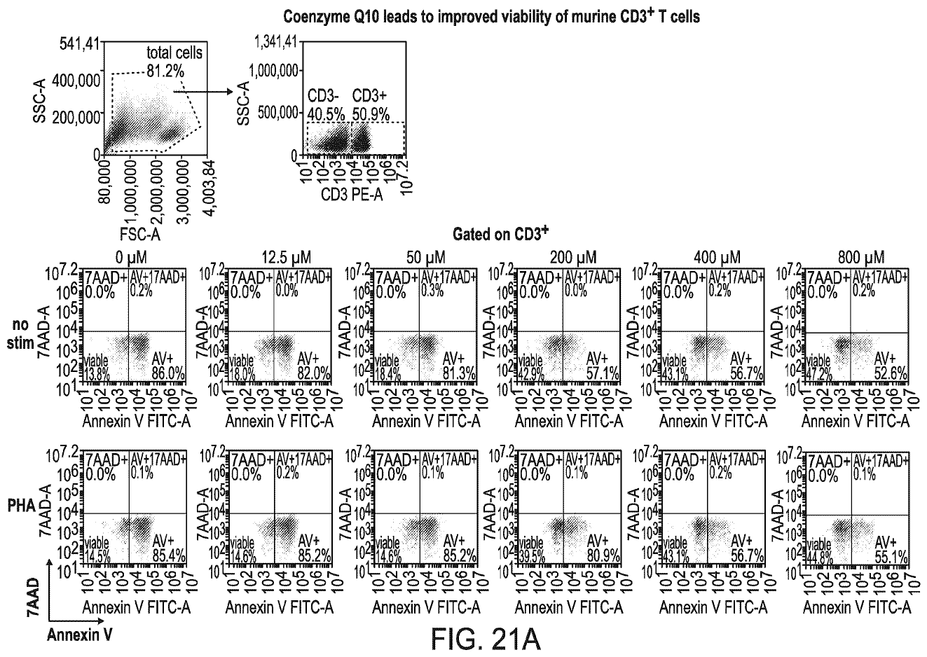

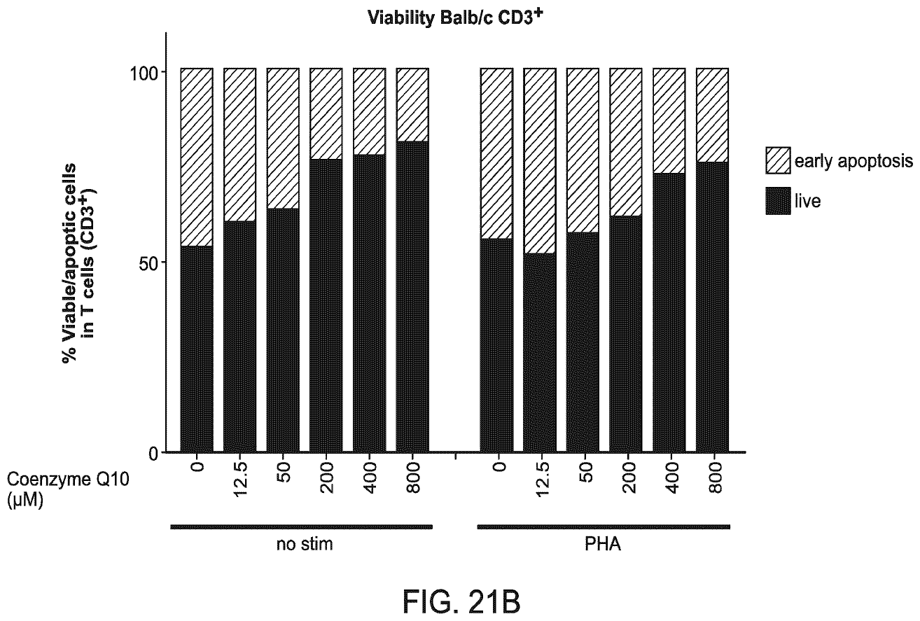

The effects of Coenzyme Q10 on murine immune cells were also evaluated to determine whether Coenzyme Q10 has similar effects as those described above for human immune cells. For example, the viability of murine CD3 positive T cells within PHA-stimulated or unstimulated Balb/c PBMCs was determined. Cells were concurrently treated with Coenzyme Q10 (0, 12.5, 50, 200, 400 or 800 μM) for 24 hours and analyzed by flow cytometry using surface marker antibody for αCD3 and viability stains Annexin V/7AAD. CD3 positive and CD3 negative cell populations were identified within total cell population excluding debris, and viability was determined by plotting Annexin V-FITC vs. 7AAD. Two experiments using two different pools of Balb/c PBMCs and one experiment using C57Bl/6 PBMCs were conducted. As shown in FIGS. 21A and 21B, Coenzyme Q10 increased the viability of CD3 positive murine T cells in a dose-dependent manner for both non-stimulated and PHA-stimulated cells.

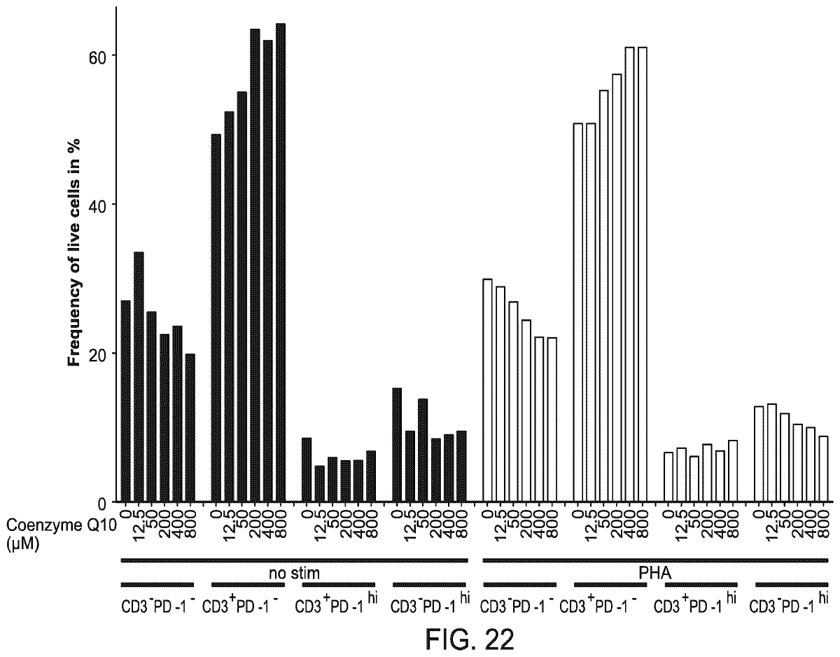

The frequency of PD-1 expressing cells within PHA-stimulated or unstimulated murine Balb/c PBMCs was also evaluated. Cells were concurrently treated with Coenzyme Q10 (0, 12.5, 50, 200, 400 or 800 μM) for 24 hours and evaluated by flow cytometry using surface marker antibody for αCD3 and viability stains Annexin V/7AAD. Viable cells were identified by plotting Annexin V vs. 7 AAD, and gated viable cells were subjected to CD3 vs. PD-1 staining. Two experiments using two different pools of Balb/c PBMCs and one experiment using C57Bl/6 PBMCs were conducted. As shown in FIG. 22, Coenzyme Q10 increased the frequency of PD-1 negative CD3 positive T cells in a dose dependent manner for both unstimulated and PHA-stimulated cells.

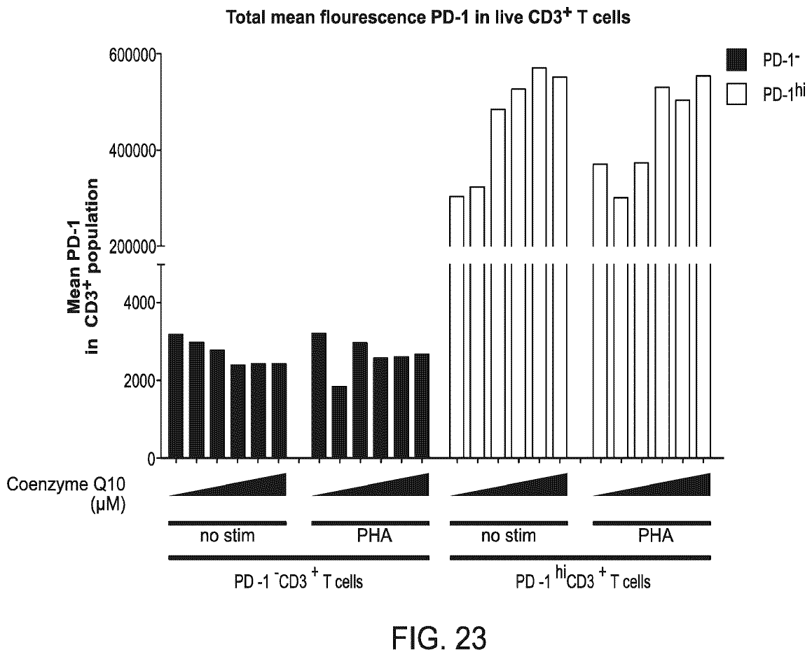

PD-1 surface expression on CD3 positive T cells within PHA stimulated or unstimulated Balb/c PBMCs was also evaluated. Cells were treated with Coenzyme Q10 (0, 12.5, 50, 200, 400 or 800 μM) for 24 hours and PD-1 expression was determined by gating live CD3 positive T cells. Mean fluorescence intensity values were evaluated in histogram plots for PD-1. Two experiments using two different pools of Balb/c PBMCs and one experiment using C57Bl/6 PBMCs were conducted. As shown in FIG. 23, Coenzyme Q10 does not affect PD-1 expression of PD-1 negative gated CD3⁺ T cells, but leads to increased PD-1 levels on PD-1 high expressing gated CD3⁺ T cells. These results indicate that Coenzyme Q10 increases expression of PD-1 in T cells that were already expressing PD-1 at the time of treatment, but does not induce PD-1 expression in cells that are not expressing PD-1.

### Example 9—Effects of Coenzyme Q10 on Viability and PD-L1 Expression of Mouse Syngeneic Tumor Cell Lines In Vitro

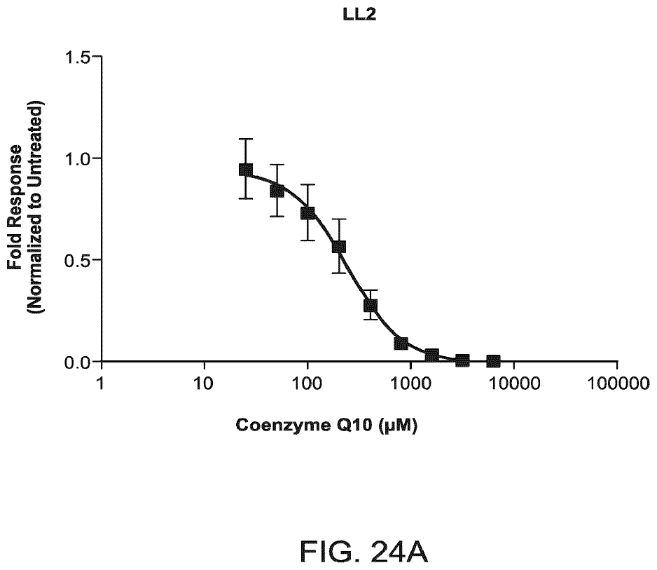

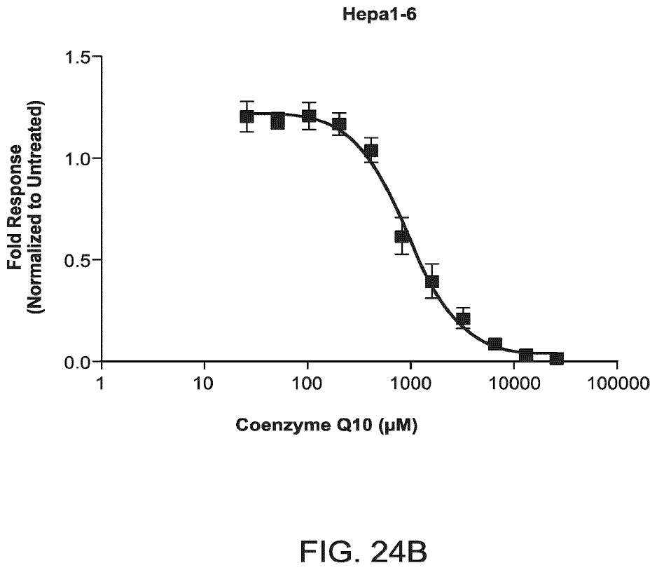

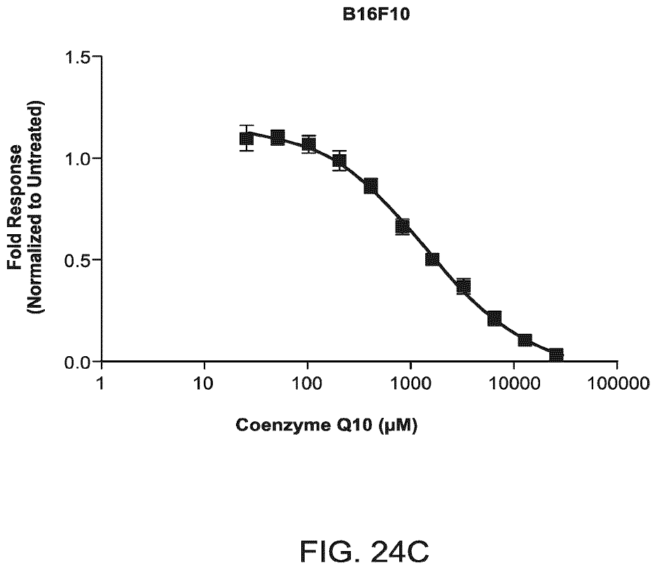

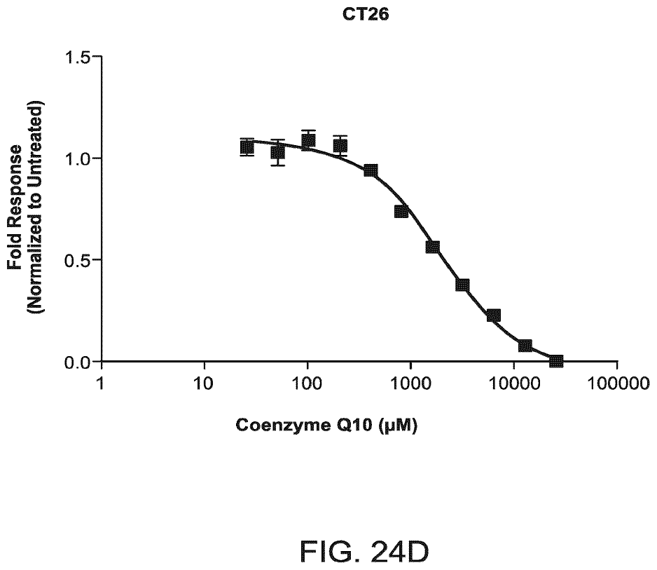

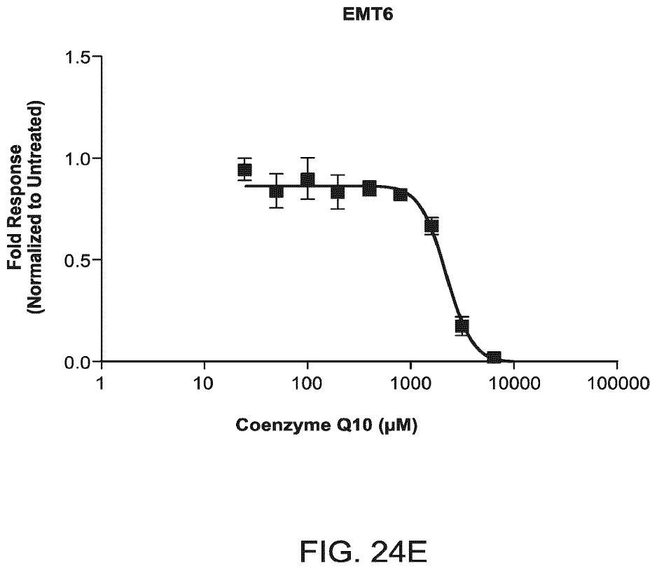

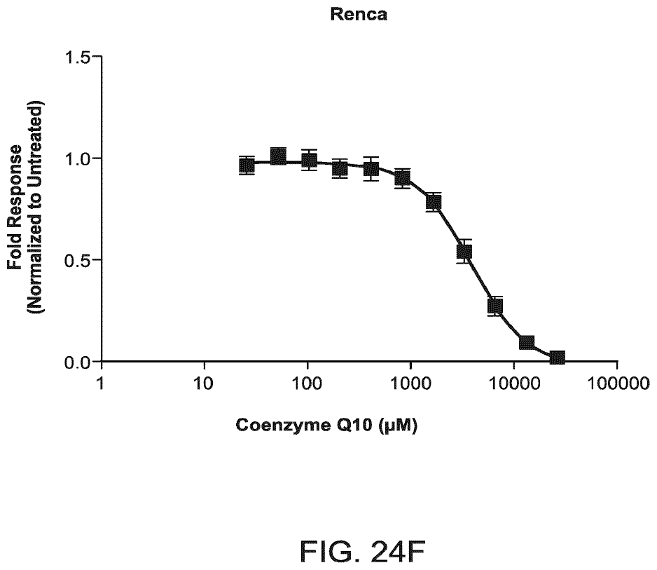

The sensitivity of mouse syngeneic tumor cell lines to Coenzyme Q10 was evaluated in vitro. Six mouse syngeneic tumor cell lines from different tissue types were exposed to increasing concentrations of Coenzyme Q10 (0-25 mM) at 37° C. for 72 hours. Cell viability was measured using CellTiter-Fluor kit (Promega, Madison, WI). Graphs and IC₅₀ values were calculated using GraphPad Prism using data for at least three independent experiments. Mouse syngeneic tumor cell lines evaluated were Lewis lung carcinoma (LL2), hepatoma (Hepa1-6) skin melanoma (B16F10), colon cancer (CT26), mammary gland adenocarcinoma (EMT6/P), and renal adenocarcinoma (Renca). As shown in FIGS. 24A-24F, Coenzyme Q10 reduced viability of all of the tumor cell lines in a dose dependent manner. The IC₅₀ for Coenzyme Q10 for each cell line is shown in Table 11 below.

TABLE 11
IC₅₀ for Coenzyme Q10 in mouse syngeneic tumor cell lines.

| | | IC₅₀ | 95% Confidence |
|---|---|---|---|
| Cell Line | Type of Cancer | (μM) | Interval |
| LL/2 | Lewis Lung Carcinoma | 237.3 | 150.8 to 373.3 |
| Hepa1-6 | Hepatoma | 911.9 | 737.7 to 1127 |
| B16F10 | Skin Melanoma | 1360 | 1007 to 1837 |
| CT26 | Colon Cancer | 1914 | 1502 to 2438 |
| EMT6/P | Mammary Gland | 2202 | 1872 to 2590 |
| | Adenocarcinoma | | |
| Renca | Renal Adenocarcinoma | 3748 | 2919 to 4812 |

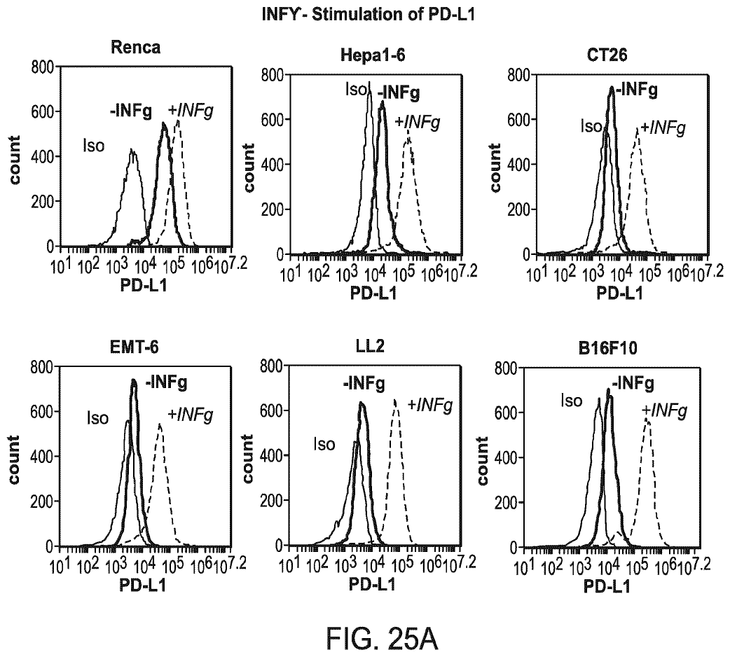

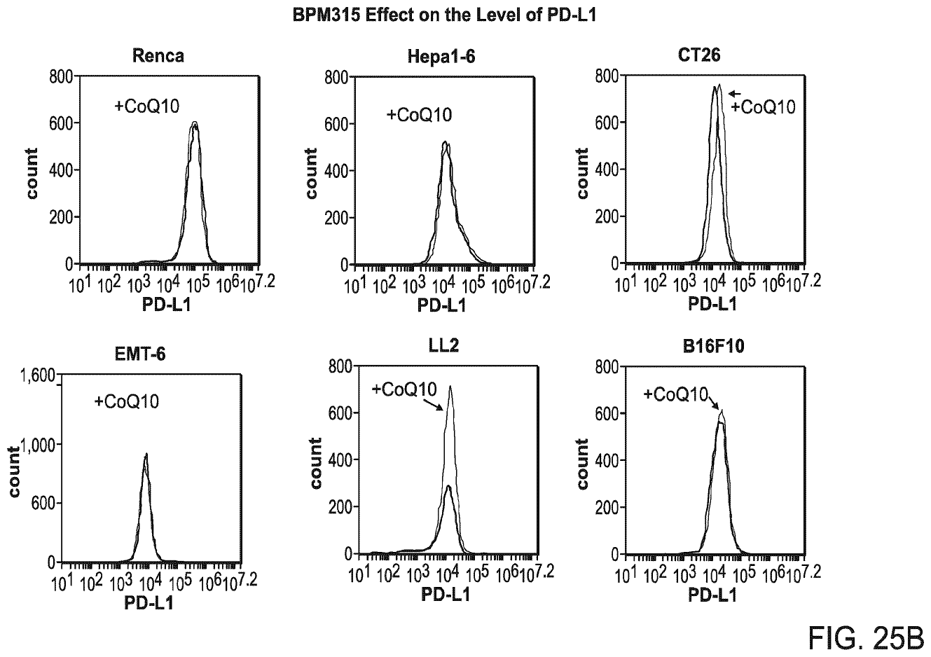

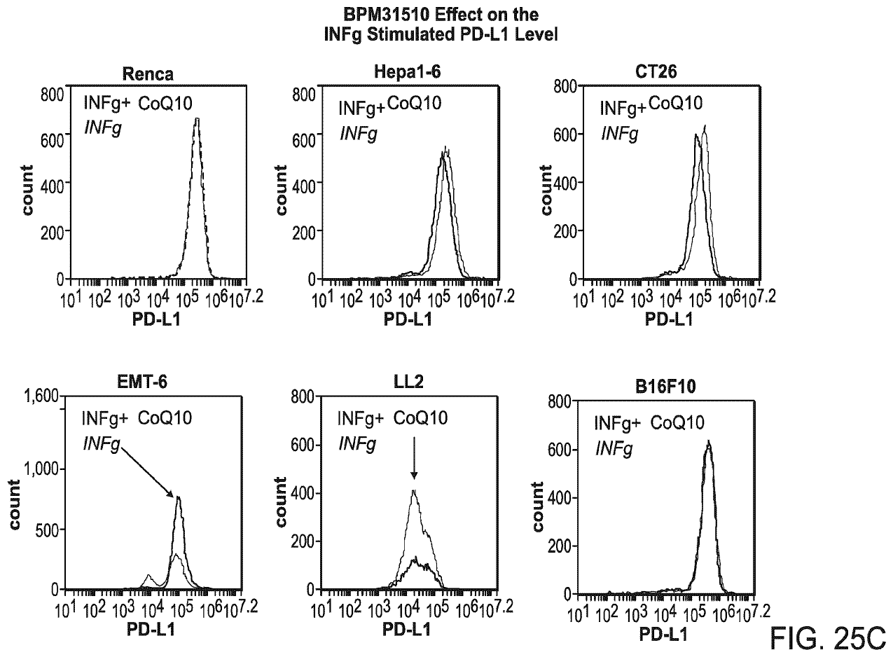

The effect of Coenzyme Q10 on the level of PD-L1 protein on the surface of the mouse syngeneic tumor cells was also evaluated. Mouse syngeneic tumor cell lines from different tissue types were cultured with or without interferon γ (INFγ) in the presence or absence of their corresponding IC₅₀ amount of Coenzyme Q10 at 37° C. for 24 hours. As shown in FIGS. 25A-25C, Coenzyme Q10 did not have any effect on the level of PD-L1 protein.

### Example 10—Effects of Coenzyme Q10 on Mouse Pancreatic Cancer In Vivo

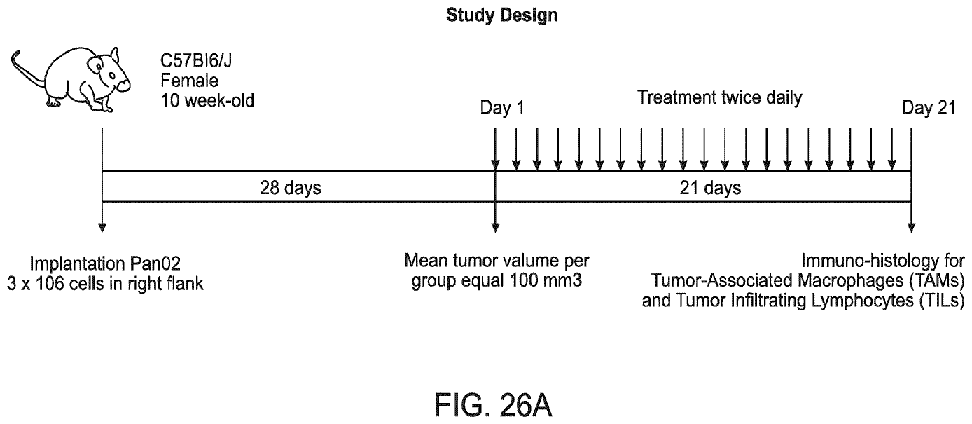

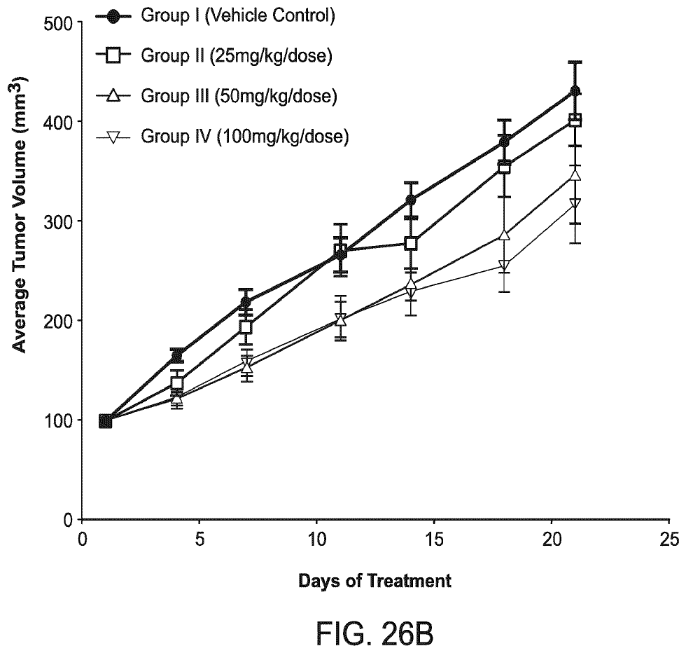

C57BL/6 mice were implanted with murine Pan02 pancreatic cancer cells and treated with different doses of Coenzyme Q10 to evaluate the effects of Coenzyme Q10 on tumor growth. C57Bl/6 female mice were inoculated with 3×10⁷ Pan02 cells. When tumors reached a mean volume of 100 mm³, animals were randomized into four groups and treated with vehicle control or Coenzyme Q10 (25, 50 or 100 mg/kg) by intraperitoneal (i.p.) injection twice daily for 21 days. Tumor volume was measured twice per week. An overview of the study design is shown in FIG. 26A, and the treatment groups are shown in Table 12 below.

TABLE 12
Treatment groups for murine Pan02 pancreatic
cancer study in C57BL/6 mice.

| Group | N | Mouse Strain | Agent | Route | Schedule |
|---|---|---|---|---|---|
| 1 | 10 | C57BL/6 | Saline | i.p. | Bid to end |
| | | | | | first day 1 |
| | | | | | dose |
| 2 | 10 | C57BL/6 | 25 mg/kg/dose | i.p. | Bid to end |
| | | | | | first day 1 |
| | | | | | dose |
| 3 | 10 | C57BL/6 | 50 mg/kg/dose | i.p. | Bid to end |
| | | | | | first day 1 |
| | | | | | dose |
| 4 | 10 | C57BL/6 | 100 mg/kg/dose | i.p. | Bid to end |
| | | | | | first day 1 |
| | | | | | dose |

The 25, 50 and 100 mg/kg doses of Coenzyme Q10 decreased tumor volume by 7%, 19% and 26% respectively by Day 21. See FIG. 26B and Table 13 below.

TABLE 13
Percentage of tumor growth inhibition of murine
Pan 02 pancreatic tumors in C57BL/6 mice.

| | Percentage of | Statistical Analysis |
|---|---|---|
| Groups | Tumor Growth Inhibition | (p-value to control) |
| Control Saline | | |
| 25 mg/kg | 7 | 0.2583 |
| 50 mg/kg | 19 | 0.1255 |
| 100 mg/kg | 26 | 0.0529 |

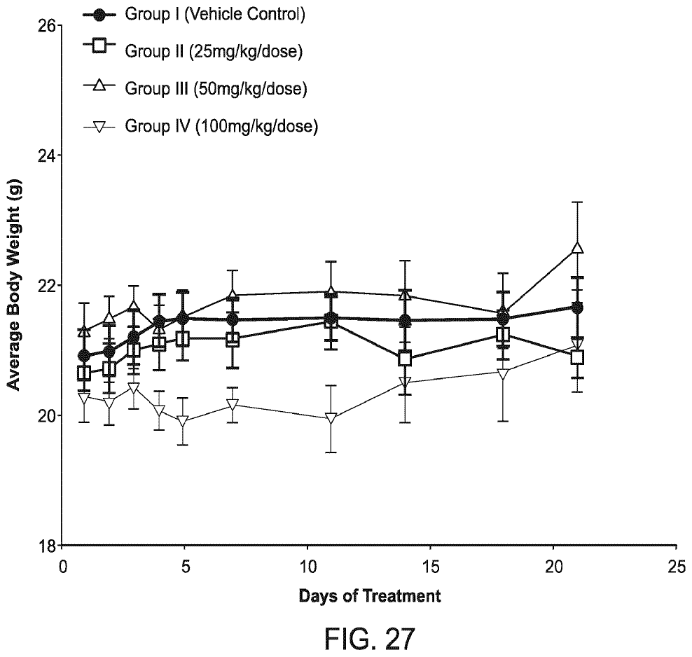

The body weight of C57BL/6 mice implanted with murine Pan02 pancreatic cancer cells and treated with Coenzyme Q10 was also evaluated. Tumors with mean volume of 100 mm³ were treated twice per day with vehicle control or Coenzyme Q10 at 25, 50 or 100 mg/kg administered intraperitoneally for 21 days. Body weight was measured every two days for the first 5 days, and then twice per week. As shown in FIG. 27, Coenzyme Q10 had no significant effect on the body weight of the animals.

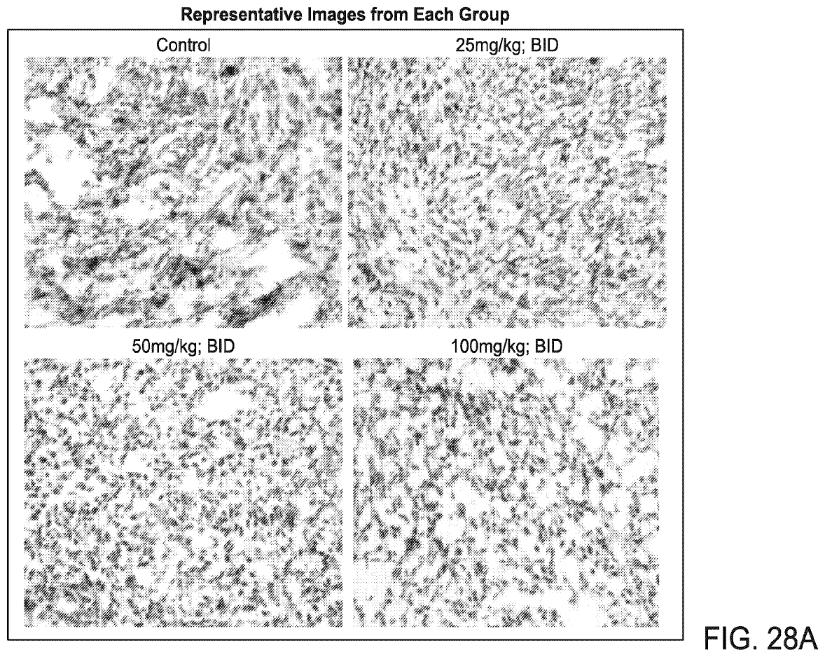

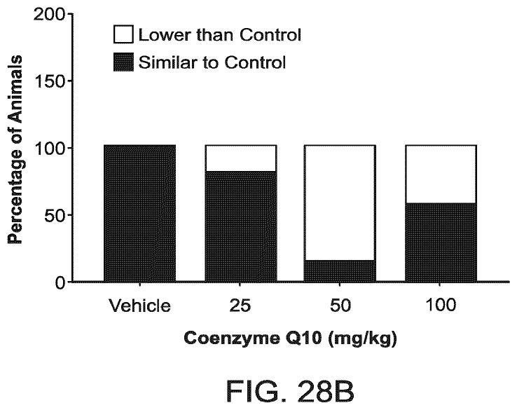

Pancreatic tumor samples from mice treated with different doses of Coenzyme Q10 were analyzed for the presence of tumor associated macrophages (TAMs). TAMs are found in close proximity to or within tumors and support tumor growth. C57Bl/6 female mice were inoculated with Pan02 cells. When tumors reached a mean volume of 100 mm³, animals were randomized into four groups and treated with vehicle control or Coenzyme Q10 (25, 50 or 100 mg/kg) twice daily for 21 days. At the end of the study, tumors were removed and subjected to immunohistochemistry (IHC) analysis for TAMs using the F4/80 marker. All slides were subjected to a pathological scoring. Scores were relative to a control slide (from the control group) which demonstrated the best level of intensity. As shown in FIGS. 28A and 28B, Coenzyme Q10 decreased TAMs in a dose dependent manner. Pathological scoring of TAMs in the tumors by F4/80 IHC is shown in Table 14 below.

TABLE 14
Pathological scoring of tumor associated macrophages
(TAMs) by F4/80 immunohistochemistry (IHC) analysis
in murine Pan02 pancreatic tumors in C57BL/6 mice.

| | Similar | Lower | Higher |
|---|---|---|---|
| | to Control | than Control | than Control |
| Group 1 (Vehicle) | 100% | | |
| Group II (25 mg/kg) | 80% | 20% | 0% |
| Group III (50 mg/kg) | 14.3% | 85.8% | 0% |
| Group IV (100 mg/kg) | 55.6% | 44.4% | 0% |

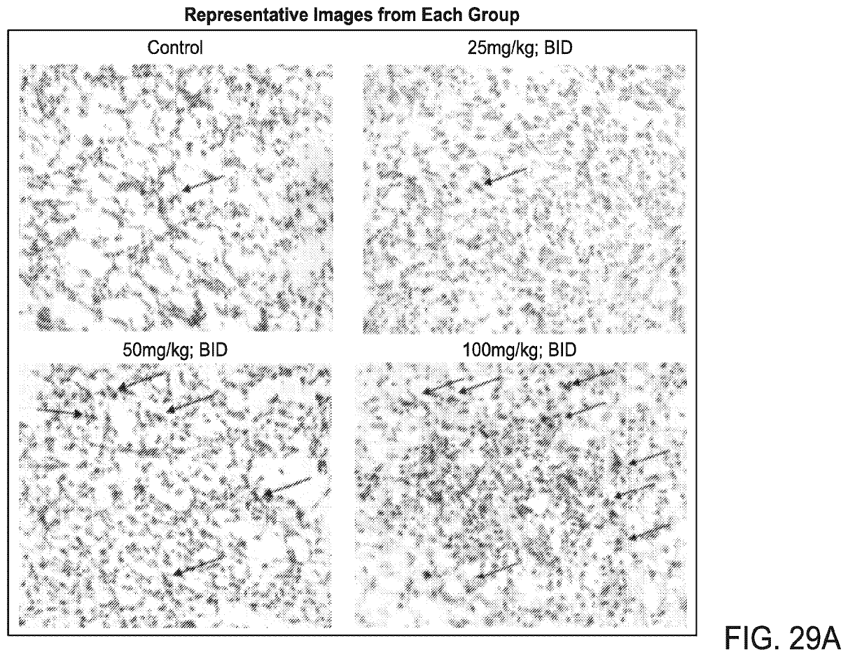

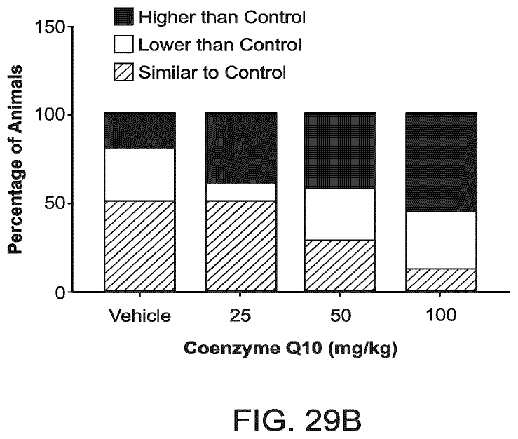

Tumor samples from mice with murine Pan02 tumors treated with different doses of Coenzyme Q10 were analyzed for the presence of tumor infiltrating lymphocytes (TILs). C57Bl/6 female mice were inoculated with 3×10⁷ Pan02 cells. When tumors reached a mean volume of 100 mm³, animals were randomized into four groups and treated with vehicle control or Coenzyme Q10 (25, 50 or 100 mg/kg) twice daily for 21 days. At the end of the study, tumors were removed and subjected to IHC analysis for Tumor Infiltrating Lymphocytes (TILs) with CD8 staining. All slides were subjected to a pathological scoring. Scores were relative to a control slide (from control group) which demonstrated the best level of intensity. As shown in FIGS. 29A and 29B, Coenzyme Q10 increased TILs in a dose-dependent manner. Pathological scoring of TILs in the tumors by CD8+ IHC is shown in Table 15 below.

TABLE 15
Pathological scoring of Tumor Infiltrating Lymphocytes
(TILs) by CD8+ immunohistochemistry (IHC) analysis
in murine Pan02 pancreatic tumors in C57BL/6 mice.

| | Similar | Lower | Higher |
|---|---|---|---|
| | to Control | than Control | than Control |
| Group 1 (Vehicle) | 50% | 30% | 20% |
| Group II (25 mg/kg) | 50% | 10% | 40% |
| Group III (50 mg/kg) | 28.6% | 28.6% | 42.9% |
| Group IV (100 mg/kg) | 11.1% | 33.3% | 55.2% |

In conclusion, Coenzyme Q10 selectively influenced activation and maturation of T cells in murine peripheral blood mononuclear cells (PBMCs). In addition, Coenzyme Q10 demonstrated a potent anti-tumor effect in a syngeneic pancreatic tumor model, Pan02. IHC analysis demonstrated that treatment with Coenzyme Q10 increased the level of TILs and decreases the level of TAMs. Accordingly, these data indicate that Coenzyme Q10 exerts potent anti-tumor effects through its dual function of modulating tumor cell metabolism and influencing immune checkpoint proteins to improve overall survival outcomes.

1. A method of treating an oncological disorder in a subject in need thereof, comprising:
(a) administering coenzyme Q10 (CoQ10) to the subject; and
(b) administering at least one immune checkpoint modulator of an immune checkpoint molecule to the subject;
such that the oncological disorder is treated.

2. The method of claim 1, wherein the immune checkpoint molecule is selected from the group consisting of CD27, CD28, CD40, CD122, OX40, GITR, ICOS, 4-1BB, ADORA2A, B7-H3, B7-H4, BTLA, CTLA-4, IDO, KIR, LAG-3, PD-1, PD-L1, PD-L2, TIM-3, and VISTA.

3. The method of claim 1 or 2, wherein the immune checkpoint molecule is selected from the group consisting of PD-1, PD-L1, PD-L2, CTLA-4, LAG-3, TIM-3 and VISTA.

4. The method of any one of claims 1-3, wherein the immune checkpoint molecule is selected from the group consisting of PD-1, PD-L1 and CTLA-4.

5. The method of claim 1 or 2, wherein the immune checkpoint molecule is a stimulatory immune checkpoint molecule.

6. The method of claim 5, wherein the immune checkpoint modulator is an agonist of the stimulatory immune checkpoint molecule.

7. The method of claim 1 or 2, wherein the immune checkpoint molecule is an inhibitory immune checkpoint molecule.

8. The method of claim 7, wherein the immune checkpoint modulator is an antagonist of the inhibitory immune checkpoint molecule.

9. The method of any one of claims 1-8, wherein the immune checkpoint modulator is selected from the group consisting of a small molecule, an inhibitory RNA, an antisense molecule, and an immune checkpoint binding protein.

10. The method of claim 9, wherein the immune checkpoint modulator is an immune checkpoint binding protein.

11. The method of claim 10, wherein the immune checkpoint binding protein is selected from the group consisting of an antibody, antibody Fab fragment, divalent antibody, antibody drug conjugate, scFv, fusion protein, bivalent antibody, and tetravalant antibody.

12. The method of claim 1 or 2, wherein the immune checkpoint modulator is PD-1.

13. The method of claim 12, wherein the immune checkpoint modulator is selected from the group consisting of pembrolizumab, novolumab, pidilizumab, SHR-1210, MEDI0680R01, BBg-A317, TSR-042, REGN2810 and PF-06801591.

14. The method of claim 1 or 2, wherein the immune checkpoint molecule is PD-L1.

15. The method of claim 14, wherein the immune checkpoint modulator is selected from the group consisting of durvalumab, atezolizumab, avelumab, MDX-1105, AMP-224 and LY3300054.

16. The method of claim 1 or 2, wherein the immune checkpoint molecule is CTLA-4.

17. The method of claim 16, wherein the immune checkpoint modulator is selected from the group consisting of ipilimumab, tremelimumab, JMW-3B3 and AGEN1884.

18. The method of claim 1 or 2, wherein the immune checkpoint molecule is LAG-3.

19. The method of claim 18, wherein the immune checkpoint modulator is selected from the group consisting of pembrolizumab, nivolumab, pidilizumab, SHR-1210, MEDI0680, PDR001, BGB-A317, TSR-042, REGN2810, and PF-06801591.

20. The method of claim 1 or 2, wherein the immune checkpoint molecule is TIM-3.

21. The method of claim 20, wherein the immune checkpoint modulator is selected from the group consisting of TSR-022 and MGB453.

22. The method of claim 1 or 2, wherein the immune checkpoint molecule is VISTA.

23. The method of claim 22, wherein the immune checkpoint modulator is selected from the group consisting of TSR-022 and MGB453.

24. The method of any one of claims 1 to 23, wherein the Coenzyme Q10 is administered before administration of the immune checkpoint modulator.

25. The method of any one of claims 1 to 23, wherein the Coenzyme Q10 is administered concurrently with the immune checkpoint modulator.

26. The method of any one of claims 1 to 23, wherein the Coenzyme Q10 is administered after administration of the immune checkpoint modulator.

27. The method of any one of claims 1 to 26, wherein a response of the oncological disorder to treatment is improved relative to a treatment with the at least one immune checkpoint modulator alone.

28. The method of claim 27, wherein the response in a population of patients is improved by at least 5%, at least 10%, at least 15%, at least 20%, at least 30%, at least 40%, at least 50%, at least 60%, at least 70%, at least 80% or more relative to treatment with the at least one immune checkpoint modulator alone.

29. The method of claim 27 or 28, wherein the response comprises any one or more of reduction in tumor burden, reduction in tumor size, inhibition of tumor growth, achieving stable oncological disorder in a subject with a progressive oncological disorder prior to treatment, increased time to progression of the oncological disorder, and increased time of survival.

30. The method of any one of claims 1-29, wherein the Coenzyme Q10 and the immune checkpoint modulator act synergistically.

31. The method of any one of claims 1 to 30, wherein the CoQ10 is administered topically.

32. The method of any one of claims 1 to 30, wherein the CoQ10 is administered by injection or infusion.

33. The method of claim 32, wherein the CoQ10 is administered by intravenous administration.

34. The method of claim 32, wherein the CoQ10 is administered by continuous intravenous infusion.

35. The method of claim 34, wherein the CoQ10 is administered by continuous infusion over between 24 and 96 hours.

36. The method of any one of claims 1 to 35, wherein the oncological disorder is selected from the group consisting of a carcinoma, sarcoma, lymphoma, melanoma, and leukemia.

37. The method of any one of claims 1 to 35, wherein the oncological disorder is selected from the group consisting of pancreatic cancer, breast cancer, liver cancer, skin cancer, lung cancer, colon cancer, prostate cancer, thyroid cancer, bladder cancer, rectal cancer, endometrial cancer, kidney cancer, bone cancer, brain cancer, cervical cancer, stomach cancer, mouth and oral cancers, neuroblastoma, testicular cancer, uterine cancer, and vulvar cancer.

38. The method of claim 37, wherein the skin cancer is selected from the group consisting of melanoma, squamous cell carcinoma, basal cell carcinoma, and cutaneous T-cell lymphoma (CTCL).

39. The method of any one of claims 1 to 38, wherein the subject is human.
